(19) United States
(12) Patent Application Publication
(10) Pub. No.: US 2024/0398783 A1
(43) Pub. Date: Dec. 5, 2024

(54) Compounds and Pharmaceutical Compositions Comprising Inhibitors of Amyloid Peptide Interactions with Glycosaminoglycans, Methods of Treatment and Use Thereof

(71) Applicant: Gismo Therapeutics, Inc., Lexington, KY (US)

(72) Inventors: Paul GREGOR, Howard Beach, NY (US); Myung Hee KIM, Lexington, KY (US); Brian MARRON, Ada, MI (US)

(21) Appl. No.: 18/688,859

(22) PCT Filed: Sep. 6, 2022
(86) PCT No.: PCT/US2022/042597

§ 371 Date: Mar. 4, 2024

Related U.S. Application Data
(60) Provisional application No. 63/241,148, filed on Sep. 7, 2021.

(51) Int. Cl. A61K 31/4709; A61K 31/4164; A61K 31/4706; A61K 31/4725; A61K 31/496; A61K 31/502; A61K 31/517; A61K 31/5377; A61K 31/541; C07D 215/42; C07D 233/64; C07D 239/94; C07D 401/12; C07D 401/14

(52) U.S. Cl. CPC A61K 31/4709; A61K 31/4164; A61K 31/4706; A61K 31/4725; A61K 31/496; A61K 31/502; A61K 31/517; A61K 31/5377; A61K 31/541; C07D 215/42; C07D 233/64; C07D 239/94; C07D 401/12; C07D 401/14

(57) ABSTRACT

The disclosure provides quinoline and quinazoline derivative compounds and pharmaceutical compositions thereof. These compounds inhibit the interactions between GAG-binding amyloid peptides (GBAPs) and heparan sulfate glycosaminoglycans (HS-GAGs) and thus may be useful as therapeutics for the treatment and prevention of neurodegenerative diseases associated with amyloidosis, for example Alzheimer's Disease, and for other amyloid disorders. The present disclosure further provides methods of treatment and prevention of neurodegenerative and amyloid diseases, and methods for identifying small organic molecule compounds that can inhibit the interaction of glycosaminoglycans (GAGs) with GAG-binding amyloid peptides (GBAPs).

This patent application claims the benefit of U.S. Prov. Ser. No. 63/241,148 filed Sep. 7, 2021, the entirety of which is incorporated herein by reference.

### FIELD OF THE INVENTION

The present disclosure relates in general to the field of discovering compounds for the treatment of amyloid disorders, including amyloid neurodegenerative diseases such as Alzheimer's Disease. Compounds that inhibit interactions of amyloid peptides with glycosaminoglycans (GAGs) are provided, as well as pharmaceutical compositions, and uses thereof.

### BACKGROUND OF THE INVENTION

Throughout the description of the disclosure reference is made to certain publications including scientific articles and patents or patent applications. It is the intent that each of these publications be incorporated by reference in their entirety when referred to in the specification.

Amyloid Disorders. Amyloid Disorders are associated with amyloidosis, the process in which amyloid peptides or proteins are misfolded and aggregated, abnormally deposited in organs and/or tissues (Chiti, F. and Dobson, C. M. (2017) Annual Review of Biochemistry, 86, 27-68). A protein or peptide is described as being amyloid if, due to an alteration in its secondary structure, it takes on a particular aggregated insoluble form (amyloid fibrils). There are about 30 different types of amyloidosis, each due to a specific protein misfolding. Symptoms vary widely depending upon the site of amyloid deposition. Amyloidosis may be inherited or acquired. Amyloid Disorders include, for instance, Alzheimer's Disease; Parkinson Disease; Amyotrophic Lateral Sclerosis, Prion Disease (also known as Variant Creutzfeld-Jacob Disease; Bovine Spongiform Encephalomyelitis; Mad Cow Disease); Amyloid Light Chain (AL) Amyloidosis and Secondary Amyloidosis.

Amyloid Neurodegenerative Disease. The misfolding and aggregation of specific proteins is an occurrence in a variety of neurodegenerative disorders. In Alzheimer disease, the two principal aggregating proteins are β-amyloid and tau. The abnormal assemblies formed by conformational variants of these proteins range in size from small oligomers to the characteristic lesions such as senile plaques and neurofibrillary tangles. Pathologic similarities with prion disease suggest that the formation and spread of these proteinaceous lesions might involve a common molecular mechanism-corruptive protein templating. (Jucker, M. and Walker, L. C. (2011) Ann Neurol 70(4):532-40).

Amyloid Peptides and Proteins. Amyloid diseases are caused, in part, by the self-association of an amyloid peptide or protein to form insoluble fibrillar complexes within and around cells thereby impeding normal cellular function. Some of the known amyloid peptides/proteins are Beta-amyloid (Abeta; β-amyloid) peptides including beta-amyloid (1-42) and beta-amyloid (1-40), prion protein, Tau, alpha-synuclein, TDP-43, islet amyloid polypeptide (IAPP), transthyretin, beta 2 microglobulin, serum amyloid A (SAA), and immunoglobulin light chain AL.

Beta-Amyloid (Abeta; β-amyloid; amyloid-beta). According to the “amyloid” hypothesis, the deposition of the Beta-Amyloid peptide in the brain is a central event in Alzheimer's Disease. The Alzheimer's brains contain neuritic plaques consisting of extracellular deposits of the amyloid peptide Beta-Amyloid. Amyloid plaque contains also other constituents, among them substantial amounts of glycosaminoglycans (GAGs). Beta-Amyloid has the propensity to undergo conformational change to an anti-parallel beta-sheet like structure and to aggregate (the aggregation of Beta-Amyloid may be induced also in vitro). This process is termed fibrillogenesis and it results in the formation of amyloid fibrils. Beta-Amyloid can be toxic to cultured mammalian cells in vitro, especially in the form of Abeta oligomers (Levine, H., 3rd (2007) Amyloid 14(3):185-97).

Alpha-Synuclein (α-Synuclein) is a neuronal protein that is linked genetically and neuropathologically to Parkinson's disease. It is generally thought that α-synuclein aberrant soluble oligomeric conformations, termed protofibrils, are the toxic species that mediate disruption of cellular homeostasis and neuronal death, through effects on various intracellular targets, including synaptic function (Stefanis, L. (2012), Cold Spring Harb Perspect Med. 2(2)). Furthermore, secreted α-synuclein may exert deleterious effects on neighboring cells, including seeding of aggregation, thus possibly contributing to disease propagation. Targeting the toxic functions conferred by this protein may lead to novel therapeutic strategies for Parkinson's Disease and synucleinopathies.

Tau. The tau proteins (r proteins)) are a group of soluble proteins produced by alternative splicing from the gene MAPT (microtubule-associated protein tau). They have roles primarily in maintaining the stability of microtubules in axons and are abundant in the neurons of the central nervous system. Pathologies and dementias of the nervous system such as Alzheimer's disease and Parkinson's disease are associated with tau proteins that have become hyperphosphorylated insoluble aggregates called neurofibrillary tangles. The term “prion-like” is often used to describe several aspects of tau pathology in various tauopathies, like Alzheimer's disease and frontotemporal dementia. Prions are defined by their ability to induce misfolding of native proteins to perpetuate the pathology. Pathological tau aggregates have been shown to have the capacity to induce misfolding of native tau protein (Congdon, E. E. and Sigurdsson, E. M. Nat Rev Neurol. 2018 July; 14(7): 399-415).

TAR DNA-binding protein 43 (TDP-43). TDP-43 proteinopathies (Jucker and Walker, ibid), consisting of several neurodegenerative diseases, including frontotemporal lobar dementia (FTLD) and amyotrophic lateral sclerosis (ALS), are characterized by inclusion bodies formed by polyubiquitinated and hyperphosphorylated full-length and truncated TDP-43. TDP-43 may form structurally stable, spherical oligomers that are neurotoxic in vitro and in vivo. Such oligomers are present in the forebrain of transgenic TDP-43 mice and FTLD-TDP patients.

Serum Amyloid A (SAA). SAA are peptides which during the acute phase response may rise as much as 1000-fold in serum. Fragments of SAA can form highly organized, insoluble fibrils that accumulate in “secondary” amyloid disease (Sack, G. H. (2018) Serum amyloid A—a review. Mol Med 24, 46).

Glycosaminoglycans (also referred to herein and in the art as “GAG” or “GAGs”) are naturally-occurring carbohydrate-based molecules implicated in the regulation of a number of cellular processes, most likely by interaction with effector molecules (Lindahl, U. and Kjellen, L. (2013) J Intern Med 273(6):555-71). GAGs are linear, non-branched chains of repeating two-sugar (disaccharide) units, which may be up to 150 units in length. In vivo, GAGs are typically linked to specific proteins thus forming proteoglycans. All GAGs (with the exception of hyaluronic acid) contain sulfate groups variously esterified to the ring hydroxyl groups of the sugars. These negatively charged groups are believed to figure prominently in the biological properties attributed to GAGs. There are four main types of sulfated GAGs: (1) Heparan Sulfate (HS-GAG); (2) Chondroitin Sulfate (CS-GAG); (3) Keratan Sulfate (KS-GAG); and (4) Dermatan Sulfate (DS-GAG). HS-GAG is structurally very similar to heparin. Its complex biosynthesis results in a variably sulfated disaccharide repeat. HS-GAGs contain binding sites for many biologically active peptides and proteins; the interaction is mediated by heparin-binding domains. Due to highly diverse sulfation pattern it is estimated that HS-GAGs could contain up to a million diverse possible binding sites. In tissues, GAGs are linked to membrane proteins forming proteoglycans. Heparan Sulfate Proteoglycans (HSPGs) are ubiquitous macromolecules associated with the cell surface and the extracellular matrix of a wide range of cells of vertebrate and invertebrate tissues. The basic HSPG structure consists of a protein core to which several linear heparan sulfate chains are covalently attached (Lindahl and Kjellen, 2013, ibid). Three major families of proteoglycan core proteins have been characterized: the membrane-spanning syndecans, the glycosylphosphatidylinositol-linked glypicans, and the basement membrane PGs perlecan and agrin.

Heparin, a widely used anticoagulant, is one of the most thoroughly studied GAGs. It is a highly sulfated form of heparan sulfate found mainly in mast cells. As a commercial product, heparin is a hetero-oligodisaccharide composition of about 20-60 monomeric units. It has no protein associated with it. Heparin is commonly used in biochemical and binding assays instead of HS-GAGs, because of their similarity (Lindahl, U. and Kjellen, L. (2013) ibid).

Heparin-binding domains. Many biologically active peptides and proteins have heparin-binding domains that bind to GAGs (Lindahl and Kjellen, 2013, ibid). These include chemokines, cytokines, growth factors, viral envelope proteins, amyloid peptides, fibronectin, etc. Generally, there are no amino acid sequence homologies between different heparin-binding domains, thus providing the molecular basis for selectivity. Although interactions of proteins with GAGs, such as heparin and heparan sulphate, are of great biological importance, structural requirements for protein-GAG binding have not been well-characterized. Ionic interactions are important in promoting protein-GAG binding. Despite their identical charges, arginine residues bind more tightly to GAGs than lysine residues. Most amyloid peptides and proteins, including beta-amyloid, alpha-synuclein, TDP-43, SAA and Tau, have heparin binding domains.

Glycosaminoglycans (GAGs) and Amyloid Disorders. GAGs such as HS-GAGs have been implicated in the etiology of amyloid diseases (Maiza A. et al. (2018) FEBS Lett 592(23):3806-3818). HS-GAGs may promote fibrillogenesis by associating with the amyloid precursors and inducing the conformational change required for their assembly into fibrils. HS-GAGs also remain associated with the nascent fibrils contributing to their stability. The heparin binding region of Beta-amyloid has been defined as a positively charged domain HisGlnLysLys corresponding to position #13-16 in the Beta-amyloid sequence. It has been shown that Beta-amyloid interacts with high affinity with HS-GAGs in vitro via direct binding. HS-GAGs as well as other sulfated GAGs and heparin accelerate Beta-amyloid beta-sheet conformation, accompanied by fibril formation. Sulfated GAGs and HS-GAGs have been implicated in other amyloid diseases associated with amyloidogenic proteins such as Tau or alpha-synuclein (Maiza, A., ibid).

HS-GAGs are implicated in spreading prion-like proteopathic seeds across the nervous system. Recent experimental evidence suggests that transcellular propagation of fibrillar protein aggregates drives the progression of neurodegenerative diseases in a prion-like manner (Holmes, B. B. et al. (2013) Proc Natl Acad Sci USA, 110(33): E3138-E3147). This phenomenon is now well described in cell and animal models and involves the release of protein aggregates into the extracellular space. Free aggregates then enter neighboring cells to seed further fibrillization. Prion-like propagation of proteopathic seeds may underlie the progression of neurodegenerative diseases, including the tauopathies and synucleinopathies. Aggregate entry into the cell is a crucial step in transcellular propagation. Heparan sulfate proteoglycans have been shown as critical mediators of tau aggregate binding and uptake, and subsequent seeding of normal intracellular tau. This pathway mediates aggregate uptake in cultured cells, primary neurons, and brain. α-Synuclein fibrils use the same entry mechanism to seed intracellular aggregation. This establishes the molecular basis for a key step in aggregate propagation.

Discovery and development of therapeutics for amyloid diseases. Amyloid disorders are associated with amyloidogenic polypeptides. Thus, drugs which are able to selectively block the aggregation, misfolding, propagation or deposition of amyloid proteins may be expected to provide prophylactic and/or therapeutic benefit in a variety of amyloidoses, for example, Alzheimers Disease, inflammatory amyloidosis, and prion disease. In spite of considerable work, efforts to develop effective therapeutics for amyloid diseases such as Alzheimer's Disease have had only very limited success and there is a need to develop new therapeutics.

GAG mimetics. One method to treat AD that was previously disclosed involves GAG mimetics. For instance, certain small molecule sulfated compounds inhibit binding of amyloid peptides to GAGs and display in vivo activity in animal models of AD (Kisilevsky, R., 1996, Drugs Aging 8, 75-83); however, such sulfated compounds do not bind GAGs but rather interact directly with amyloid peptides.

One objective of the present disclosure is discovery of small molecule compounds capable of inhibiting interactions between GAGs and amyloid peptides. As described herein, it is anticipated that such compounds may be useful in prevention and treatment of amyloid disorders.

Another objective is to identify new drug targets for amyloid disorders associated with amyloid peptides.

Another objective is to develop simple reproducible GAG-amyloid peptide binding assays for screening compound libraries, such as by HTS, using 96-well or other multi-well plates and standard detection methods such as spectroscopy.

The following patent application describes Quinolin-4-Yl hydrazine Derivatives As Antimalarial Agents. Campiani, G. et al WO 2007/104695.

Each of the references cited herein is incorporated by reference in its entirety. The publications discussed herein are provided solely for their disclosure. Nothing herein is to be construed as an admission that the present disclosure is not entitled to antedate such publication by virtue of prior invention. Actual publication dates may need to be confirmed independently.

### BRIEF SUMMARY OF THE INVENTION

The present disclosure provides small molecule compounds that inhibit binding of GAG-binding amyloid peptides (GBAPs) to heparan sulfate glycosaminoglycans (HS-GAGs) and thus may be useful as therapeutics, for instance for Alzheimer's Disease as well as other amyloid and neurodegenerative diseases. Also described are pharmaceutical compositions, uses thereof, and methods of screening.

According to one embodiment the present disclosure provides compound of a general formula I:

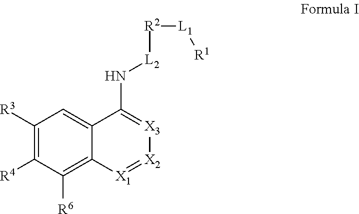

and a pharmaceutically acceptable diluent or carrier
   wherein
X₁, X₂, and X₃ are independent and optionally —CR⁹— or N; R⁹ is alkyl, halogen, —O-alkyl. If X₁ is CR⁹ then R⁹ and R⁶ can be joined to form a 5-7 member carbocyclic, heteroaromatic or heterocyclic ring;
R³ and R⁴ are independent and optionally H, halogen, —O-alkyl. R³ and R⁴ can be joined to form a fused phenyl;
R⁵ is H, C1-6 alkyl;
R² is phenyl optionally substituted with C1-6 alkyl, —OH, —O-alkyl, halogen, heterocycle, heteroaromatic; 5 or 6 member heterocycle or heteroaromatic optionally substituted with C1-6 alkyl, —OH, —O-alkyl, halogen; a 5 or 6-member heterocycle optionally substituted with alkyl, —O-alkyl, halogen, heterocycle, heteroaromatic; —N+(CH3)3, optionally substituted 4, 5 and 6-member heterocycle including heterocycles and heteroaromatics that may contain a quaternized nitrogen to form a charged species:
L₁ is a bond, —CH3, —CH2-, —[CR7R8]n- where R⁷ and R⁸ are independently: F, C₁₋₆ alkyl, joined to form a C3-6 carbocycle or heterocycle or heteroaromatic.
n=0, 1, 2, 3;
L₂ can be a bond or —N═C(R5)-;
R₁ is null, a 5 or 6-member heterocycle optionally substituted with alkyl, —O-alkyl, halogen; —N+(CH3)3, optionally substituted 4, 5 and 6-member heterocycle including heterocycles and heteroaromatics that may contain a quaternized nitrogen to form a charged species;
R₆ is alkyl;
and pharmaceutically acceptable salts thereof. Also included are all the geometrical isomers about the carbon-nitrogen double bonds and possible tautomers.

Chemical synthesis of such compounds is described in Examples 1-42. Chemical structures are shown in Table 1. Analytical chemistry data of the compounds such as mass spectrometry and NMR data analysis are described in Examples.

As shown in Examples 44-48 and 50-54, the compounds of Formula I inhibit the interaction between GBAPs and HS-GAGs, and thus may be useful as therapeutics of neurodegenerative diseases associated with amyloidosis, and other amyloid diseases.

According to one embodiment, the compound of formula I that inhibits binding of a GBAP to HS-GAGs and is selected from the group consisting of:
(E)-4-((E)-(4-(1H-imidazol-1-yl)-2-methylbenzylidene)hydrazono)-7-chloro-1,4-dihydroquinoline (Compound 1)
(E)-7-chloro-4-((E)-(4-(4,5-dimethyl-1H-imidazol-1-yl)benzylidene)hydrazono)-1,4-dihydroquinoline (Compound 2)
(E)-4-((E)-(4-(1H-pyrazol-1-yl)benzylidene)hydrazono)-7-chloro-1,4-dihydroquinoline (Compound 3)
(Z)-1-(4-((2-(6-chloroquinolin-4-yl)hydrazineylidene)methyl)phenyl)—N,N,N-trimethylmethanaminium iodide (Compound 4)
(E)-4-(2-(4-(1H-imidazol-1-yl)benzylidene)hydrazinyl)-7-fluoroquinoline (Compound 5)
(E)-4-(4-((2-(7-fluoroquinolin-4-yl)hydrazono)methyl)benzyl)morpholine (Compound 6)
(E)-7-chloro-4-(2-(4-((4-methylpiperazin-1-yl)methyl)benzylidene)hydrazinyl)quinolone (Compound 7)
(E)-1-(4-((2-(1,2-dihydroacenaphthylen-5-yl)hydrazineylidene)methyl)phenyl)-1H-imidazole (Compound 8)
(Z)-1-methyl-4-((2-(quinolin-4-yl)hydrazono)methyl)pyridin-1-ium iodide (Compound 9)
(E)-4-(4-((2-(7-fluoroquinolin-4-yl)hydrazono)methyl)benzyl)thiomorpholine 1,1-dioxide (Compound 10)
4-(2-(4-(1H-imidazol-1-yl)-3-methylbenzylidene)hydrazineyl)-6,7-dimethoxyquinoline hydrochloride (Compound 11)
4-(2-(4-(1H-imidazol-1-yl)-3-methylbenzylidene)hydrazineyl)-7-methoxyquinoline (Compound 12)
(E)-4-(2-(4-(1H-imidazol-1-yl)-3-methoxybenzylidene)hydrazineyl)-6,7-dimethoxyquinoline phosphate (Compound 13)
(E)-4-(2-((6-(1H-imidazol-1-yl)pyridin-3-yl)methylene)hydrazineyl)-7-chloroquinoline hydrochloride (Compound 14)
7-chloro-4-(2-((6-(4,5-dimethyl-1H-imidazol-1-yl)pyridin-3-yl)methylene)hydrazineyl)quinazoline hydrochloride (Compound 15)
7-chloro-4-(2-(1-(6-(4,5-dimethyl-1H-imidazol-1-yl)pyridin-3-yl)ethylidene)hydrazineyl)quinazoline phosphate (Compound 16)
(E)-1-(5-(1-(2-(7-chloroquinazolin-4-yl)hydrazineylidene)ethyl)pyridin-2-yl)-3-methyl-1H-imidazol-3-ium iodide phosphoric acid salt (Compound 17)
(E)-7-chloro-4-(2-(1-(6-(2,4-dimethyl-1H-imidazol-1-yl)pyridin-3-yl)ethylidene)hydrazineyl)quinazoline phosphoric acid salt (Compound 18)
(E)-4-(2-(1-(6-(2,4-dimethyl-1H-imidazol-1-yl)pyridin-3-yl)ethylidene)hydrazineyl)quinazoline (Compound 19)
7-chloro-N-(2-(4-methylpiperazin-1-yl)pyridin-4-yl)quinolin-4-amine (Compound 20)
(E)-4-(2-(1-(6-(4,5-dimethyl-1H-imidazol-1-yl)pyridin-3-yl)ethylidene)hydrazineyl)-7-fluoroquinazoline (Compound 21)
7-chloro-4-(2-(1-(6-(4,5-dimethyl-1H-imidazol-1-yl)pyridin-3-yl)ethylidene)hydrazineyl)quinoline (Compound 22)
1-(5-(1-(2-(7-chloroquinolin-4-yl)hydrazineylidene)ethyl)pyridin-2-yl)-3-methyl-1H-imidazol-3-ium iodide (Compound 23)
(E)-6-chloro-1-(2-(1-(6-(2,4-dimethyl-1H-imidazol-1-yl)pyridin-3-yl)ethylidene)hydrazineyl)phthalazine (Compound 24)
7-chloro-N-(6-(4,5-dimethyl-1H-imidazol-1-yl)pyridin-3-yl)quinazolin-4-amine (Compound 25)
(E)-1-(5-(1-(2-(7-fluoroquinazolin-4-yl)hydrazineylidene)ethyl)pyridin-2-yl)-3-methyl-1H-imidazol-3-ium phosphoric acid salt (Compound 26)
(E)-7-chloro-4-(2-(1-(6-(2,4-dimethyl-1H-imidazol-1-yl)pyridin-3-yl)ethylidene)hydrazineyl)quinazoline hydrochloride (Compound 27)
7-chloro-4-(2-(1-(2-(4,5-dimethyl-1H-imidazol-1-yl)pyridin-4-yl)ethylidene)hydrazineyl)quinazoline (Compound 28)
1-(3-(1-(2-(7-chloroquinazolin-4-yl)hydrazineylidene)ethyl)phenyl)—N,N-dimethylmethanamine (Compound 29)
7-chloro-4-(2-(1-(3-(4-methylpiperazin-1-yl)phenyl)ethylidene)hydrazineyl)quinazoline (Compound 30)
6-chloro-N-(6-(4,5-dimethyl-1H-imidazol-1-yl)-5-methoxypyridin-3-yl)phthalazin-1-amine phosphoric acid salt (Compound 31)
(E)-4-(2-(1-(6-(1H-1,2,4-triazol-1-yl)pyridin-3-yl)ethylidene)hydrazineyl)-7-chloroquinazoline (Compound 32)
7-chloro-N-(6-(4,5-dimethyl-1H-imidazol-1-yl)-5-methoxypyridin-3-yl)isoquinolin-4-amine phosphoric acid salt (Compound 33)
N-(6-(1H-1,2,4-triazol-1-yl)pyridin-3-yl)-7-chloroquinazolin-4-amine phosphoric acid salt (Compound 34)
(E)-7-chloro-4-(2-(3-fluoro-4-(1H-imidazol-1-yl)benzylidene)hydrazineyl)quinoline hydrochloride (Compound 35)
7-chloro-N-(6-(2,4-dimethyl-1H-imidazol-1-yl)pyridin-3-yl)isoquinolin-4-amine phosphoric acid salt (Compound 37)
5-((7-chloroquinazolin-4-yl)amino)-2-(4,5-dimethyl-1H-imidazol-1-yl)pyridin-3-ol phosphoric acid salt (Compound 40)
N-(6-(1H-imidazol-1-yl)pyridin-3-yl)-7-chloroquinazolin-4-amine (Compound 41)
7-chloro-N-(3-((diethylamino)methyl)phenyl)quinolin-4-amine (Compound 42)
1-(5-((7-chloroquinazolin-4-yl)amino)pyridin-2-yl)-3-methyl-1H-imidazol-3-ium iodide phosphoric acid salt (Compound 43)
N-(6-(1H-1,2,4-triazol-1-yl)pyridin-3-yl)-7-chloroquinolin-4-amine (Compound 44) and
7-chloro-N-(2-((dimethylamino)methyl)pyridin-4-yl)quinolin-4-amine (Compound 45).

These compounds have biological activities as described in Examples, and thus may be useful as therapeutics for neurodegenerative and other amyloid diseases.

According to one embodiment the disclosure provides a pharmaceutical composition comprising a compound of general formula I:

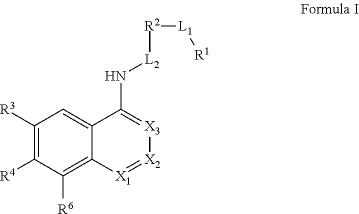

and a pharmaceutically acceptable diluent or carrier
wherein
X₁, X₂, and X₃ are independent and optionally —CR⁹— or N; R⁹ is alkyl, halogen, —O-alkyl. If X₁ is CR⁹ then R⁹ and R⁶ can be joined to form a 5-7 member carbocyclic, heteroaromatic or heterocyclic ring;
R³ and R⁴ are independent and optionally H, halogen, —O-alkyl. R³ and R⁴ can be joined to form a fused phenyl;
R⁵ is H, C1-6 alkyl;
R² is phenyl optionally substituted with C1-6 alkyl, —OH, —O-alkyl, halogen, heterocycle, heteroaromatic; 5 or 6 member heterocycle or heteroaromatic optionally substituted with C1-6 alkyl, —OH, —O-alkyl, halogen; a 5 or 6-member heterocycle optionally substituted with alkyl, —O-alkyl, halogen, heterocycle, heteroaromatic; —N+(CH3)3, optionally substituted 4, 5 and 6-member heterocycle including heterocycles and heteroaromatics that may contain a quaternized nitrogen to form a charged species:
L₁ is a bond, —CH3, —CH2-, —[CR7R8]n- where R7 and R8 are independently: F, C1-6 alkyl, joined to form a C3-6 carbocycle or heterocycle or heteroaromatic.
n=0, 1, 2, 3;
L₂ can be a bond or —N═C(R5)-;
R₁ is null, a 5 or 6-member heterocycle optionally substituted with alkyl, —O-alkyl, halogen; —N+(CH3)3, optionally substituted 4, 5 and 6-member heterocycle including heterocycles and heteroaromatics that may contain a quaternized nitrogen to form a charged species;
R₆ is alkyl;
and pharmaceutically acceptable salts thereof, the pharmaceutical composition being characterized by its ability to inhibit binding of a GAG-binding amyloid peptide (GBAP) to a HS-GAGs.

As described in Examples, such pharmaceutical compositions contain compounds that inhibit interactions between GBAPs and HS-GAGs and therefore, such pharmaceutical compositions may be useful for prevention or treatment of amyloid and neurodegenerative diseases.

According to another embodiment, a method of treating an amyloid disease or disorder is provided, said method comprising the steps of: selecting a subject in need of the treating and/or preventing an amyloid disease or disorder, administering to a subject in need thereof a therapeutically effective amount of a compound according to formula I, which compound is inhibiting GBAP binding to the HS-GAG, thereby treating and/or preventing the amyloid disease or disorder. According to one embodiment, GBAP is beta-amyloid and the amyloid disorder is Alzheimer's Disease or Cerebral Amyloid Angiopathy. According to another embodiment, GBAP is alpha-synuclein and the amyloid disorder is Parkinson's Disease or Multiple System Atrophy (MSA) or a synucleinopathy. According to another embodiment, GBAP is Tau and the amyloid disorder is Alzheimer's Disease, Frontotemporal Dementia or a tauopathy. According to another embodiment, GBAP is TDP-43 and the amyloid disorder is ALS or dementia associated with TDP-43 amyloidosis. According to another embodiment, GBAP is SAA and the amyloid disease or disorder is associated with Amyloid A (AA) amyloidosis.

According to one embodiment the pharmaceutical composition comprising a compound of general formula I is selected from the group consisting of
(E)-4-((E)-(4-(1H-imidazol-1-yl)-2-methylbenzylidene)hydrazono)-7-chloro-1,4-dihydroquinoline (Compound 1)
(E)-7-chloro-4-((E)-(4-(4,5-dimethyl-1H-imidazol-1-yl)benzylidene)hydrazono)-1,4-dihydroquinoline (Compound 2)
(E)-4-((E)-(4-(1H-pyrazol-1-yl)benzylidene)hydrazono)-7-chloro-1,4-dihydroquinoline (Compound 3)
(Z)-1-(4-((2-(6-chloroquinolin-4-yl)hydrazineylidene)methyl)phenyl)—N,N,N-trimethylmethanaminium iodide (Compound 4)
(E)-4-(2-(4-(1H-imidazol-1-yl)benzylidene)hydrazinyl)-7-fluoroquinoline (Compound 5)
(E)-4-(4-((2-(7-fluoroquinolin-4-yl)hydrazono)methyl)benzyl)morpholine (Compound 6)
(E)-7-chloro-4-(2-(4-((4-methylpiperazin-1-yl)methyl)benzylidene)hydrazinyl)quinolone (Compound 7)
(E)-1-(4-((2-(1,2-dihydroacenaphthylen-5-yl)hydrazineylidene)methyl)phenyl)-1H-imidazole (Compound 8)
(Z)-1-methyl-4-((2-(quinolin-4-yl)hydrazono)methyl)pyridin-1-ium iodide (Compound 9)
(E)-4-(4-((2-(7-fluoroquinolin-4-yl)hydrazono)methyl)benzyl)thiomorpholine 1,1-dioxide (Compound 10)
4-(2-(4-(1H-imidazol-1-yl)-3-methylbenzylidene)hydrazineyl)-6,7-dimethoxyquinoline hydrochloride (Compound 11)
4-(2-(4-(1H-imidazol-1-yl)-3-methylbenzylidene)hydrazineyl)-7-methoxyquinoline (Compound 12)
(E)-4-(2-(4-(1H-imidazol-1-yl)-3-methoxybenzylidene)hydrazineyl)-6,7-dimethoxyquinoline phosphate (Compound 13)
(E)-4-(2-((6-(1H-imidazol-1-yl)pyridin-3-yl)methylene)hydrazineyl)-7-chloroquinoline hydrochloride (Compound 14)
7-chloro-4-(2-((6-(4,5-dimethyl-1H-imidazol-1-yl)pyridin-3-yl)methylene)hydrazineyl)quinazoline hydrochloride (Compound 15)
7-chloro-4-(2-(1-(6-(4,5-dimethyl-1H-imidazol-1-yl)pyridin-3-yl)ethylidene)hydrazineyl)quinazoline phosphate (Compound 16)
(E)-1-(5-(1-(2-(7-chloroquinazolin-4-yl)hydrazineylidene)ethyl)pyridin-2-yl)-3-methyl-1H-imidazol-3-ium iodide phosphoric acid salt (Compound 17)
(E)-7-chloro-4-(2-(1-(6-(2,4-dimethyl-1H-imidazol-1-yl)pyridin-3-yl)ethylidene)hydrazineyl)quinazoline phosphoric acid salt (Compound 18)
(E)-4-(2-(1-(6-(2,4-dimethyl-1H-imidazol-1-yl)pyridin-3-yl)ethylidene)hydrazineyl)quinazoline (Compound 19)
7-chloro-N-(2-(4-methylpiperazin-1-yl)pyridin-4-yl)quinolin-4-amine (Compound 20)
(E)-4-(2-(1-(6-(4,5-dimethyl-1H-imidazol-1-yl)pyridin-3-yl)ethylidene)hydrazineyl)-7-fluoroquinazoline (Compound 21)
7-chloro-4-(2-(1-(6-(4,5-dimethyl-1H-imidazol-1-yl)pyridin-3-yl)ethylidene)hydrazineyl)quinoline (Compound 22)
1-(5-(1-(2-(7-chloroquinolin-4-yl)hydrazineylidene)ethyl)pyridin-2-yl)-3-methyl-1H-imidazol-3-ium iodide (Compound 23)
(E)-6-chloro-1-(2-(1-(6-(2,4-dimethyl-1H-imidazol-1-yl)pyridin-3-yl)ethylidene)hydrazineyl)phthalazine (Compound 24)
7-chloro-N-(6-(4,5-dimethyl-1H-imidazol-1-yl)pyridin-3-yl)quinazolin-4-amine (Compound 25)
(E)-1-(5-(1-(2-(7-fluoroquinazolin-4-yl)hydrazineylidene)ethyl)pyridin-2-yl)-3-methyl-1H-imidazol-3-ium phosphoric acid salt (Compound 26)
(E)-7-chloro-4-(2-(1-(6-(2,4-dimethyl-1H-imidazol-1-yl)pyridin-3-yl)ethylidene)hydrazineyl)quinazoline hydrochloride (Compound 27)
7-chloro-4-(2-(1-(2-(4,5-dimethyl-1H-imidazol-1-yl)pyridin-4-yl)ethylidene)hydrazineyl)quinazoline (Compound 28)
1-(3-(1-(2-(7-chloroquinazolin-4-yl)hydrazineylidene)ethyl)phenyl)—N,N-dimethylmethanamine (Compound 29)
7-chloro-4-(2-(1-(3-(4-methylpiperazin-1-yl)phenyl)ethylidene)hydrazineyl)quinazoline (Compound 30)
6-chloro-N-(6-(4,5-dimethyl-1H-imidazol-1-yl)-5-methoxypyridin-3-yl)phthalazin-1-amine phosphoric acid salt (Compound 31)
(E)-4-(2-(1-(6-(1H-1,2,4-triazol-1-yl)pyridin-3-yl)ethylidene)hydrazineyl)-7-chloroquinazoline (Compound 32)
7-chloro-N-(6-(4,5-dimethyl-1H-imidazol-1-yl)-5-methoxypyridin-3-yl)isoquinolin-4-amine phosphoric acid salt (Compound 33)
N-(6-(1H-1,2,4-triazol-1-yl)pyridin-3-yl)-7-chloroquinazolin-4-amine phosphoric acid salt (Compound 34)
(E)-7-chloro-4-(2-(3-fluoro-4-(1H-imidazol-1-yl)benzylidene)hydrazineyl)quinoline hydrochloride (Compound 35)
4-(2-(1-(4-(1H-imidazol-1-yl)phenyl)ethylidene)hydrazineyl)-7-chloroquinoline phosphate (Compound 36)
7-chloro-N-(6-(2,4-dimethyl-1H-imidazol-1-yl)pyridin-3-yl)isoquinolin-4-amine phosphoric acid salt (Compound 37)
4-(2-(4-(1H-imidazol-1-yl)benzylidene)hydrazineyl)-7-chloroquinoline dihydrochloride (Compound 38)
N-(4-(4-ethylpiperazin-1-yl)phenyl)benzo[g]quinolin-4-amine (Compound 39) 5-((7-chloroquinazolin-4-yl)amino)-2-(4,5-dimethyl-1H-imidazol-1-yl)pyridin-3-ol phosphoric acid salt (Compound 40)
N-(6-(1H-imidazol-1-yl)pyridin-3-yl)-7-chloroquinazolin-4-amine (Compound 41)
7-chloro-N-(3-((diethylamino)methyl)phenyl)quinolin-4-amine (Compound 42)
1-(5-((7-chloroquinazolin-4-yl)amino)pyridin-2-yl)-3-methyl-1H-imidazol-3-ium iodide phosphoric acid salt (Compound 43)
N-(6-(1H-1,2,4-triazol-1-yl)pyridin-3-yl)-7-chloroquinolin-4-amine (Compound 44)
7-chloro-N-(2-((dimethylamino)methyl)pyridin-4-yl)quinolin-4-amine (Compound 45)

As described in Examples, such pharmaceutical compositions contain at least one compound that inhibit interactions between GBAPs and HS-GAGs and therefore, such pharmaceutical compositions may be useful for treatment of amyloid and neurodegenerative diseases.

According to another embodiment, the present disclosure provides a method for the treatment and/or prevention of an amyloid disease, disorder or condition, comprising: selecting a subject in need of treating and/or preventing of an amyloid disease, disorder or condition, administering to a subject in need thereof a therapeutic amount of a pharmaceutical composition comprising at least one compound that inhibits binding of a GAG-binding amyloid peptide (GBAP) to a HS-GAG, of formula I, wherein the therapeutic amount is effective to inhibit binding of a GBAP to HS-GAG, wherein the amyloid disease, disorder or condition is treated or prevented.

According to one embodiment, the present disclosure provides a method of treating and/or preventing a neurodegenerative or amyloid disorder comprising: selecting a subject in need of treating and/or preventing a neurodegenerative or amyloid disorder, administering to a subject in need thereof a therapeutic amount of a compound that directly binds to a GAG, or to a HS-GAG, thereby treating and/or preventing the neurodegenerative or amyloid disorder.

The present disclosure provides assays enabling identification of small molecule compounds that bind directly and stably to GAGs, and are capable to inhibit GBAP binding to GAGs which are described in Examples.

Also provided are methods of screening compounds to identify inhibitor compounds of GBAP interaction with a GAG such as HS-GAG.

According to one embodiment, this disclosure provides a method of screening for small organic molecules that directly bind to GAGs and inhibit the binding of GAG-binding amyloid peptides (GBAPs) to GAGs, the method comprising the steps of:
(a) immobilizing a GAG to a surface of a multi-well plate
(b) contacting immobilized GAG with a known quantity of GBAP in the presence of at least one candidate compound; and
(c) measuring the amount of the GBAP bound to the GAG, wherein a significant decrease in GAG-GBAP binding as compared to GAG-GBAP binding in the absence of the candidate compound, identifies said compound as inhibitor of the GAG-GBAP interaction.

According to one embodiment, the GBAP is selected from a group consisting of beta-amyloid, tau, alpha-synuclein, TDP-43, IAPP and SAA. Examples of such assay are given in Examples.

The disclosure provides a compound of the general formula I:

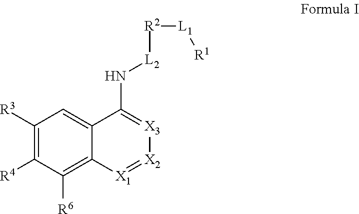

and a pharmaceutically acceptable diluent or carrier, wherein X₁, X₂, and X₃ are independent and optionally —CR⁹— or N; R⁹ is alkyl, halogen, —O-alkyl. If X₁ is CR⁹ then R⁹ and R⁶ can be joined to form a 5-7 member carbocyclic or heterocyclic ring; R³ and R⁴ are independent and optionally H, halogen, —O-alkyl. R³ and R⁴ can be joined to form a fused phenyl; R⁵ is H, C1-6 alkyl; R² is 5 or 6 member heterocycle or heteroaromatic optionally substituted with C1-6 alkyl, —OH, —O-alkyl, halogen; L₁ is a bond, —CH3, —CH2-, —[CR7R8]n- where R7 and R8 are independently: F, C1-6 alkyl, joined to form a C3-6 carbocycle or heterocycle n=0, 1, 2, 3; L₂ can be a bond or —N═C(R5)-; R1 is null, a 5 or 6-member heterocycle optionally substituted with alkyl, —O-alkyl, halogen; —N+(CH3)3, optionally substituted 4, 5 and 6-member heterocycle including heterocycles and heteroaromatics that may contain a quaternized nitrogen to form charges species; R₆ is alkyl; and pharmaceutically acceptable salts thereof

The disclosure provides a pharmaceutical composition comprising a therapeutic amount of a compound of formula I:

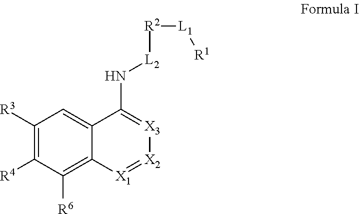

and a pharmaceutically acceptable diluent or carrier,
wherein X₁, X₂, and X₃ are independent and optionally —CR⁹— or N; R⁹ is alkyl, halogen, —O-alkyl. If X₁ is CR⁹ then R⁹ and R⁶ can be joined to form a 5-7 member carbocyclic or heterocyclic ring; R³ and R⁴ are independent and optionally H, halogen, —O-alkyl. R3 and R4 can be joined to form a fused phenyl; R⁵ is H, C1-6 alkyl; R² is 5 or 6 member heterocycle or heteroaromatic optionally substituted with C1-6 alkyl, —OH, —O-alkyl, halogen; L₁ is a bond, —CH3, —CH2-, —[CR7R8]n- where R7 and R8 are independently: F, C1-6 alkyl, joined to form a C3-6 carbocycle or heterocycle n=0, 1, 2, 3; L₂ can be a bond or —N═C(R⁵)—; R₁ is null, a 5 or 6-member heterocycle optionally substituted with alkyl, —O-alkyl, halogen; —N+(CH3)3, optionally substituted 4, 5 and 6-member heterocycle including heterocycles and heteroaromatics that may contain a quaternized nitrogen to form charges species; R₆ is alkyl; wherein the therapeutic amount is effective to inhibit binding of a GAG-binding amyloid peptide (GBAP) to a glycosaminoglycan (GAG). The disclosure provides a pharmaceutical composition wherein the compound of formula I is selected from the compounds as disclosed herein. The disclosure provides a pharmaceutical composition for the treatment or prevention of neurodegenerative diseases, disorders or conditions. The disclosure provides a pharmaceutical composition wherein the neurodegenerative disease, disorder or condition is Parkinson's Disease, Multiple System Atrophy or an alpha-synucleinopathy. The disclosure provides a pharmaceutical composition wherein the neurodegenerative disease, disorder or condition is Amyotrophic Lateral Sclerosis. The disclosure provides a pharmaceutical composition wherein the neurodegenerative disease, disorder or condition is Alzheimer's Disease or a dementia. The disclosure provides a pharmaceutical composition for the treatment or prevention of an amyloid disease, disorder, or condition. The disclosure provides a pharmaceutical composition wherein the amyloid disease is AA amyloidosis. The disclosure provides a pharmaceutical composition wherein the GBAP is beta-amyloid and the amyloid disease, disorder or condition is Alzheimer's Disease or Cerebral Amyloid Angiopathy. The disclosure provides a pharmaceutical composition wherein the GBAP is tau and the amyloid disease, disorder or condition wherein the amyloid neurodegenerative disorder is Parkinson's Disease, Multiple System Atrophy, an alpha-synucleinopathy, Alzheimer's Disease, Cerebral Amyloid Angiopathy, tauopathy, Frontotemporal Dementia, ALS, or AA amyloidosis. The disclosure provides a pharmaceutical composition wherein the GBAP is SAA and the amyloid disease, disorder or condition is AA amyloidosis.

The disclosure provides a method for the treatment or prevention of an amyloid disease, disorder or condition associated with GBAP binding to HS-GAGs comprising: selecting a subject in need of the treatment or prevention of an amyloid disease, disorder or condition associated with GBAP binding to HS-GAGs; administering to a subject in need thereof a therapeutic amount of a pharmaceutical composition comprising at least one compound of formula I that inhibits binding of a GAG-binding amyloid peptide (GBAP) to a glycosaminoglycan (GAG), wherein the compound is as disclosed herein, wherein the amyloid disease, disorder or condition associated with GBAP binding to HS-GAGs is treated or prevented in the subject.

The disclosure provides a method wherein the amyloid disease, disorder or condition wherein the amyloid neurodegenerative disorder is Parkinson's Disease, Multiple System Atrophy, an alpha-synucleinopathy, Alzheimer's Disease, Cerebral Amyloid Angiopathy, tauopathy, Frontotemporal Dementia, ALS, or AA amyloidosis. The disclosure provides a method wherein the compound is as disclosed herein.

The disclosure provides a method for the treatment or prevention of an amyloid disease, disorder or condition associated with an amyloid peptide binding to HS-GAGs, comprising: selecting a patient in need of the treatment or prevention of an amyloid disease, disorder or condition associated with an amyloid peptide binding to HS-GAGs; administering to a subject in need thereof a therapeutic amount of a pharmaceutical composition comprising at least one small molecule compound that inhibits binding of a GAG-binding amyloid peptide (GBAP) to a glycosaminoglycan (GAG), wherein the therapeutic amount is effective to inhibit binding of a GBAP to a GAG, further wherein the amyloid disease, disorder or condition associated with an amyloid peptide binding to HS-GAGs is treated or prevented in the patient. The disclosure provides a method wherein the compound that inhibits binding of a GAG-binding amyloid peptide (GBAP) to a glycosaminoglycan (GAG) is not a peptide or protein. The disclosure provides a method wherein the amyloid disease, disorder or condition is Parkinson's Disease, Multiple System Atrophy, an alpha-synucleinopathy, Alzheimer's Disease, Cerebral Amyloid Angiopathy, tauopathy, Frontotemporal Dementia, ALS, or AA amyloidosis. The disclosure provides a method wherein the compound is as disclosed herein.

The disclosure provides a method for detecting a small molecule compound that directly binds to a glycosaminoglycan (GAG) comprising: (a) immobilizing a GAG to a surface of a multi-well plate; (b) contacting immobilized GAG with a known quantity of GBAP in the presence of at least one candidate compound; and (c) measuring the amount of the GBAP bound to the immobilized GAG, wherein the small organic molecule compound is effective to inhibit binding of the GBAP to the GAG.

The disclosure provides a method for detecting a small organic molecule that directly binds to a glycosaminoglycan (GAG) comprising: (a) immobilizing a GAG to a surface of a multi-well plate (b) contacting a GAG with at least one candidate small organic compound; (c) removing unbound small organic compound; (d) adding a GBAP; and (e) measuring the amount of the GBAP bound to the immobilized GAG, wherein the small molecule compound is effective to inhibit binding of the GBAP to the GAG.

The disclosure provides a method wherein the GBAP is selected from the group consisting of amyloid-beta, alpha-synuclein, TDP-43, tau, SAA, IAPP, and derivatives and fragments thereof.

The disclosure provides a method for treatment or prevention of an amyloid neurodegenerative disorder comprising: selecting a subject in need of the treatment or prevention of an amyloid neurodegenerative disorder; administering to the subject a therapeutic amount of a compound that is a dual inhibitor of GBAP binding to a GAG, wherein the two GBAPs are selected from the group consisting of Abeta, tau, TDP-43 and alpha-synuclein, wherein the amyloid neurodegenerative disorder is treated or prevented in the subject. The disclosure provides a method wherein the two GBAPs are Abeta and tau, wherein the amyloid neurodegenerative disorder is Alzheimer's Disease. The disclosure provides a method wherein the GBAPs are tau and TDP-43 and the amyloid disorder is FTD. The disclosure provides a method wherein the amyloid neurodegenerative disorder is Parkinson's Disease, Multiple System Atrophy, an alpha-synucleinopathy, Alzheimer's Disease, Cerebral Amyloid Angiopathy, tauopathy, Frontotemporal Dementia, ALS, or AA amyloidosis. The disclosure provides a method wherein the compound is as disclosed.

The disclosure provides a method of treating an amyloid disorder comprising: selecting a subject in need of treatment of an amyloid disorder; administering to a subject in need thereof a therapeutic amount of a compound that binds to a glycosaminoglycan (GAG), wherein the therapeutic amount is effective to inhibit the GAG-binding amyloid peptide (GBAP) binding to the GAG, wherein the amyloid disorder is treated in the subject. The disclosure provides a method wherein the GAG is a heparan sulfate GAG (HS-GAG). The disclosure provides a method wherein the amyloid neurodegenerative disorder is Parkinson's Disease, Multiple System Atrophy, an alpha-synucleinopathy, Alzheimer's Disease, Cerebral Amyloid Angiopathy, tauopathy, Frontotemporal Dementia, ALS, or AA amyloidosis. The disclosure provides a method wherein the compound is as disclosed.

The disclosure provides for the use of the compositions of the disclosure for the production of a medicament for preventing and/or treating the indications as set forth herein.

In accordance with a further embodiment, the present disclosure provides a use of the pharmaceutical compositions described above, in an amount effective for use in a medicament, and most preferably for use as a medicament for treating a disease or disorder, for example, as set forth in herein, in a subject.

In accordance with yet another embodiment, the present disclosure provides a use of the pharmaceutical compositions described herein, and at least one additional therapeutic agent, in an amount effective for use in a medicament, and most preferably for use as a medicament for treating a disease or disorder associated with disease, for example, as set forth herein, in a subject.

The disclosure provides a method for treating and/or preventing a disease or condition as set forth herein in a patient, wherein said method comprises: selecting a patient in need of treating and/or preventing said disease or condition as set forth herein; administering to the patient a composition of the disclosure in a therapeutically effective amount, thereby treating and/or preventing said disease in said patient.

### BRIEF DESCRIPTION OF THE DRAWINGS

FIG. 1 shows inhibition curve of alpha-synuclein binding to immobilized heparin by Inhibitor Compounds 7 and 34.

FIG. 2 shows inhibition curve of Beta-amyloid (1-42) binding to purified human brain membranes by Inhibitor Compounds 22 and 24

FIG. 3 shows inhibition curve of Tau binding to immobilized heparin by Inhibitor Compounds 3 and 6.

### DETAILED DESCRIPTION OF THE INVENTION

The present disclosure describes compounds and pharmaceutical compositions for treatment of neurodegenerative and amyloid diseases, also described methods of treating neurodegenerative and amyloid disorders based on inhibiting glycosaminoglycan (GAG) interactions with amyloid peptides.

### Definitions

In accordance with the present invention and as used herein, the following terms are defined with the following meanings, unless explicitly stated otherwise.

As used in this specification and the appended claims, the singular forms “a”, “an” and “the” include the plural unless the context clearly dictates otherwise.

The terms “compound”, “small molecule compound”, “small organic compound”, “small organic molecule”, and “small molecule” are used interchangeably herein to refer to small organic molecule having a molecular weight less than 1200 Daltons and preferably between 200 Daltons to 800 Daltons. Such small molecule compound has no amino acids, no peptide bonds and does not contain carbohydrate. Such small molecule compounds are typically prepared by organic chemical synthesis.

The term “glycosaminoglycan” or “GAG” refers to sulfated glycosaminoglycans, including heparan sulfate (that is referred to in the art also as HS-GAG), heparin, chondroitin sulfate, dermatan sulfate and keratan sulfate. The term includes fragments of GAGs such as those that may be produced chemically, enzymatically or during purification. GAG may be free or attached to a linker, support, cell membrane, cell or protein, or otherwise chemically or enzymatically modified. GAGs may be crude or purified from organs, tissues or cells. The term does not include non-sulfated GAGs such as hyaluronan.

The term “proteoglycan” refers to heparan sulfate proteoglycans, chondroitin sulfate proteoglycans, dermatan sulfate proteoglycans and keratan sulfate proteoglycans. Proteoglycans have typically covalently attached GAGs. Examples are agrin, perlecan or versican.

The term “heparan sulfate” or “heparan sulfate glycosaminoglycan” or “HS-GAG” refers to heparan sulfate glycosaminoglycan. It includes fragments of heparan sulfate such as those that may be produced chemically, enzymatically or during purification. HS-GAG may be free or attached to a linker, support, cell or protein, or otherwise chemically or enzymatically modified. HS-GAGs may be crude or purified from organs, tissues or cells.

“Heparin” is a subtype of HS-GAG that is highly sulfated. It is a polysulfated polysaccharide, with no protein associated with it. As used herein, heparin refers to heparin prepared from different organs or species such as porcine intestinal mucosa heparin. It includes low molecular weight heparins, such as commercially available Fraxiparin, and other heparin derivatives, prepared or modified by chemical or enzymatic reaction.

The term “Glycosaminoglycan-Binding Amyloid Peptide” or “GAG-binding Amyloid Peptide” or “GBAP” or “GBAPs” refers to amyloid peptides and amyloid proteins that typically have a heparin-binding domain. GBAP binding to GAGs is typically stable under physiological conditions and results in acceleration of aggregation and fibrillogenesis. The list of GBAPs includes but is not limited to, Alzheimer's amyloid precursor protein (APP), beta-amyloid peptides (derived from APP) including beta-amyloid (1-42) and beta-amyloid (1-40), prion protein, Tau, alpha-synuclein, TDP-43, superoxide dismutase type 1 (SOD), IAPP, pro-IAPP, transthyretin, beta 2 microglobulin, immunoglobulin light chain AL, apolipoprotein Al, serum amyloid A (SAA), alpha-microglobulin, gelsolin, lysozyme, atrial natriuretic factor, calcitonin, medin and prion protein. The definition includes also peptide fragments, analogs, fusion proteins, derivatives and mutants.

The term “Inhibitor Compound” refers to a small molecule compound inhibiting the interaction between two molecules: (1) a GAG, exemplified by, but not restricted to heparin or HS-GAG and (2) a protein or peptide, exemplified by a GBAP. Inhibitor Compound is a small organic molecule having a molecular weight less than 1200 Daltons and preferably between 200 Daltons to 800 Daltons. Such small molecule compound has typically no amino acids, no peptide bonds and does not contain carbohydrate. Such small molecule compounds are typically prepared by organic chemical synthesis.

The term “synthetic chemical compound collection” or “compound collection” refers to a collection of random and semi-random synthetic small molecule compounds wherein each member of such collection or library is produced by chemical synthesis.

The term “beta-amyloid” or “Beta-Amyloid” or “amyloid-beta” or “Abeta” or “O-amyloid” refers to peptides that are involved in Alzheimer's Disease as the main component of amyloid plaques. The peptides result from proteolytic cleavage of Amyloid Precursor Protein (APP). Included are beta-amyloid peptides of 36-43 amino acids, beta-amyloid (1-40) (abbreviated also Abeta40) and beta-amyloid (1-42) (abbreviated also Abeta42) as well as other suitable peptide fragments, mutants, derivatives or fusions. Beta-amyloid peptides have a heparin binding domain.

The term “Tau” or “tau” refers to Tau and its peptide fragments. Tau is a microtubule binding protein that promotes microtubule assembly and stability. Tau is found to be the major component of the paired helical filaments (PHFs) found in the brains of patients with Alzheimer disease (AD). Tau is hyperphosphorylated in PHIFs. Tau and its bioactive fragments can be purified or produced by recombinant DNA technologies and are available commercially. Included are Tau proteins and Tau synthetic peptides that possess heparin-binding domain; many of Tau proteins and peptides are available from commercial suppliers.

The term “Alpha-Synuclein” or “α-Synuclein” refers to a 14 kD (140 amino acids) acidic presynaptic protein and peptide fragments. Alpha-synuclein is a major component of Parkinson's disease aggregates and is implicated in the pathogenesis of Parkinson's Disease and related neurodegenerative disorders. alpha-Synuclein accumulates in the brains of sporadic Parkinson's disease patients as a major component of Lewy bodies, which are intraneuronal cytoplasmic inclusions characteristic of Parkinson's disease. Included in the definition are alpha-synucleins and peptide fragments of alpha-synuclein, that possess a heparin binding domain—many of which are commercially available.

The term “TDP-43” refers to TAR DNA-binding protein 43 and its peptide fragments. A hyper-phosphorylated, ubiquitinated and cleaved form of TDP-43, known as pathologic TDP43, is the major disease protein in ubiquitin-positive, tau-, and alpha-synuclein-negative frontotemporal dementia (FTD) and in amyotrophic lateral sclerosis (ALS). The accumulation of TDP-43 aggregates in the central nervous system is a common feature of many neurodegenerative diseases, such as ALS, FTD, Alzheimer's disease (AD), and limbic predominant age-related TDP-43 encephalopathy (LATE).

The term “SAA” refers to Serum amyloid A and its peptide fragments. Serum amyloid A proteins are a family of apolipoproteins associated with high-density lipoprotein (HDL) in plasma. There are different isoforms of SAA. SAAs are implicated in several chronic diseases, such as amyloidosis, atherosclerosis, and rheumatoid arthritis. SAA, an acute phase plasma protein, is deposited extracellularly as insoluble amyloid fibrils that damage tissue structure and function.

The term “treatment” or “treating” is intended to include the administration of the compound of the invention for purposes which may include prophylaxis, amelioration, prevention or cure of disorders. Such treatment need not necessarily completely ameliorate the condition.

“Pharmaceutically acceptable excipient” means an excipient that is conventionally useful in preparing a pharmaceutical composition that is generally safe, non-toxic, and desirable, and includes excipients that are acceptable for veterinary use as well as for human pharmaceutical use. Such excipients may be solid, liquid, semisolid, or, in the case of an aerosol composition, gaseous.

“Pharmaceutically acceptable salt” means a salt that is pharmaceutically acceptable and have the desired pharmacological properties. Such salts include salts that may be formed where acidic protons present in the compounds are capable of reacting with inorganic or organic bases. Suitable inorganic salts include those formed with the alkali metals, e.g. sodium and potassium, magnesium, calcium, and aluminum. Suitable organic salts include those formed with organic bases such as the amine bases, e.g. ethanolamine, diethanolamine, triethanolamine, tromethamine, N-methylglucamine, and the like. Such salts also include acid addition salts formed with inorganic acids (e.g. hydrochloric and hydrobromic acids) and organic acids (e.g. acetic acid, citric acid, maleic acid, and the alkane- and arene-sulfonic acids such as methanesulfonic acid and benzenesulfonic acid). When there are two acidic groups present, a pharmaceutically acceptable salt may be a mono-acid-mono-salt or a di-salt; and similarly where there are more than two acidic groups present, some or all of such groups can be salified.

A “therapeutically effective amount” in general means the amount that is sufficient to affect the desired degree of treatment for the disease. A “therapeutically effective amount” or a “therapeutically effective dosage” preferably diminishes signs of amyloidosis or treats a disease associated with these conditions, such as an amyloid disease or a synucleinopathy, by at least 20%, more preferably by at least 40%, even more preferably by at least 60%, and still more preferably by at least 80%, relative to an untreated subject. A broad range of disclosed composition dosages are believed to be both safe and effective.

“Treating” or “treatment” of a disease includes preventing the disease from occurring in a mammal that may be predisposed to the disease but does not yet experience or exhibit symptoms of the disease (prophylactic treatment), inhibiting the disease (slowing or arresting its development), providing relief from the symptoms or side-effects of the disease (including palliative treatment), and relieving the disease (causing regression of the disease), such as by disruption of pre-formed amyloid or synuclein fibrils. One such preventive treatment may be use of the disclosed compounds for the treatment of Mild Cognitive impairment (MCI).

“Disruption of fibrils or fibrillogenesis” refers to the disruption of pre-formed amyloid or synuclein fibrils, that usually exist in a pre-dominant β-pleated sheet secondary structure. Such disruption by compounds of the invention may involve marked reduction or disassembly of amyloid or synuclein fibrils as assessed by various methods such as circular dichroism spectroscopy, Thioflavin T fluorometry, Congo red binding, SDS-PAGE/Western blotting, as demonstrated by the Examples presented in this application.

As used herein the term “active pharmaceutical ingredient” (“API”) or “pharmaceutically active agent” is a drug or agent which can be employed as disclosed herein and is intended to be used in the human or animal body in order to heal, to alleviate, to prevent or to diagnose diseases, ailments, physical damage or pathological symptoms; allow the state, the condition or the functions of the body or mental states to be identified; to replace active substances produced by the human or animal body, or body fluids; to defend against, to eliminate or to render innocuous pathogens, parasites or exogenous substances or to influence the state, the condition or the functions of the body or mental states. Drugs in use can be found in reference works such as, for example, the Rote Liste or the Merck Index.

“A pharmaceutical agent” or “pharmacological agent” or “pharmaceutical composition” refers to a compound or combination of compounds used for treatment, preferably in a pure or near pure form. In the specification, pharmaceutical or pharmacological agents include the compounds of this invention. The compounds are desirably purified to 80% homogeneity, and preferably to 90% homogeneity. Compounds and compositions purified to 99.9% homogeneity are believed to be advantageous. As a test or confirmation, a suitable homogeneous compound on HPLC would yield, what those skilled in the art would identify as a single sharp-peak band.

As used herein, the terms “subject” and “patient” are used interchangeably. As used herein, the term “patient” refers to an animal, preferably a mammal such as a non-primate (e.g., cows, pigs, horses, cats, dogs, rats etc.) and a primate (e.g., monkey and human), and most preferably a human. In some embodiments, the subject is a non-human animal such as a farm animal (e.g., a horse, pig, or cow) or a pet (e.g., a dog or cat). In a specific embodiment, the subject is an elderly human. In another embodiment, the subject is a human adult. In another embodiment, the subject is a human child. In yet another embodiment, the subject is a human infant.

“Amyloid disease” refers to any of a number of disorders which have as a symptom or as part of its pathology the accumulation or formation of protein aggregates. “Disorders and/or diseases”, “disorder(s)” and “disease(s)” are used interchangeably herein and include a condition characterized by abnormal protein folding or aggregation or abnormal amyloid formation, deposition, accumulation or persistence, or amyloid lipid interactions. In some aspects, the term includes conditions characterized by abnormal protein folding or aggregation or amyloid formation, deposition, accumulation or persistence. According to some embodiments, the disease is a condition of the central or peripheral nervous system or systemic organ. According to some embodiments, the terms include conditions associated with protein misfolding; the formation, deposition, accumulation, or persistence of amyloid or amyloid fibrils, comprising an amyloid protein comprising or selected from the group consisting of beta-amyloid, tau, alpha-synuclein, TDP-43, SAA, SOD, AA amyloid, AL amyloid, IAPP, PrP amyloid, alpha2-microglobulin amyloid, transthyretin, prealbumin, and procalcitonin. A disorder and/or disease may be a condition where it is desirable to dissociate abnormally aggregated proteins and/or dissolve or disrupt pre-formed or pre-deposited amyloid or amyloid fibril and/or prevent cellular accumulation and/or spreading in tissues of misfolded or aggregated proteopathic seeds.

“Amyloid Cell” or “Cellular Amyloidosis” is defined as a cell which displays signs of amyloidosis phenotype. By way of example, these signs may include indications of amyloid peptide or protein aggregation, accumulation of amyloid aggregates inside cells, etc. Typically such signs may be detectable by biochemical or histological methods and may involve also signs of cytotoxicity such as affecting lysosomal appearance.

“Amyloidosis” refers to signs of an amyloid protein aggregation and amyloid fibril formation, typically associated with toxicity. Other related terms are protein conformational disorders, protein misfolding diseases, proteopathies and proteinopathies. Amyloidosis refers to a diverse group of diseases of acquired or hereditary origin and characterized by the accumulation of one of several different types of protein fibrils with similar properties called amyloid. Amyloid can accumulate in a single organ or be dispersed throughout the body. The disease can cause serious problems in the affected areas, which may include the heart, brain, kidneys and digestive tract. The fibrillar composition of amyloid deposits is an identifying characteristic for various amyloid diseases. Intracerebral and cerebrovascular deposits composed primarily of fibrils of beta-amyloid peptide are characteristic of Alzheimer's disease (both familial and sporadic forms); islet amyloid protein peptide (IAPP; amylin) is characteristic of the fibrils in pancreatic islet cell amyloid deposits associated with type II diabetes; and, beta-2-microglobulin is a major component of amyloid deposits which form as a consequence of long term hemodialysis treatment. Prion-associated diseases, such as Creutzfeld-Jacob disease, scrapie, bovine spongiform encephalitis, and the like are characterized by the accumulation of a protease-resistant form of a prion protein.

In other aspects of the invention, disorders and/or diseases that can be treated and/or prevented using the compounds, compositions and methods of the invention include conditions of the central or peripheral nervous system or a systemic organ that result in the deposition of proteins, protein fragments, and peptides in beta-pleated sheets, fibrils, and/or aggregates or oligomers. According to some embodiments, the disease is Alzheimer's disease, presenile and senile forms; amyloid angiopathy; mild cognitive impairment; Alzheimer's disease-related dementia (e.g., vascular or Alzheimer dementia).

The terms “treatment”, “treating”, “treat” and the like are used herein to generally mean obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. “Treatment” as used herein covers any treatment of a disease in a mammal, for example, a human, and includes:
(a) preventing the disease or symptom from occurring in a subject which may be predisposed to the disease or symptom but has not yet been diagnosed as having it;
(b) inhibiting the disease symptom, i.e., arresting its development; or
(c) relieving the disease symptom, i.e., causing regression of the disease or symptom.

By “effective dose” or “amount effective” is meant administration of a compound sufficient to provide the desired physiological and/or psychological change. This will vary depending on the patient, the disease and the treatment. The dose may either be a therapeutic dose, in which case it should sufficiently alter levels of amyloid plaques in the subject to alleviate or ameliorate the symptoms of the disorder or condition, or a prophylactic dose, which should be sufficient to prevent accumulation of amyloid plaques to an undesirable level.

The term “diagnosis” is used herein to cover any type of analysis used to determine or project a status which includes identification of a disease from its symptoms and determining the presence of molecules (e.g., apoE) in an area (e.g., brain tissue) which suggest a disease status (e.g., beginnings of Alzheimer's disease).

The term “unit dosage form,” as used herein, refers to physically discrete units suitable as unitary dosages for human and animal subjects, each unit containing a predetermined quantity of compounds of the present invention calculated in an amount sufficient to produce the desired effect in association with a pharmaceutically acceptable diluent, carrier or vehicle. The specifications for the described unit dosage forms of the present invention depend on the compound employed and the effect to be achieved, and the pharmacodynamics associated with each compound in the host.

The term “Alzheimer's disease” (abbreviated herein as “AD”) as used herein refers to a irreversible, progressive brain disorder that slowly destroys memory and thinking skills. Alzheimer's disease is a type of dementia thought to be caused by the abnormal build-up of proteins in and around brain cells. One of the proteins involved is called beta-amyloid, deposits of which form plaques around brain cells. The other protein is called tau, deposits of which form tangles within brain cells. AD is associated with formation of neuritic plaques comprising amyloid-beta protein primarily in the hippocampus and cerebral cortex, as well as impairment in both learning and memory. “AD” as used herein is meant to encompass both AD as well as AD-type pathologies. The term “AD-type pathology” as used herein refers to a combination of CNS alterations including, but not limited to, formation of neuritic plaques containing amyloid p protein in the hippocampus and cerebral cortex.

The term “cerebral amyloid angiopathy” (abbreviated herein as CAA) as used herein refers to a condition associated with formation of beta-amyloid deposition within cerebral vessels which can be complicated by cerebral parenchymal hemorrhage. CAA can also be associated with dementia prior to onset of hemorrhages. The vascular amyloid deposits associated with CAA can exist in the absence of AD, but are more frequently associated with AD.

The term “synucleinopathy” or “α-Synucleinopathy” or “alpha-synucleinopathy” refers to neurodegenerative diseases characterized by the abnormal accumulation of aggregates of alpha-synuclein protein in neurons, nerve fibres or glial cells. There are three main types of synucleinopathy: Parkinson's disease (PD), dementia with Lewy bodies (DLB), and multiple system atrophy (MSA). Other rare disorders, such as various neuroaxonal dystrophies, also have α-synuclein pathologies.

The term “tauopathy” or “Tauopathy” refers to a class of neurodegenerative diseases involving the aggregation of tau protein into neurofibrillary or gliofibrillary tangles (NFTs) in the human brain. Tangles are formed by hyperphosphorylation of the microtubule protein known as tau, causing the protein to dissociate from microtubules and form insoluble aggregates. These aggregations are also called paired helical filaments. Examples of tauopathies are Alzheimer's Disease, Progressive supranuclear palsy (PSP), Frontotemporal dementia and parkinsonism linked to chromosome 17 (FTDP-17).

The term “beta-amyloid deposit” as used herein refers to a deposit in the brain composed of beta-amyloid as well as other substances. The term “prion” shall mean an infectious particle known to cause diseases (spongiform encephalopathies) in humans and animals. The term “prion-mediated disorder” refers to any disorder caused by infection with a prion particle. Known prions include those which infect animals to cause scrapie, a transmissible, degenerative disease of the nervous system of sheep and goats as well as bovine spongiform encephalopathies (BSE) or mad cow disease and feline spongiform encephalopathies of cats.

The term “alkyl” as defined herein, alone or in combination, typically refers to a straight or branched alkyl radical, preferably having 1-6 carbon atoms, i.e., (C1-C6)alkyl, and includes, for example, methyl, ethyl, n-propyl, isopropyl, n-butyl, isobutyl, tert-butyl, n-pentyl, isopentyl, 2,2-dimethylpropyl, and n-hexyl.

The term “aryl”, alone or in combination, refers to an aromatic carbocyclic group preferably of 6 to 20, more preferably 6 to 10 carbon atoms, i.e., (C6-C20) or (C6-C10) aryl, respectively, such as phenyl and naphthyl.

The term “heterocyclyl” or “hetercycle” or “heterocyclic, alone or in combination, refers to a radical derived from a mono- or poly-cyclic ring containing one to three heteroatoms selected from the group consisting of N, O and S, with or without unsaturation or aromatic character. The term “heteroaryl” refers to such a mono- or poly-cyclic ring having aromatic character. Any alkyl, cycloalkyl, aryl, heteroaryl or heterocyclyl radical may be substituted by one or more radicals including, but not limited to, halogen, hydroxy, (C1-C10) alkyl, (C2-C10) alkenyl, (C2-C10) alkynyl, (C7-C12) aralkyl, (C6-C10) aryl, (C7-C12) alkaryl, (C1-C10) alkoxy, (C6-C10) aryloxy, (C1-C10) alkylthio, (C6-C10) arylthio, (C6-C10) arylamino, (C3-C10) cycloalkyl, (C3-C10) cycloalkenyl, amino, (C1-C10) alkylamino, di(C1-C10)-alkylamino, (C2-C12) alkoxyalkyl, (C2-C12) alkylthio-alkyl, (C1-C10) alkylsulfinyl, (C1-C10) alkylsulfonyl, (C6-C10) arylsulfonyl, hydroxy-(C1-C10)alkyl, (C6-C10)aryloxy(C1-C10)alkyl, (C1-C10)alkoxycarbonyl, (C6-C10)aryl-oxycarbonyl, (C2-C11) alkanoyl, (C7-C11)aroyl, fluoro(C1-C10)alkyl, oxo, nitro, nitro-(C1-C10)alkyl, cyano, cyano(C1-C10)alkyl, aminocarbonyl, (C1-C10)alkyl-aminocarbonyl, di(C1-C10)-alkylaminocarbonyl, aminocarbonyl(C1-C10)alkyl, aminocarbonyl(C6-C10)aryl, aminosulfonyl, (C1-C10)alkylaminosulfonyl, di(C1-C10)-alkylaminosulfonyl, amidino, carboxy, sulfo, heterocyclyl, and —(CH2)m-Z—(C1-C10 alkyl), where m is 1 to 8 and Z is oxygen or sulfur.

The term “halogen” refers to fluoro, chloro, bromo or iodo.

It is to be understood that the term “substituted”, as used herein, means that any one or more hydrogen on the designated atom is replaced with a selection from the indicated groups, provided that the designated atom's normal valency is not exceeded, and that the substitution results in a stable compound. Combinations of substituents are permissible only if such combinations result in stable compounds. By “stable compound” or “stable structure” it is meant herein a compound that is sufficiently robust to survive isolation to a useful degree of purity from a reaction mixture, and formulation into an efficacious therapeutic agent.

As contemplated herein, the present invention further encompasses isomers, including ²H (deuterium), ¹⁸F and ¹¹C, pharmaceutically acceptable salts and hydrates and solvates of the compounds defined by the present invention.

In addition, this invention further includes hydrates of the compounds described herein. The term “hydrate” includes, but is not limited to hemihydrate, monohydrate, dihydrate, trihydrate, and the like.

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges which may independently be included in the smaller ranges also are encompassed within the invention, subject to any specifically excluded limit in the stated range. Where the stated range includes one or both of the limits, ranges excluding either or both of those included limits are also included in the invention.

Abbreviations: APP: amyloid precursor protein; AD: Alzheimer's disease; CS-GAG: Chondroitin sulfate glycosaminoglycan; BSA: bovine serum albumin; GAGs: glycosaminoglycans; GBAP: GAG-binding amyloid peptide; HS-GAG: heparan sulfate glycosaminoglycan; HSPGs: heparan sulfate proteoglycans; DMSO: dimethylsulfoxide; ELISA—enzyme-linked immunoassay; Tris: tris (hydroxy-methyl) aminomethane; DS-GAG: dermatan sulfate; KS-GAG: keratan sulfate; SAR: Structure-Activity Relationship; NCE—New chemical entity; SAA: Serum amyloid A; TDP-43: TAR DNA-binding protein 43; SOD: Superoxide Dismutase 1; PD: Parkinson's Disease.

The present disclosure provides small molecule compounds that inhibit binding of GAG-binding amyloid peptides (GBAPs) to heparan sulfate glycosaminoglycans (HS-GAGs) and thus may be useful as therapeutics, for instance for Alzheimer's Disease as well as other amyloid and neurodegenerative diseases.

### Active Agents

According to one embodiment the disclosure provides active agents, such as, compound of a general formula I:

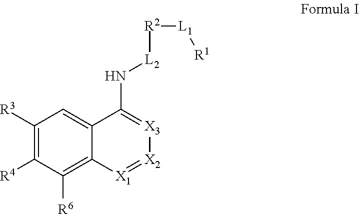

and a pharmaceutically acceptable diluent or carrier
   wherein
X₁, X₂, and X₃ are independent and optionally —CR⁹— or N; R⁹ is alkyl, halogen, —O-alkyl. If X₁ is CR⁹ then R⁹ and R⁶ can be joined to form a 5-7 member carbocyclic, heteroaromatic or heterocyclic ring;
R³ and R⁴ are independent and optionally H, halogen, —O-alkyl. R³ and R⁴ can be joined to form a fused phenyl, C1-7 carbocycle or heterocyclic or heteroaromatic ring;
R⁵ is H, C1-6 alkyl;
R² is phenyl optionally substituted with C1-6 alkyl, —OH, —O-alkyl, halogen, heterocycle, heteroaromatic; 5 or 6 member heterocycle or heteroaromatic optionally substituted with C1-6 alkyl, —OH, —O-alkyl, halogen; a 5 or 6-member heterocycle optionally substituted with alkyl, —O-alkyl, halogen, heterocycle, heteroaromatic; —N+(CH3)3, optionally substituted 4, 5 and 6-member heterocycle including heterocycles and heteroaromatics that may contain a quaternized nitrogen to form a charged species:
L₁ is a bond, —CH3, —CH2-, —[CR7R8]n- where R7 and R8 are independently: F, C1-6 alkyl, joined to form a C3-6 carbocycle or heterocycle or heteroaromatic
n=0, 1, 2, 3;
L₂ can be a bond or —N═C(R5)-;
R₁ is null, a 5 or 6-member heterocycle optionally substituted with alkyl, —O-alkyl, halogen; —N+(CH3)3, optionally substituted 4, 5 and 6-member heterocycle including heterocycles and heteroaromatics that may contain a quaternized nitrogen to form charges species;
R₆ is alkyl;
and pharmaceutically acceptable salts thereof. Also included are all geometrical isomers and tautomers of the nitrogen-carbon double bond.

Chemical synthesis of such compounds are described in Examples. Chemical structures are shown in Table 1. Analytical chemistry data of the compounds such as mass spectrometry and NMR data analysis are described in Examples.

As shown in Examples, the compounds of Formula I inhibit the interaction between GBAPs and HS-GAGs, and thus may be useful as therapeutics of neurodegenerative diseases associated with amyloidosis, and other amyloid diseases.

According to one embodiment, the compound of formula I that inhibits binding of a GBAP to HS-GAGs is selected from the group consisting of
(E)-4-((E)-(4-(1H-imidazol-1-yl)-2-methylbenzylidene)hydrazono)-7-chloro-1,4-dihydroquinoline (Compound 1)
(E)-7-chloro-4-((E)-(4-(4,5-dimethyl-1H-imidazol-1-yl)benzylidene)hydrazono)-1,4-dihydroquinoline (Compound 2)
(E)-4-((E)-(4-(1H-pyrazol-1-yl)benzylidene)hydrazono)-7-chloro-1,4-dihydroquinoline (Compound 3)
(Z)-1-(4-((2-(6-chloroquinolin-4-yl)hydrazineylidene)methyl)phenyl)—N,N,N-trimethylmethanaminium iodide (Compound 4)
(E)-4-(2-(4-(1H-imidazol-1-yl)benzylidene)hydrazinyl)-7-fluoroquinoline (Compound 5)
(E)-4-(4-((2-(7-fluoroquinolin-4-yl)hydrazono)methyl)benzyl)morpholine (Compound 6)
(E)-7-chloro-4-(2-(4-((4-methylpiperazin-1-yl)methyl)benzylidene)hydrazinyl)quinolone (Compound 7)
(E)-1-(4-((2-(1,2-dihydroacenaphthylen-5-yl)hydrazineylidene)methyl)phenyl)-1H-imidazole (Compound 8)
(Z)-1-methyl-4-((2-(quinolin-4-yl)hydrazono)methyl)pyridin-1-ium iodide (Compound 9)
(E)-4-(4-((2-(7-fluoroquinolin-4-yl)hydrazono)methyl)benzyl)thiomorpholine 1,1-dioxide (Compound 10)
4-(2-(4-(1H-imidazol-1-yl)-3-methylbenzylidene)hydrazineyl)-6,7-dimethoxyquinoline hydrochloride (Compound 11)
4-(2-(4-(1H-imidazol-1-yl)-3-methylbenzylidene)hydrazineyl)-7-methoxyquinoline (Compound 12)
(E)-4-(2-(4-(1H-imidazol-1-yl)-3-methoxybenzylidene)hydrazineyl)-6,7-dimethoxyquinoline phosphate (Compound 13)
(E)-4-(2-((6-(1H-imidazol-1-yl)pyridin-3-yl)methylene)hydrazineyl)-7-chloroquinoline hydrochloride (Compound 14)
7-chloro-4-(2-((6-(4,5-dimethyl-1H-imidazol-1-yl)pyridin-3-yl)methylene)hydrazineyl)quinazoline hydrochloride (Compound 15)
7-chloro-4-(2-(1-(6-(4,5-dimethyl-1H-imidazol-1-yl)pyridin-3-yl)ethylidene)hydrazineyl)quinazoline phosphate (Compound 16)
(E)-1-(5-(1-(2-(7-chloroquinazolin-4-yl)hydrazineylidene)ethyl)pyridin-2-yl)-3-methyl-1H-imidazol-3-ium iodide phosphoric acid salt (Compound 17)
(E)-7-chloro-4-(2-(1-(6-(2,4-dimethyl-1H-imidazol-1-yl)pyridin-3-yl)ethylidene)hydrazineyl)quinazoline phosphoric acid salt (Compound 18)
(E)-4-(2-(1-(6-(2,4-dimethyl-1H-imidazol-1-yl)pyridin-3-yl)ethylidene)hydrazineyl)quinazoline (Compound 19)
7-chloro-N-(2-(4-methylpiperazin-1-yl)pyridin-4-yl)quinolin-4-amine (Compound 20)
(E)-4-(2-(1-(6-(4,5-dimethyl-1H-imidazol-1-yl)pyridin-3-yl)ethylidene)hydrazineyl)-7-fluoroquinazoline (Compound 21)
7-chloro-4-(2-(1-(6-(4,5-dimethyl-1H-imidazol-1-yl)pyridin-3-yl)ethylidene)hydrazineyl)quinoline (Compound 22)
1-(5-(1-(2-(7-chloroquinolin-4-yl)hydrazineylidene)ethyl)pyridin-2-yl)-3-methyl-1H-imidazol-3-ium iodide (Compound 23)
(E)-6-chloro-1-(2-(1-(6-(2,4-dimethyl-1H-imidazol-1-yl)pyridin-3-yl)ethylidene)hydrazineyl)phthalazine (Compound 24)
7-chloro-N-(6-(4,5-dimethyl-1H-imidazol-1-yl)pyridin-3-yl)quinazolin-4-amine (Compound 25)
(E)-1-(5-(1-(2-(7-fluoroquinazolin-4-yl)hydrazineylidene)ethyl)pyridin-2-yl)-3-methyl-1H-imidazol-3-ium phosphoric acid salt (Compound 26)
(E)-7-chloro-4-(2-(1-(6-(2,4-dimethyl-1H-imidazol-1-yl)pyridin-3-yl)ethylidene)hydrazineyl)quinazoline hydrochloride (Compound 27)
7-chloro-4-(2-(1-(2-(4,5-dimethyl-1H-imidazol-1-yl)pyridin-4-yl)ethylidene)hydrazineyl)quinazoline (Compound 28)
1-(3-(1-(2-(7-chloroquinazolin-4-yl)hydrazineylidene)ethyl)phenyl)—N,N-dimethylmethanamine (Compound 29)
7-chloro-4-(2-(1-(3-(4-methylpiperazin-1-yl)phenyl)ethylidene)hydrazineyl)quinazoline (Compound 30)
6-chloro-N-(6-(4,5-dimethyl-1H-imidazol-1-yl)-5-methoxypyridin-3-yl)phthalazin-1-amine phosphoric acid salt (Compound 31)
(E)-4-(2-(1-(6-(1H-1,2,4-triazol-1-yl)pyridin-3-yl)ethylidene)hydrazineyl)-7-chloroquinazoline (Compound 32)
7-chloro-N-(6-(4,5-dimethyl-1H-imidazol-1-yl)-5-methoxypyridin-3-yl)isoquinolin-4-amine phosphoric acid salt (Compound 33)
N-(6-(1H-1,2,4-triazol-1-yl)pyridin-3-yl)-7-chloroquinazolin-4-amine phosphoric acid salt (Compound 34)
(E)-7-chloro-4-(2-(3-fluoro-4-(1H-imidazol-1-yl)benzylidene)hydrazineyl)quinoline hydrochloride (Compound 35)
7-chloro-N-(6-(2,4-dimethyl-1H-imidazol-1-yl)pyridin-3-yl)isoquinolin-4-amine phosphoric acid salt (Compound 37)
5-((7-chloroquinazolin-4-yl)amino)-2-(4,5-dimethyl-1H-imidazol-1-yl)pyridin-3-ol phosphoric acid salt (Compound 40)
N-(6-(1H-imidazol-1-yl)pyridin-3-yl)-7-chloroquinazolin-4-amine (Compound 41)
7-chloro-N-(3-((diethylamino)methyl)phenyl)quinolin-4-amine (Compound 42)
1-(5-((7-chloroquinazolin-4-yl)amino)pyridin-2-yl)-3-methyl-1H-imidazol-3-ium iodide phosphoric acid salt (Compound 43)
N-(6-(1H-1,2,4-triazol-1-yl)pyridin-3-yl)-7-chloroquinolin-4-amine (Compound 44)
7-chloro-N-(2-((dimethylamino)methyl)pyridin-4-yl)quinolin-4-amine (Compound 45)

These compounds have biological activities as described in Examples, and thus may be useful as therapeutics for neurodegenerative and other amyloid diseases.

According to one embodiment the present disclosure provides a pharmaceutical composition comprising a compound of general formula I:

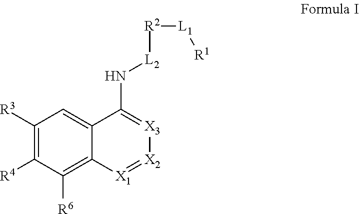

and a pharmaceutically acceptable diluent or carrier
wherein
X₁, X₂, and X₃ are independent and optionally —CR⁹— or N; R⁹ is alkyl, halogen, —O-alkyl. If X₁ is CR⁹ then R⁹ and R⁶ can be joined to form a 5-7 member carbocyclic, heteroaromatic or heterocyclic ring;
R³ and R⁴ are independent and optionally H, halogen, —O-alkyl. R³ and R⁴ can be joined to form a fused phenyl, C1-7 carbocycle or heterocyclic or heteroaromatic ring;
R⁵ is H, C1-6 alkyl;
R² is phenyl optionally substituted with C1-6 alkyl, —OH, —O-alkyl, halogen, heterocycle, heteroaromatic; 5 or 6 member heterocycle or heteroaromatic optionally substituted with C1-6 alkyl, —OH, —O-alkyl, halogen; a 5 or 6-member heterocycle optionally substituted with alkyl, —O-alkyl, halogen, heterocycle, heteroaromatic; —N+(CH3)3, optionally substituted 4, 5 and 6-member heterocycle including heterocycles and heteroaromatics that may contain a quaternized nitrogen to form a charged species:
L₁ is a bond, —CH3, —CH2-, —[CR7R8]n- where R⁷ and R⁸ are independently: F, C1-6 alkyl, joined to form a C3-6 carbocycle or heterocycle or heteroaromatic.
n=0, 1, 2, 3;
L₂ can be a bond or —N═C(R⁵)—;
R₁ is null, a 5 or 6-member heterocycle optionally substituted with alkyl, —O-alkyl, halogen; —N+(CH3)3, optionally substituted 4, 5 and 6-member heterocycle including heterocycles and heteroaromatics that may contain a quaternized nitrogen to form a charged species;
R₆ is alkyl;
and pharmaceutically acceptable salts thereof, the pharmaceutical composition being characterized by its ability to inhibit binding of a GAG-binding amyloid peptide (GBAP) to a HS-GAGs.

As described in Examples, such pharmaceutical compositions contain compounds that inhibit interactions between GBAPs and HS-GAGs and therefore, such pharmaceutical compositions may be useful for prevention or treatment of amyloid and neurodegenerative diseases.

According to another embodiment, a method of treating an amyloid disease or disorder is provided, said method comprising the step of administering to a subject in need thereof a therapeutically effective amount of a compound according to formula I, which compound is inhibiting GBAP binding to the HS-GAG. According to one embodiment, GBAP is beta-amyloid and the amyloid disorder is Alzheimer's Disease or Cerebral Amyloid Angiopathy. According to another embodiment, GBAP is alpha-synuclein and the amyloid disorder is Parkinson's Disease or a synucleinopathy. According to another embodiment, GBAP is Tau and the amyloid disorder is Alzheimer's Disease, Frontotemporal Dementia or a tauopathy. According to another embodiment, GBAP is TDP-43 and the amyloid disorder is ALS or another disease associated with TDP-43 amyloidosis. According to another embodiment, GBAP is SAA and the amyloid disease or disorder is associated with SAA amyloidosis.

According to one embodiment the pharmaceutical composition comprises an active agent compound of general formula I that inhibits binding of a GBAP to a GAG and is selected from the group consisting of
(E)-4-((E)-(4-(1H-imidazol-1-yl)-2-methylbenzylidene)hydrazono)-7-chloro-1,4-dihydroquinoline (Compound 1)
(E)-7-chloro-4-((E)-(4-(4,5-dimethyl-1H-imidazol-1-yl)benzylidene)hydrazono)-1,4-dihydroquinoline (Compound 2)
(E)-4-((E)-(4-(1H-pyrazol-1-yl)benzylidene)hydrazono)-7-chloro-1,4-dihydroquinoline (Compound 3)
(Z)-1-(4-((2-(6-chloroquinolin-4-yl)hydrazineylidene)methyl)phenyl)—N,N,N-trimethylmethanaminium iodide (Compound 4)
(E)-4-(2-(4-(1H-imidazol-1-yl)benzylidene)hydrazinyl)-7-fluoroquinoline (Compound 5)
(E)-4-(4-((2-(7-fluoroquinolin-4-yl)hydrazono)methyl)benzyl)morpholine (Compound 6)
(E)-7-chloro-4-(2-(4-((4-methylpiperazin-1-yl)methyl)benzylidene)hydrazinyl)quinolone (Compound 7)
(E)-1-(4-((2-(1,2-dihydroacenaphthylen-5-yl)hydrazineylidene)methyl)phenyl)-1H-imidazole (Compound 8)
(Z)-1-methyl-4-((2-(quinolin-4-yl)hydrazono)methyl)pyridin-1-ium iodide (Compound 9)
(E)-4-(4-((2-(7-fluoroquinolin-4-yl)hydrazono)methyl)benzyl)thiomorpholine 1,1-dioxide (Compound 10)
4-(2-(4-(1H-imidazol-1-yl)-3-methylbenzylidene)hydrazineyl)-6,7-dimethoxyquinoline hydrochloride (Compound 11)
4-(2-(4-(1H-imidazol-1-yl)-3-methylbenzylidene)hydrazineyl)-7-methoxyquinoline (Compound 12)
(E)-4-(2-(4-(1H-imidazol-1-yl)-3-methoxybenzylidene)hydrazineyl)-6,7-dimethoxyquinoline phosphate (Compound 13)
(E)-4-(2-((6-(1H-imidazol-1-yl)pyridin-3-yl)methylene)hydrazineyl)-7-chloroquinoline hydrochloride (Compound 14)
7-chloro-4-(2-((6-(4,5-dimethyl-1H-imidazol-1-yl)pyridin-3-yl)methylene)hydrazineyl)quinazoline hydrochloride (Compound 15)
7-chloro-4-(2-(1-(6-(4,5-dimethyl-1H-imidazol-1-yl)pyridin-3-yl)ethylidene)hydrazineyl)quinazoline phosphate (Compound 16)
(E)-1-(5-(1-(2-(7-chloroquinazolin-4-yl)hydrazineylidene)ethyl)pyridin-2-yl)-3-methyl-1H-imidazol-3-ium iodide phosphoric acid salt (Compound 17)
(E)-7-chloro-4-(2-(1-(6-(2,4-dimethyl-1H-imidazol-1-yl)pyridin-3-yl)ethylidene)hydrazineyl)quinazoline phosphoric acid salt (Compound 18)
(E)-4-(2-(1-(6-(2,4-dimethyl-1H-imidazol-1-yl)pyridin-3-yl)ethylidene)hydrazineyl)quinazoline (Compound 19)
7-chloro-N-(2-(4-methylpiperazin-1-yl)pyridin-4-yl)quinolin-4-amine (Compound 20)
(E)-4-(2-(1-(6-(4,5-dimethyl-1H-imidazol-1-yl)pyridin-3-yl)ethylidene)hydrazineyl)-7-fluoroquinazoline (Compound 21)
7-chloro-4-(2-(1-(6-(4,5-dimethyl-1H-imidazol-1-yl)pyridin-3-yl)ethylidene)hydrazineyl)quinoline (Compound 22)
1-(5-(1-(2-(7-chloroquinolin-4-yl)hydrazineylidene)ethyl)pyridin-2-yl)-3-methyl-1H-imidazol-3-ium iodide (Compound 23)
(E)-6-chloro-1-(2-(1-(6-(2,4-dimethyl-1H-imidazol-1-yl)pyridin-3-yl)ethylidene)hydrazineyl)phthalazine (Compound 24)
7-chloro-N-(6-(4,5-dimethyl-1H-imidazol-1-yl)pyridin-3-yl)quinazolin-4-amine (Compound 25)
(E)-1-(5-(1-(2-(7-fluoroquinazolin-4-yl)hydrazineylidene)ethyl)pyridin-2-yl)-3-methyl-1H-imidazol-3-ium phosphoric acid salt (Compound 26)
(E)-7-chloro-4-(2-(1-(6-(2,4-dimethyl-1H-imidazol-1-yl)pyridin-3-yl)ethylidene)hydrazineyl)quinazoline hydrochloride (Compound 27)
7-chloro-4-(2-(1-(2-(4,5-dimethyl-1H-imidazol-1-yl)pyridin-4-yl)ethylidene)hydrazineyl)quinazoline (Compound 28)
1-(3-(1-(2-(7-chloroquinazolin-4-yl)hydrazineylidene)ethyl)phenyl)—N,N-dimethylmethanamine (Compound 29)
7-chloro-4-(2-(1-(3-(4-methylpiperazin-1-yl)phenyl)ethylidene)hydrazineyl)quinazoline (Compound 30)
6-chloro-N-(6-(4,5-dimethyl-1H-imidazol-1-yl)-5-methoxypyridin-3-yl)phthalazin-1-amine phosphoric acid salt (Compound 31)
(E)-4-(2-(1-(6-(1H-1,2,4-triazol-1-yl)pyridin-3-yl)ethylidene)hydrazineyl)-7-chloroquinazoline (Compound 32)
7-chloro-N-(6-(4,5-dimethyl-1H-imidazol-1-yl)-5-methoxypyridin-3-yl)isoquinolin-4-amine phosphoric acid salt (Compound 33)
N-(6-(1H-1,2,4-triazol-1-yl)pyridin-3-yl)-7-chloroquinazolin-4-amine phosphoric acid salt (Compound 34)
(E)-7-chloro-4-(2-(3-fluoro-4-(1H-imidazol-1-yl)benzylidene)hydrazineyl)quinoline hydrochloride (Compound 35)
4-(2-(1-(4-(1H-imidazol-1-yl)phenyl)ethylidene)hydrazineyl)-7-chloroquinoline phosphate (Compound 36)
7-chloro-N-(6-(2,4-dimethyl-1H-imidazol-1-yl)pyridin-3-yl)isoquinolin-4-amine phosphoric acid salt (Compound 37)
4-(2-(4-(1H-imidazol-1-yl)benzylidene)hydrazineyl)-7-chloroquinoline dihydrochloride (Compound 38)
N-(4-(4-ethylpiperazin-1-yl)phenyl)benzo[g]quinolin-4-amine (Compound 39)
5-((7-chloroquinazolin-4-yl)amino)-2-(4,5-dimethyl-1H-imidazol-1-yl)pyridin-3-ol phosphoric acid salt (Compound 40)
N-(6-(1H-imidazol-1-yl)pyridin-3-yl)-7-chloroquinazolin-4-amine (Compound 41)
7-chloro-N-(3-((diethylamino)methyl)phenyl)quinolin-4-amine (Compound 42)
1-(5-((7-chloroquinazolin-4-yl)amino)pyridin-2-yl)-3-methyl-1H-imidazol-3-ium iodide phosphoric acid salt (Compound 43)
N-(6-(1H-1,2,4-triazol-1-yl)pyridin-3-yl)-7-chloroquinolin-4-amine (Compound 44)
7-chloro-N-(2-((dimethylamino)methyl)pyridin-4-yl)quinolin-4-amine (Compound 45)

As described in Examples, such pharmaceutical compositions contain at least one compound that inhibit interactions between GBAPs and HS-GAGs and therefore, such pharmaceutical compositions may be useful for treatment of amyloid and neurodegenerative diseases. Specifically, as shown in Example 44 and Table 2, compounds of Formula I inhibit binding of Abeta (1-40) and Abeta (1-42) to heparin, a HS-GAG species. Compounds of Formula I inhibit also binding of Abeta (1-42) to human brain membranes purified from human brain, Example 50. As shown in Examples 51-54, Tables 2-6, compounds of Formula I inhibit binding of Abeta to heparin, alpha-synuclein to heparin, binding of tau to heparin, binding of TDP-43 to heparin and binding of SAA to heparin. Compounds of Formula I inhibit also binding of Abeta (1-42) to human brain membranes purified from human brain. Compounds of Formula I inhibit also binding of alpha-synuclein, tau, and TDP-43 to human brain membranes purified from human brain, Examples 51-53.

Inhibition of binding to cell membranes may indicate that the inhibitor compounds are capable of preventing entry of GBAPs into mammalian cells, such as neuronal cells. Consequently, such inhibitor compounds may be useful to prevent propagation of amyloid peptides from cell to cell and spreading amyloidosis.

According to another embodiment, the present disclosure provides a method for the treatment of an amyloid disease, disorder or condition, comprising administering to a subject in need thereof a therapeutic amount of a pharmaceutical composition comprising at least one compound that inhibits binding of a GAG-binding amyloid peptide (GBAP) to a HS-GAG of formula I, wherein the therapeutic amount is effective to inhibit binding of a GBAP to HS-GAG.

According to one embodiment, the present disclosure provides a method of treating a neurodegenerative or amyloid disorder comprising administering to a subject in need thereof a therapeutic amount of a compound that directly binds to a GAG or HS-GAG.

The assays enabling identification of small molecule compounds that bind directly and stably to GAGs, and are capable to inhibit amyloid-beta binding to GAGs are described in Examples.

Also provided are methods of screening compounds to identify inhibitor compounds of GBAP interaction with a GAG such as HS-GAG.

According to one embodiment, this invention provides a method of screening for small organic molecules that directly bind to GAGs and inhibit the binding of GAG-binding amyloid peptides (GBAPs) to GAGs, the method comprising the steps of:
(a) immobilizing a GAG to a surface of a multi-well plate
(b) contacting immobilized GAG with a known quantity of GBAP in the presence of at least one candidate compound; and
(c) measuring the amount of the GBAP bound to the GAG, wherein a significant decrease in GAG-GBAP binding as compared to GAG-GBAP binding in the absence of the candidate compound, identifies said compound as inhibitor of the GAG-GBAP interaction.

According to one embodiment, the GBAP is selected from a group consisting of beta-amyloid, tau, alpha-synuclein, TDP-43, IAPP and SAA. Examples of such assay are given in Examples.

In exemplary embodiments, pharmaceutical compositions and/or formulations as disclosed herein may comprise active agent at a concentration of about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 35%, about 40%, about 45%, about 50%, about 55%, about 60%, about 61%, about 62%, about 63%, about 64%, about 65%, about 66%, about 67%, about 68%, about 69%, about 70%, about 75%, about 75%, and about 80%, In exemplary embodiments, pharmaceutical compositions and/or formulations as disclosed herein may comprise active agent at a concentration of about 1 to about 20%, of about 5% to about 25%, about 10% to about 20%, or about 15% to about 18%, about 30% to about 70%, about 35% to about 65%, about 63.13%, and about 40% to about 64% w/w.

In certain embodiments, the dose of active agent is equal to or greater than, for example, about 0.001, 0.0025 0.005, 0.0075, 0.01, 0.025, 0.05, 0.075, 0.1, 0.25, 0.75, 1, 2, 3, 4, 5, 6, 7, 8, 9, 10, 15, 20, 25, 30, 35, 40, or 45 mg/kg/day. In exemplary embodiments, pharmaceutical compositions and/or formulations of the disclosure may comprise active agent at a concentration of about 0.001, 0.0025 0.005, 0.0075, 0.01, 0.025, 0.05, 0.075, 0.1, 0.25, 0.75, 1, 2, 3, 4, 5, 6, 7, 8, 9, 10, 15, 20, 25, 30, 35, 40, 45, 50, 55, 60, 65, 70, 75, 80, 85, 90, 95, 100, 125, 150, 175, 200, 225, 250, or 275 mg.

In other embodiments, the pharmaceutical compositions as disclosed herein further comprise one or more additional materials such as a pharmaceutically compatible carrier, binder, viscosity modifier, filling agent, suspending agent, flavoring agent, sweetening agent, disintegrating agent, surfactant, preservative, lubricant, colorant, diluent, solubilizer, moistening agent, stabilizer, wetting agent, anti-adherent, parietal cell activator, anti-foaming agent, antioxidant, chelating agent, antifungal agent, antibacterial agent, or one or more combination thereof.

### Screening Assays

The present disclosure provides assays of screening for small molecule compounds in order to identify candidate therapeutics for prevention and/or treatment and/or control of Alzheimer's Disease and other amyloid disorders. The assays provide a means to identify compounds that bind directly to GAGs and prevent amyloid peptide binding to GAGs. To our knowledge, compounds that bind directly and stably to GAGs, and inhibit binding of GAG-binding amyloid peptides (GBAPs) to GAGs, have not been previously described.

According to one embodiment, the present disclosure provides a method of screening for small organic molecules that directly bind to GAGs and inhibit the binding of GAG-binding amyloid peptides (GBAPs) to GAGs, the method comprising the steps of:
(a) Immobilizing a GAG to a surface of a multi-well plate
(b) contacting immobilized GAG with a known quantity of GBAP in the presence of at least one candidate compound; and
(c) measuring the amount of the GBAP bound to the GAG, wherein a significant decrease in GAG-GBAP binding as compared to GAG-GBAP binding in the absence of the candidate compound, identifies said compound as inhibitor of the GAG-GBAP interaction.

According to another embodiment, the present disclosure provides a method of screening for small organic compounds that directly inhibit the interaction of glycosaminoglycans (GAGs) with GAG-binding amyloid peptides (GBAPs), the method comprising the steps of:
(a) Immobilizing a GAG to a surface of a multi-well plate
(b) contacting an immobilized GAG with at least one candidate small organic compound;
(c) removing unbound organic compound;
(d) adding a GBAP; and
(e) measuring the amount of the GBAP bound to the GAG, wherein a significant decrease in GAG-GBAP binding as compared to GAG-GBAP binding in the absence of the candidate compound, identifies said compound as inhibitor of the GAG-GBAP interaction.

According to the present disclosure, small molecule compounds that bind directly and stably to GAGs, and are capable to inhibit a GBAP binding to GAGs can be identified. According to one embodiment, an assay in which the candidate inhibitor compound is first exposed to a GAG in the absence of a GBAP, followed by washing the plate is described. In this manner, only compounds that directly bind to a GAG are identified and the mechanism of action can be defined.

The GAG for assays may be selected from the group consisting of heparan sulfate (HS-GAG), heparin, chondroitin sulfate, dermatan sulfate, keratan sulfate and derivatives and fragments thereof. For instance, chondroitin sulfate may be purified from human brain, conjugated to BSA via its free aldehyde group and the resulting CS-GAG-BSA conjugate immobilized by incubation on a 96-well plate. Some GAGs may be purchased from commercial suppliers. The GAG or a fragment thereof may be purified from an organism, tissue or cell. Alternatively, one may use a proteoglycan as a source of GAG and the proteoglycan may be purified from an organism, tissue, a tumor or a cell by well established methods. Proteoglycan is than used in solution or immobilized on a multi-well plate for compound screening.

According to one embodiment, the GAG is commercially purchased porcine heparin and the heparin is conjugated via its terminal aldehyde to BSA, as described in Example 43. Instead of heparin one may use another GAG. Instead of BSA one may use another carrier protein such as gelatin. The heparin-BSA conjugate is incubated with a multi-well plate (as described in Example 43), which results in immobilization of BSA-heparin to a solid surface of a multi-well plate. Heparin is commonly used instead of HS-GAGs; because of their similarity in chemical structure, data with heparin can be often assumed to apply also to HS-GAGs. Results with heparin-BSA may be confirmed by using HS-GAG-BSA

GBAP is an amyloid peptide or protein, and it can be part of a fusion protein, a protein fragment or a mutant protein. For instance, GBAP may be fused to a tag for subsequent detection of the tag with an antibody. Either GAG or the GBAP may be tagged or labeled. Such label may include, but it is not limited to, a dye, a fluorescent dye, a chemiluminescent agent or a radioactive agent.

According to one embodiment, GBAP is detected by an immunoassay with use of an antibody to GBAP. As described in Experiment 1, an assay for Beta-amyloid was developed by immobilizing heparin-BSA to a plate. After incubation with Beta-amyloid (1-42), the plate was washed and incubated with an antibody to the N-terminus of Beta-amyloid.

According to one embodiment, GBAP binding to GAG is detected by a method selected from, but not limited to, (a) A spectrophotometric detection method; (b) A radioactive detection method; (c) A fluorescent detection method. For example, the antibody to GBAP or a secondary antibody is an antibody conjugated to an enzyme such as horseradish peroxidase (HRP). According to some such embodiments, the enzyme is detected by a colorimetric method with spectrophotometry, as described in Example 43.

According to one embodiment, the GBAP is Beta-amyloid, as described in Examples. The assays may be applicable to other GBAPs as well. GBAP may be in a monomeric or an oligomeric form. Both monomeric and oligomeric forms of Beta-amyloid bind to heparin.

In such assays, typically one is measuring the amount of the GBAP bound to immobilized GAG, wherein a significant decrease in GAG-GBAP binding as compared to GAG-GBAP binding in the absence of the candidate compound, identifies said compound as inhibitor of the GAG-GBAP interaction.

According to one embodiment, the bound GBAP is detected with an anti-GBAP monoclonal antibody (mAb).

Identification of small molecule compounds which interact with heparan sulfate/heparin may be achieved by a variety of techniques. For instance, inhibition of a known protein which binds to HS-GAG may be measured. If a compound inhibits HS-GAG binding to protein X, it either does it by binding to protein X or to HS-GAG. (This can be distinguished for instance, by incubating compound with HS-GAG, a wash step, and subsequent incubation with protein X.) There are many indirect methods for identification/screening for compounds which interact with HS-GAG. A protein X may be labeled with radioactivity, fluorescently or with a tag (which has enzymatic or other activity). If interaction of protein X with HS-GAG is inhibited by compound, then it is achieved either by binding to protein X or to HS-GAG. This invention provides for compounds which bind to HS-GAG and thus prevent binding of protein X.

According to one embodiment, the present disclosure discloses GAGs, specifically HS-GAGs as molecular targets for such screening. The direct targeting of GAGs as described herein is important for efficient drug screening and chemical optimization.

The compound screening methods for identification of inhibitor compounds may be used in various modifications, which are well known to one skilled in the art. Assays can be classified as either direct binding assays or inhibition assays. The GAG molecule may be immobilized, or GBAP may be immobilized or both GAG and GBAP may be present in solution. Detection may focus either on GAG or on GBAP, for instance by using antibodies, biotin-streptavidin, radiolabeling, fluorescent label, etc. Detection methods may also differ, such as spectrophotometry, chemiluminescence, fluorescence, radioactive detection, etc. Immobilized GAGs may be used coated on plates or coupled to beads. GAGs may be linked to a carrier such as a protein, using different chemical methods. Alternatively, the GBAPs may be immobilized, for instance by coating plates or coupling to beads. GBAPs may be used as fusion proteins or domains containing the GAG-binding domain. Another useful approach may be to use as a source of GAG a whole cell, such as an neuronal cell. According to one embodiment, this approach is used to identify Inhibitor Compounds that prevent entry to such nerve cells.

According to one embodiment, compounds for screening may be produced by synthetic chemistry or may be natural compounds, individual or in mixtures, pre-selected by an algorithm, compressed libraries and the like. According to one embodiment, a method of screening is High-Throughput Screening (HTS), in which thousands of compounds are screened with the aid of robotics.

According to one embodiment, compound screening according to the method of the present invention is used as iterative screening in conjunction with chemical optimization via synthetic chemistry.

According to one embodiment, the small organic molecules screened by the methods of the present invention interact with GAGs selected from the group consisting of heparan sulfate (HS-GAG), heparin, chondroitin sulfate, dermatan sulfate, keratan sulfate, and derivatives and fragments thereof.

According to one embodiment, the glycosaminoglycans are HS-GAG or heparin or derivatives and oligosaccharide fragments thereof. GAGs may be crude or purified from an organ, tissue or cell. Such HS-GAGs may be commercially available, or purified from a source of interest such as human liver, human brain, endothelial cells and the like. The HS-GAGs may be also chemically or enzymatically modified, or produced synthetically.

According to one embodiment the small compounds screened by the methods of the present invention interact with proteoglycan containing GAG, for example heparan sulfate proteoglycan (HS-PG). Proteoglycans having HS-GAG chains may be purified from an organ, tissue, cell or tumor. Examples for such HS-PGs are syndecan or agrin. Proteoglycans having other GAG chains, such as versican, may be also used.

Immobilization of a GAG such as heparin can be achieved by a variety of methods. In one embodiment, heparin is bound via its aldehyde group to BSA. Another option is to use Heparin/GAG Binding Plates from Iduron (www.iduron.co.uk), in which case heparin is directly immobilized on a 96-well plate with a modified surface. Among various detection methods, one option is to use a tagged amyloid peptide, or a tagged anti-amyloid peptide antibody. One such tag could consist of biotin, whereas one can use streptavidin (or avidin) conjugated to alkaline phosphatase (or horseradish peroxidase) for the next step. In the case of alkaline phosphatase, the detection is by reaction with p-nitrophenyl phosphate, followed by spectroscopy in an ELISA multi-well plate reader.

When using this kind of assay for compound screening, one may use, for example, a GAG or PG from a target tissue, such as endothelial cell HS-GAG, kidney purified HS-GAG or HS-PG, and the like.

According to one embodiment of the present invention, the inhibitor compounds identified by the methods of the present invention directly interact with GAGs and inhibit their interaction with GBAPs. In principle, the inhibitor compounds can inhibit Beta-amyloid-heparin interaction either (i) by direct binding to heparin and thus preventing its interaction with Beta-amyloid or (ii) by direct binding to Beta-amyloid and subsequently preventing its interaction with heparin.

Compounds found to be suitable for further development and chemical optimization may be further subjected to a second screening, identifying compounds that directly bind to heparin. Individual compounds are incubated with immobilized heparin in the absence of Beta-amyloid. Following washing of the plates to remove all unbound compound, Beta-amyloid is added and the standard assay protocol is followed. As exemplified herein below, compounds found to inhibit heparin-Beta-amyloid binding in the co-incubation assay were found to have same binding capabilities under the pre-incubation conditions. Analysis of IC50's for selected compounds, comparing values in pre-incubation and co-incubation, confirmed the observation. These results show that the compounds inhibit the Beta-amyloid-heparin interaction by direct binding to heparin and not to by binding to Beta-amyloid. Furthermore, the interactions of the compounds with heparin were resistant to washing and therefore relatively stable.

As exemplified for the first time by the present invention structurally diverse compounds are capable of inhibiting GAG interactions with GBAPs.

According to one aspect of the invention, a new molecular target for drug screening, namely a non-protein, non-peptide target known as GAGs, is provided.

Another aspect of this invention provides a mechanism of drug action in the area of amyloidosis, wherein the mechanism comprises direct interaction of small organic molecules with GAGs.

### Assays to Identify Therapeutic Compounds

In their most general form, assays for identifying compounds that act as GAG inhibitors involve contacting the appropriate amyloid peptide, with a putative ligand and measuring its binding properties. Several GAG assays are available to measure the capacity of a compound to bind to GAGs, and such assays are well known in the art. For example, both the GAG and the putative ligand may be in solution for a time sufficient for a complex of the selection and ligand to form, followed by separating the complex from uncomplexed amyloid peptide and ligand, and measuring the amount of complex formed. Alternatively, the amount of uncomplexed amyloid peptide or compound could be measured.

### Treatment Methods

According to one embodiment, a method of treating an amyloid disorder is provided, said method comprising the step of administering to a subject in need thereof a therapeutically effective amount of a compound binding to a GAG and inhibiting GBAP binding to the GAG. GAG may be selected from the group consisting of heparan sulfate (HS-GAG), heparin, chondroitin sulfate, dermatan sulfate, keratan sulfate and a proteoglycan containing GAG.

According to one embodiment, GBAP is Beta-amyloid and the amyloid disorder is Alzheimer's Disease.

According to one embodiment, this invention provides a small molecule compound that:
(i) binds directly to a GAG
   and
(ii) inhibits binding of a GBAP to a GAG
whereas GAG is selected from the group consisting of heparan sulfate (HS-GAG), heparin, chondroitin sulfate, dermatan sulfate, keratan sulfate, and derivatives.

As described in the Background, GAGs appear to have important biological roles in amyloid disorders, for example, in Alzheimer's Disease. Modulating the interactions between GAGs and beta-amyloid peptides therefore may have a significant therapeutic value.

However, with the exception of GAG mimetics, the current art does not provide a method of treatment consisting of inhibiting a GBAP binding to GAGs, for example, HS-GAGs. This invention provides a method of treating an amyloid disorder comprising administering to a subject in need thereof a therapeutic amount of a small molecule compound, the compound being characterized by its ability to directly bind to a GAG, wherein the therapeutic amount is effective to inhibit a GBAP binding to the GAG. According to one embodiment, GBAP is beta-amyloid and the amyloid disorder is Alzheimer's Disease. According to another embodiment, GAG is HS-GAG. According to one embodiment, GBAP is selected from the group alpha-synuclein, tau, TDP-43, SAA and IAPP.

These pharmaceutical compositions may be useful, for example, for the treatment or prevention of amyloid diseases, disorders or conditions. According to one embodiment, the amyloid disease is Alzheimer's Disease or Cerebral Amyloid Angiopathy.

According to one embodiment, the present disclosure provides a method for the treatment of an amyloid disease, disorder or condition associated with inhibition of amyloid beta binding to HS-GAGs, comprises administering to a subject in need thereof a therapeutically effective amount of a pharmaceutical composition comprising at least one compound that inhibits binding of a GAG-binding amyloid peptide (GBAP) to a glycosaminoglycan (GAG) of Formula I.

According to one embodiment, the present disclosure provides a method for the treatment or prevention of an amyloid disease, disorder or condition related to inhibition of amyloid-beta binding to HS-GAGs, comprising administering to a subject in need thereof a therapeutic amount of a pharmaceutical composition comprising at least one compound that inhibits binding of a GAG-binding amyloid peptide (GBAP) to a glycosaminoglycan (GAG) selected from a group consisting of compounds of Formula I, wherein the therapeutic amount is effective to inhibit binding of a GAG-binding amyloid peptide (GBAP) to a glycosaminoglycan (GAG).

According to one embodiment, the present disclosure provides a method of treating an amyloid disorder comprising administering to a subject in need thereof a therapeutic amount of a compound that binds to a glycosaminoglycan (GAG), wherein the therapeutic amount is effective to inhibit the GAG-binding amyloid peptide (GBAP) binding to the GAG.

According to some embodiments, the present disclosure provides a class of compounds that can prevent binding of amyloid-beta to GAGs for the treatment of Alzheimer's Disease (AD). A prominent pathological feature of AD is a robust activation of the neuronal lysosomal pathway, endocytosis and autophagy which are associated with lysosomal cytotoxicity and represent one of the earliest manifestations of sporadic AD (Nixon, R. A., 2008, Autophagy 4(5):590-9). Accumulation, storage and indigestibility of lysosomal HS-GAGs, due to complexation with amyloid peptides, is known to contribute to lysosomal dysfunction and cell death in AD. In addition, HS-GAGs are responsible for internalization and spreading of amyloid proteopathic seeds across the central nervous system (CNS) (Holmes et al. (2013) Proc. Natl. Acad. Sci. U.S.A. 110(33):E3138-47). Without being bound by theory, the compounds of the present disclosure bind to GAGs and prevent amyloid-beta peptide binding to GAGs, thus preventing endocytosis of GAG-amyloid-beta complexes into cells, accumulation in lysosomes, lysosomal dysfunction, cell death and spreading of amyloid disease into unaffected neurons across the CNS.

The present disclosure provides quinoline and quinazoline derivative compounds and pharmaceutical compositions thereof. These compounds inhibit the interactions between GAG-binding amyloid peptides (GBAPs) and heparan sulfate glycosaminoglycans (HS-GAGs) and thus may be useful as therapeutics for the treatment of neurodegenerative diseases associated with amyloidosis and for other amyloid disorders. The present disclosure further provides methods of treatment of neurodegenerative and amyloid diseases.

TABLE 1
List of Compounds with Structures

| Com- | |
|---|---|
| pound | |
| No. | Structure |
| 1 | 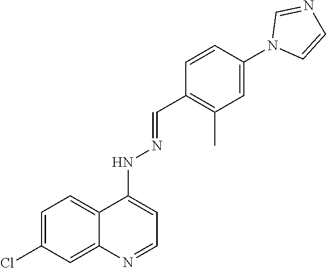 [SMILES: Cc1cc(-n2ccnc2)ccc1/C=N/Nc1ccnc2cc(Cl)ccc12] |
| | Chemical Formula: C₂₀H₁₆ClN₅ |
| | Molecular Weight: 361.8330 |
| 2 | 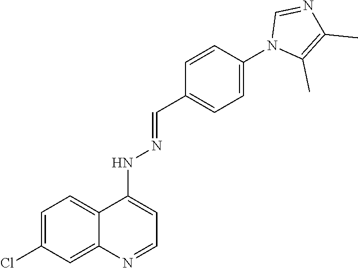 [SMILES: Cc1ncn(-c2ccc(/C=N/Nc3ccnc4cc(Cl)ccc34)cc2)c1C] |
| | Chemical Formula: C₂₁H₁₈ClN₅ |
| | Molecular Weight: 375.8600 |
| 3 | 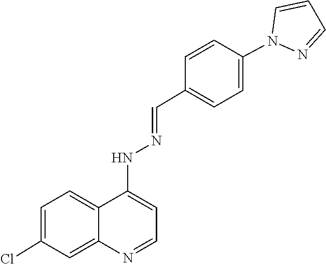 [SMILES: Clc1ccc2c(N/N=C/c3ccc(-n4cccn4)cc3)ccnc2c1] |
| | Chemical Formula: C₁₉H₁₄ClN₅ |
| | Molecular Weight: 347.8060 |
| 4 | 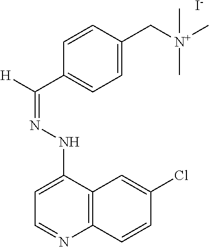 [SMILES: [I-] ; C[N+](C)(C)Cc1ccc(/C=N\Nc2ccnc3ccc(Cl)cc23)cc1] |
| | Chemical Formula: C₂₀H₂₂ClN₄⁺ |
| | Molecular Weight: 353.87 |
| 5 | 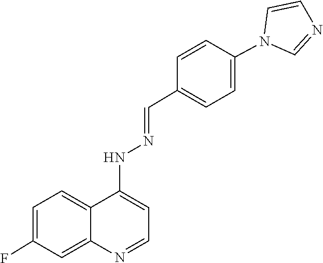 [SMILES: Fc1ccc2c(N/N=C/c3ccc(-n4ccnc4)cc3)ccnc2c1] |
| | Chemical Formula: C₁₉H₁₄FN₅ |
| | Molecular Weight: 331.3544 |
| 6 | 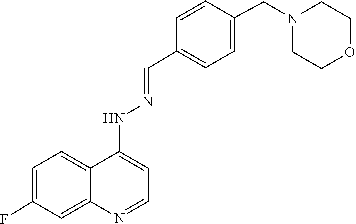 [SMILES: Fc1ccc2c(N/N=C/c3ccc(CN4CCOCC4)cc3)ccnc2c1] |
| | Chemical Formula: C₂₁H₂₁FN₄O |
| | Molecular Weight: 364.4244 |
| 7 | 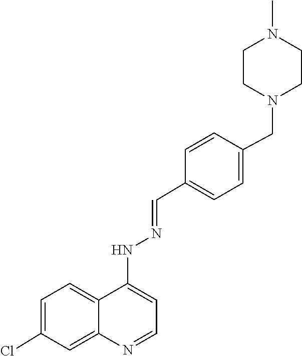 [SMILES: CN1CCN(Cc2ccc(/C=N/Nc3ccnc4cc(Cl)ccc34)cc2)CC1] |
| | Chemical Formula: C₂₂H₂₄ClN₅ |
| | Molecular Weight: 393.9190 |
| 8 | 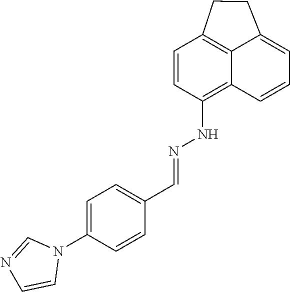 [SMILES: C(=N\Nc1ccc2c3c(cccc13)CC2)/c1ccc(-n2ccnc2)cc1] |
| | Chemical Formula: C₂₂H₁₈N₄ |
| | Molecular Weight: 338.4140 |
| 9 | 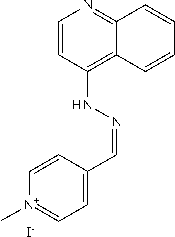 [SMILES: C[n+]1ccc(/C=N\Nc2ccnc3ccccc23)cc1 ; [I-]] |
| | Chemical Formula: C₁₆H₁₅IN₄ |
| | Molecular Weight: 390.2285 |
| 10 | 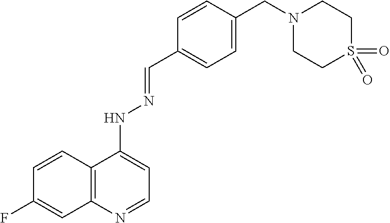 [SMILES: O=S1(=O)CCN(Cc2ccc(/C=N/Nc3ccnc4cc(F)ccc34)cc2)CC1] |
| | Chemical Formula: C₂₁H₂₁FN₄O₂S |
| | Molecular Weight: 412.4834 |
| 11 | 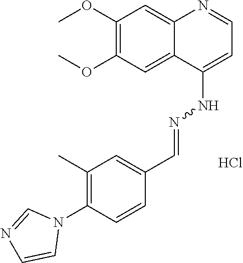 [SMILES: COc1cc2nccc(NN=Cc3ccc(-n4ccnc4)c(C)c3)c2cc1OC ; Cl] |
| | Chemical Formula: C₂₂H₂₂ClN₅O₂ |
| | Molecular Weight: 423.9010 |
| 12 | 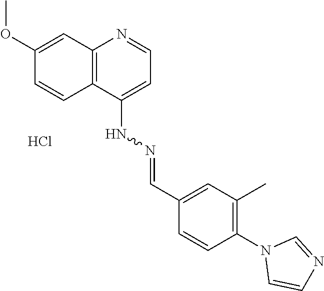 [SMILES: COc1ccc2c(NN=Cc3ccc(-n4ccnc4)c(C)c3)ccnc2c1 ; Cl] |
| | Chemical Formula: C₂₁H₂₀ClN₅O |
| | Molecular Weight: 393.8750 |
| 13 | 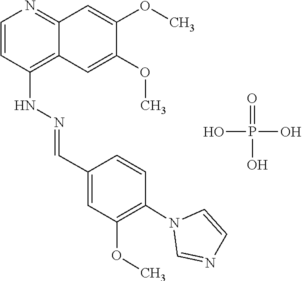 [SMILES: COc1cc2nccc(N/N=C/c3ccc(-n4ccnc4)c(OC)c3)c2cc1OC ; O=P(O)(O)O] |
| | Chemical Formula: C₂₂H₂₁N₅O₃ |
| | Molecular Weight: 403.44 |
| 14 | 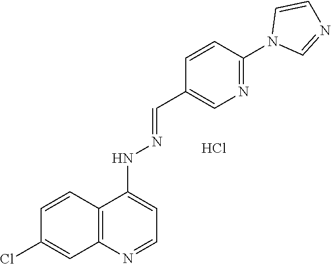 [SMILES: Clc1ccc2c(N/N=C/c3ccc(-n4ccnc4)nc3)ccnc2c1 ; Cl] |
| | Chemical Formula: C₁₈H₁₃ClN₆ |
| | Molecular Weight: 348.79 |
| 15 | 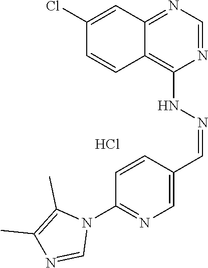 [SMILES: Cc1ncn(-c2ccc(/C=N\Nc3ncnc4cc(Cl)ccc34)cn2)c1C ; Cl] |
| | Chemical Formula: C₁₉H₁₇Cl₂N₇ |
| | Molecular Weight: 414.2940 |
| 16 | 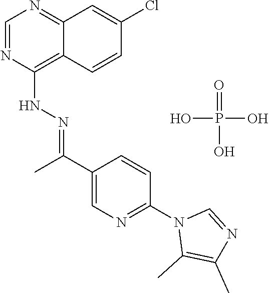 [SMILES: C/C(=N\Nc1ncnc2cc(Cl)ccc12)c1ccc(-n2cnc(C)c2C)nc1 ; O=P(O)(O)O] |
| | Chemical Formula: C₂₀H₁₈ClN₇ |
| | Molecular Weight: 391.86 |
| 17 | 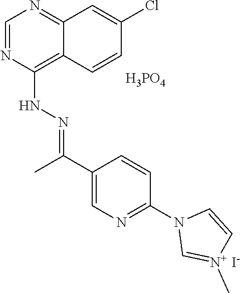 |
| | Chemical Formula: C₁₉H₁₇ClN₇⁺ |
| | Molecular Weight: 378.84 |
| 18 | 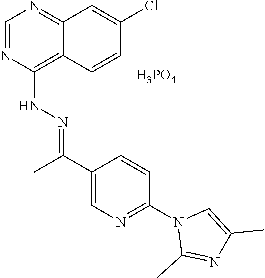 |
| | Chemical Formula: C₂₀H₁₈ClN₇ |
| | Molecular Weight: 391.86 |
| 19 | 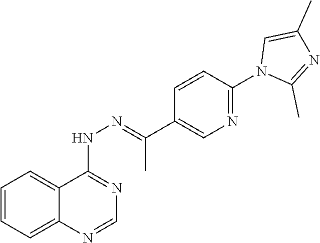 [SMILES: C/C(=N\Nc1ncnc2ccccc12)c1ccc(-n2cc(C)nc2C)nc1] |
| | Chemical Formula: C₂₀H₁₉N₇ |
| | Molecular Weight: 357.4210 |
| 20 | 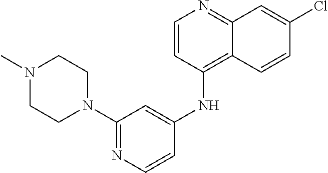 [SMILES: CN1CCN(c2cc(Nc3ccnc4cc(Cl)ccc34)ccn2)CC1] |
| | Chemical Formula: C₁₉H₂₀ClN₅ |
| | Molecular Weight: 353.85 |
| 21 | 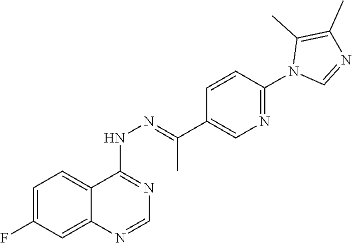 [SMILES: C/C(=N\Nc1ncnc2cc(F)ccc12)c1ccc(-n2cnc(C)c2C)nc1] |
| | Chemical Formula: C₂₀H₁₈FN₇ |
| | Molecular Weight: 375.41 |
| 22 | 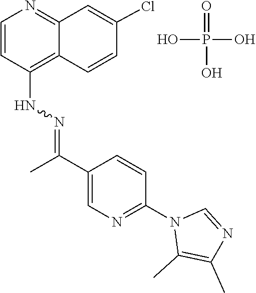 |
| | Chemical Formula: C₂₁H₁₉ClN₆ |
| | Molecular Weight: 390.88 |
| 23 | 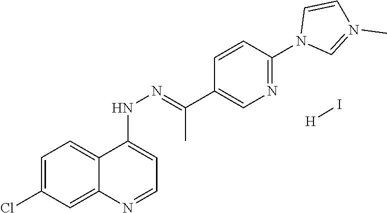 |
| | Chemical Formula: C₂₀H₁₉ClIN₆ |
| | Molecular Weight: 505.7685 |
| 24 | 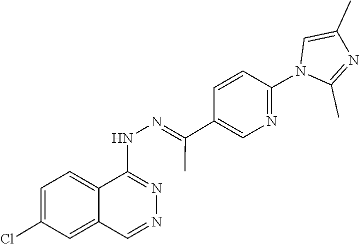 [SMILES: C/C(=N\Nc1nncc2cc(Cl)ccc12)c1ccc(-n2cc(C)nc2C)nc1] |
| | Chemical Formula: C₂₀H₁₈ClN₇ |
| | Molecular Weight: 391.8630 |
| 25 | 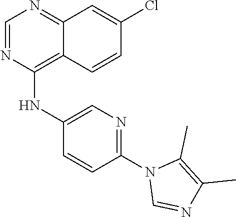 [SMILES: Cc1ncn(-c2ccc(Nc3ncnc4cc(Cl)ccc34)cn2)c1C] |
| | Chemical Formula: C₁₈H₁₅ClN₆ |
| | Molecular Weight: 350.81 |
| 26 | 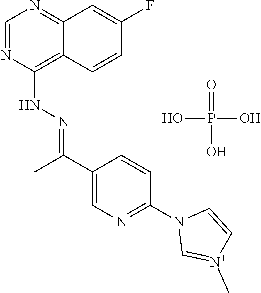 [SMILES: C/C(=N\Nc1ncnc2cc(F)ccc12)c1ccc(-n2cc[n+](C)c2)nc1 ; O=P(O)(O)O] |
| | Chemical Formula: C₁₉H₁₇FN₇⁺ |
| | Molecular Weight: 362.39 |
| 27 | 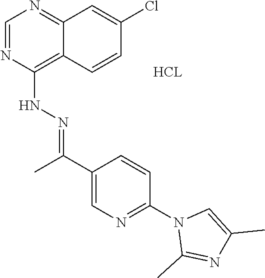 [SMILES: C/C(=N\Nc1ncnc2cc(Cl)ccc12)c1ccc(-n2cc(C)nc2C)nc1] |
| | Chemical Formula: C₂₀H₁₈ClN₇ |
| | Molecular Weight: 391.86 |
| 28 | 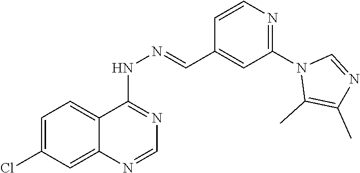 [SMILES: Cc1ncn(-c2cc(/C=N/Nc3ncnc4cc(Cl)ccc34)ccn2)c1C] |
| | Chemical Formula: C₁₉H₁₆ClN₇ |
| | Molecular Weight: 377.8360 |
| 29 | 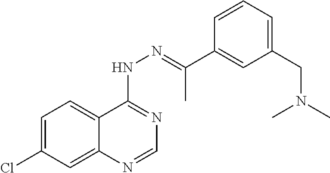 [SMILES: C/C(=N\Nc1ncnc2cc(Cl)ccc12)c1cccc(CN(C)C)c1] |
| | Chemical Formula: C₁₉H₂₀ClN₅ |
| | Molecular Weight: 353.8540 |
| 30 | 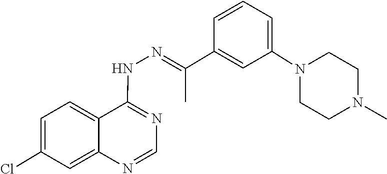 [SMILES: C/C(=N\Nc1ncnc2cc(Cl)ccc12)c1cccc(N2CCN(C)CC2)c1] |
| | Chemical Formula: C₂₁H₂₃ClN₆ |
| | Molecular Weight: 394.9070 |
| 31 | 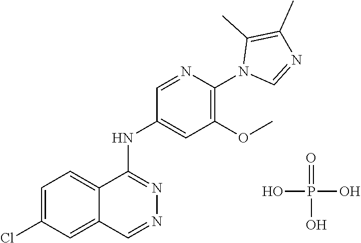 [SMILES: COc1cc(Nc2nncc3cc(Cl)ccc23)cnc1-n1cnc(C)c1C ; O=P(O)(O)O] |
| | Chemical Formula: C₁₉H₁₇ClN₆O |
| | Molecular Weight: 380.8360 |
| 32 | 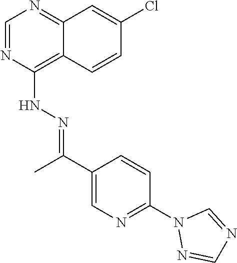 [SMILES: C/C(=N\Nc1ncnc2cc(Cl)ccc12)c1ccc(-n2cncn2)nc1] |
| | Chemical Formula: C₁₇H₁₃ClN₈ |
| | Molecular Weight: 364.80 |
| 33 | 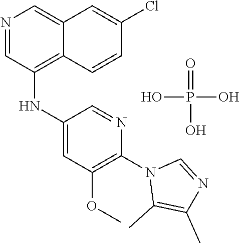 |
| | Chemical Formula: C₂₀H₁₈ClN₅O |
| | Molecular Weight: 379.85 |
| 34 | 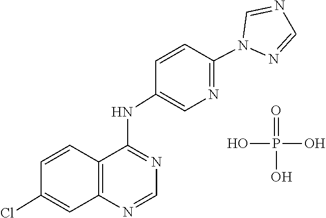 [SMILES: Clc1ccc2c(Nc3ccc(-n4cncn4)nc3)ncnc2c1 ; O=P(O)(O)O] |
| | Chemical Formula: C₁₅H₁₀ClN₇ |
| | Molecular Weight: 323.74 |
| 35 | 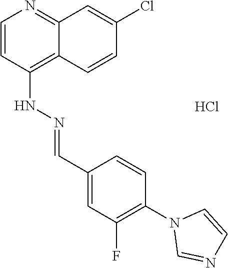 [SMILES: Fc1cc(/C=N/Nc2ccnc3cc(Cl)ccc23)ccc1-n1ccnc1 ; Cl] |
| | Chemical Formula: C₁₉H₁₃ClFN₅ |
| | Molecular Weight: 365.80 |
| 36 | 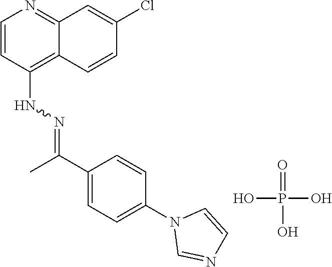 |
| | Chemical Formula: C₂₀H₁₉ClN₅O₄P |
| | Molecular Weight: 459.8268 |
| 37 | 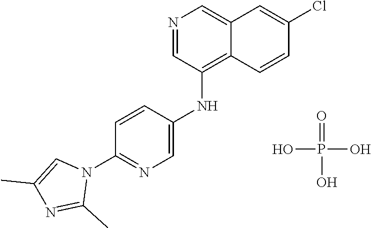 [SMILES: Cc1cn(-c2ccc(Nc3cncc4cc(Cl)ccc34)cn2)c(C)n1 ; O=P(O)(O)O] |
| | Chemical Formula: C₁₉H₁₆ClN₅ |
| | Molecular Weight: 349.82 |
| 38 | 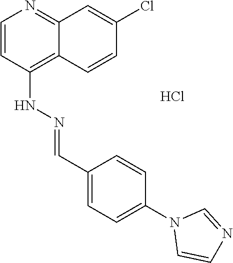 [SMILES: Clc1ccc2c(N/N=C/c3ccc(-n4ccnc4)cc3)ccnc2c1 ; Cl] |
| | Chemical Formula: C₁₉H₁₄ClN₅ |
| | Molecular Weight: 347.81 |
| 39 | 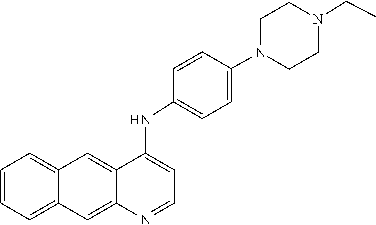 [SMILES: CCN1CCN(c2ccc(Nc3ccnc4cc5ccccc5cc34)cc2)CC1] |
| | Chemical Formula: C₂₅H₂₆N₄ |
| | Molecular Weight: 382.51 |
| 40 | 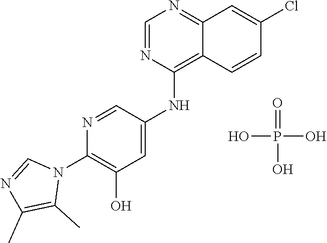 [SMILES: Cc1ncn(-c2ncc(Nc3ncnc4cc(Cl)ccc34)cc2O)c1C ; O=P(O)(O)O] |
| | Chemical Formula: C₁₈H₁₅ClN₆O |
| | Molecular Weight: 366.81 |
| 41 | 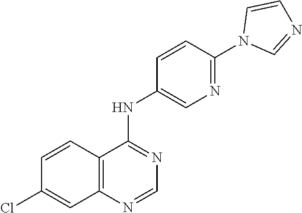 [SMILES: Clc1ccc2c(Nc3ccc(-n4ccnc4)nc3)ncnc2c1] |
| | Chemical Formula: C₁₆H₁₁ClN₆ |
| | Molecular Weight: 322.76 |
| 42 | 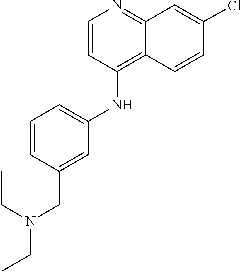 [SMILES: CCN(CC)Cc1cccc(Nc2ccnc3cc(Cl)ccc23)c1] |
| | Chemical Formula: C₂₀H₂₂ClN₃ |
| | Molecular Weight: 339.87 |
| 43 | 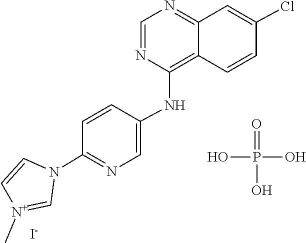 [SMILES: C[n+]1ccn(-c2ccc(Nc3ncnc4cc(Cl)ccc34)cn2)c1 ; [I-] ; O=P(O)(O)O] |
| | Chemical Formula: C₁₇H₁₄ClIN₆ |
| | Molecular Weight: 464.70 |
| 44 | 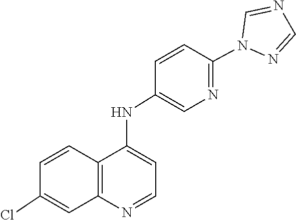 [SMILES: Clc1ccc2c(Nc3ccc(-n4cncn4)nc3)ccnc2c1] |
| | Chemical Formula: C₁₆H₁₁ClN₆ |
| | Molecular Weight: 322.76 |
| 45 | 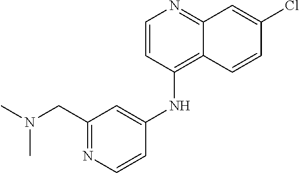 [SMILES: CN(C)Cc1cc(Nc2ccnc3cc(Cl)ccc23)ccn1] |
| | Chemical Formula: C₁₇H₁₇ClN₄ |
| | Molecular Weight: 312.80 |

### List of Compounds with Names

(E)-4-((E)-(4-(1H-imidazol-1-yl)-2-methylbenzylidene)hydrazono)-7-chloro-1,4-dihydroquinoline (Compound 1)
(E)-7-chloro-4-((E)-(4-(4,5-dimethyl-1H-imidazol-1-yl)benzylidene)hydrazono)-1,4-dihydroquinoline (Compound 2)
(E)-4-((E)-(4-(1H-pyrazol-1-yl)benzylidene)hydrazono)-7-chloro-1,4-dihydroquinoline (Compound 3)
(Z)-1-(4-((2-(6-chloroquinolin-4-yl)hydrazineylidene)methyl)phenyl)—N,N,N-trimethylmethanaminium iodide (Compound 4)
(E)-4-(2-(4-(1H-imidazol-1-yl)benzylidene)hydrazinyl)-7-fluoroquinoline (Compound 5)
(E)-4-(4-((2-(7-fluoroquinolin-4-yl)hydrazono)methyl)benzyl)morpholine (Compound 6)
(E)-7-chloro-4-(2-(4-((4-methylpiperazin-1-yl)methyl)benzylidene)hydrazinyl)quinolone (Compound 7)
(E)-1-(4-((2-(1,2-dihydroacenaphthylen-5-yl)hydrazineylidene)methyl)phenyl)-1H-imidazole (Compound 8)
(Z)-1-methyl-4-((2-(quinolin-4-yl)hydrazono)methyl)pyridin-1-ium iodide (Compound 9)
(E)-4-(4-((2-(7-fluoroquinolin-4-yl)hydrazono)methyl)benzyl)thiomorpholine 1,1-dioxide (Compound 10)
4-(2-(4-(1H-imidazol-1-yl)-3-methylbenzylidene)hydrazineyl)-6,7-dimethoxyquinoline hydrochloride (Compound 11)
4-(2-(4-(1H-imidazol-1-yl)-3-methylbenzylidene)hydrazineyl)-7-methoxyquinoline (Compound 12)
(E)-4-(2-(4-(1H-imidazol-1-yl)-3-methoxybenzylidene)hydrazineyl)-6,7-dimethoxyquinoline phosphate (Compound 13)
(E)-4-(2-((6-(1H-imidazol-1-yl)pyridin-3-yl)methylene)hydrazineyl)-7-chloroquinoline hydrochloride (Compound 14)
7-chloro-4-(2-((6-(4,5-dimethyl-1H-imidazol-1-yl)pyridin-3-yl)methylene)hydrazineyl)quinazoline hydrochloride (Compound 15)
7-chloro-4-(2-(1-(6-(4,5-dimethyl-1H-imidazol-1-yl)pyridin-3-yl)ethylidene)hydrazineyl)quinazoline phosphate (Compound 16)
(E)-1-(5-(1-(2-(7-chloroquinazolin-4-yl)hydrazineylidene)ethyl)pyridin-2-yl)-3-methyl-1H-imidazol-3-ium iodide phosphoric acid salt (Compound 17)
(E)-7-chloro-4-(2-(1-(6-(2,4-dimethyl-1H-imidazol-1-yl)pyridin-3-yl)ethylidene)hydrazineyl)quinazoline phosphoric acid salt (Compound 18)
(E)-4-(2-(1-(6-(2,4-dimethyl-1H-imidazol-1-yl)pyridin-3-yl)ethylidene)hydrazineyl)quinazoline (Compound 19)
7-chloro-N-(2-(4-methylpiperazin-1-yl)pyridin-4-yl)quinolin-4-amine (Compound 20)
(E)-4-(2-(1-(6-(4,5-dimethyl-1H-imidazol-1-yl)pyridin-3-yl)ethylidene)hydrazineyl)-7-fluoroquinazoline (Compound 21)
7-chloro-4-(2-(1-(6-(4,5-dimethyl-1H-imidazol-1-yl)pyridin-3-yl)ethylidene)hydrazineyl)quinoline (Compound 22)
1-(5-(1-(2-(7-chloroquinolin-4-yl)hydrazineylidene)ethyl)pyridin-2-yl)-3-methyl-1H-imidazol-3-ium iodide (Compound 23)
(E)-6-chloro-1-(2-(1-(6-(2,4-dimethyl-1H-imidazol-1-yl)pyridin-3-yl)ethylidene)hydrazineyl)phthalazine (Compound 24)
7-chloro-N-(6-(4,5-dimethyl-1H-imidazol-1-yl)pyridin-3-yl)quinazolin-4-amine (Compound 25)
(E)-1-(5-(1-(2-(7-fluoroquinazolin-4-yl)hydrazineylidene)ethyl)pyridin-2-yl)-3-methyl-1H-imidazol-3-ium phosphoric acid salt (Compound 26)
(E)-7-chloro-4-(2-(1-(6-(2,4-dimethyl-1H-imidazol-1-yl)pyridin-3-yl)ethylidene)hydrazineyl)quinazoline hydrochloride (Compound 27)
7-chloro-4-(2-(1-(2-(4,5-dimethyl-1H-imidazol-1-yl)pyridin-4-yl)ethylidene)hydrazineyl)quinazoline (Compound 28)
1-(3-(1-(2-(7-chloroquinazolin-4-yl)hydrazineylidene)ethyl)phenyl)—N,N-dimethylmethanamine (Compound 29)
7-chloro-4-(2-(1-(3-(4-methylpiperazin-1-yl)phenyl)ethylidene)hydrazineyl)quinazoline (Compound 30)
6-chloro-N-(6-(4,5-dimethyl-1H-imidazol-1-yl)-5-methoxypyridin-3-yl)phthalazin-1-amine phosphoric acid salt (Compound 31)
(E)-4-(2-(1-(6-(1H-1,2,4-triazol-1-yl)pyridin-3-yl)ethylidene)hydrazineyl)-7-chloroquinazoline (Compound 32)
7-chloro-N-(6-(4,5-dimethyl-1H-imidazol-1-yl)-5-methoxypyridin-3-yl)isoquinolin-4-amine phosphoric acid salt (Compound 33)
N-(6-(1H-1,2,4-triazol-1-yl)pyridin-3-yl)-7-chloroquinazolin-4-amine phosphoric acid salt (Compound 34)
(E)-7-chloro-4-(2-(3-fluoro-4-(1H-imidazol-1-yl)benzylidene)hydrazineyl)quinoline hydrochloride (Compound 35)
4-(2-(1-(4-(1H-imidazol-1-yl)phenyl)ethylidene)hydrazineyl)-7-chloroquinoline phosphate (Compound 36)
7-chloro-N-(6-(2,4-dimethyl-1H-imidazol-1-yl)pyridin-3-yl)isoquinolin-4-amine phosphoric acid salt (Compound 37)
4-(2-(4-(1H-imidazol-1-yl)benzylidene)hydrazineyl)-7-chloroquinoline dihydrochloride (Compound 38)
N-(4-(4-ethylpiperazin-1-yl)phenyl)benzo[g]quinolin-4-amine (Compound 39)
5-((7-chloroquinazolin-4-yl)amino)-2-(4,5-dimethyl-1H-imidazol-1-yl)pyridin-3-ol phosphoric acid salt (Compound 40)
N-(6-(1H-imidazol-1-yl)pyridin-3-yl)-7-chloroquinazolin-4-amine (Compound 41)
7-chloro-N-(3-((diethylamino)methyl)phenyl)quinolin-4-amine (Compound 42)
1-(5-((7-chloroquinazolin-4-yl)amino)pyridin-2-yl)-3-methyl-1H-imidazol-3-ium iodide phosphoric acid salt (Compound 43)
N-(6-(1H-1,2,4-triazol-1-yl)pyridin-3-yl)-7-chloroquinolin-4-amine (Compound 44) 7-chloro-N-(2-((dimethylamino)methyl)pyridin-4-yl)quinolin-4-amine (Compound 45)

### Dosage Forms

The pharmaceutical compositions as disclosed herein can provided in the form of a minicapsule, a capsule, a tablet, an implant, a troche, a lozenge (minitablet), a temporary or permanent suspension, an ovule, a suppository, a wafer, a chewable tablet, a quick or fast dissolving tablet, an effervescent tablet, a granule, a film, a sprinkle, a pellet, a bead, a pill, a powder, a triturate, a platelet, a strip or a sachet. Compositions can also be administered after being mixed with, for example yoghurt or fruit juice and swallowed or followed with a drink or beverage. These forms are well known in the art and are packaged appropriately. The compositions can be formulated for oral or rectal delivery.

Tablets prepared for oral administration according to the invention, and manufactured using direct compression, will generally contain other inactive additives such as binders, lubricants, disintegrants, fillers, stabilizers, surfactants, coloring agents, and the like. Binders are used to impart cohesive qualities to a tablet, and thus ensure that the tablet remains intact after compression. Suitable binder materials include, but are not limited to, starch (including corn starch and pregelatinized starch), gelatin, sugars (including sucrose, glucose, dextrose and lactose), polyethylene glycol, waxes, and natural and synthetic gums, e.g., acacia sodium alginate, polyvinylpyrrolidone, cellulosic polymers (including hydroxypropyl cellulose, hydroxypropyl methylcellulose, methyl cellulose, microcrystalline cellulose, ethyl cellulose, hydroxyethyl cellulose, and the like), and Veegum. Lubricants are used to facilitate tablet manufacture, promoting powder flow and preventing particle capping (i.e., particle breakage) when pressure is relieved. Useful lubricants are magnesium stearate ( ), calcium stearate, stearic acid, and hydrogenated vegetable oil (preferably comprised of hydrogenated and refined triglycerides of stearic and palmitic acids at about 1 wt. % to 5 wt. %, most preferably less than about 2 wt. %). Lubricants may be present in a concentration of, for example, from about 0.25 wt. % to about 3 wt. %, 0.5 wt. % to about 2.0 wt. %, from about 0.75% to about 1.5%.

Disintegrants are used to facilitate disintegration of the tablet, thereby increasing the erosion rate relative to the dissolution rate, and are generally starches, clays, celluloses, algins, gums, or crosslinked polymers (e.g., crosslinked polyvinyl pyrrolidone). Fillers include, for example, materials such as silicon dioxide, titanium dioxide, alumina, talc, kaolin, powdered cellulose, and microcrystalline cellulose, as well as soluble materials such as mannitol, urea, sucrose, lactose, lactose monohydrate, dextrose, sodium chloride, and sorbitol. Solubility-enhancers, including solubilizers per se, emulsifiers, and complexing agents (e.g., cyclodextrins), may also be advantageously included in the present formulations. Stabilizers, as well known in the art, are used to inhibit or retard drug decomposition reactions that include, by way of example, oxidative reactions. Disintegrants may be present in a concentration of, for example, from about 0.25 wt. % to about 3 wt. %, 0.5 wt. % to about 2.0 wt. %, from about 0.75% to about 1.5%.

Shellac, also called purified lac, a refined product obtained from the, resinous secretion of an insect. This coating dissolves in media of pH>7.

Colorants, detackifiers, surfactants, antifoaming agents, lubricants, stabilizers such as hydroxy propyl cellulose, acid/base may be added to the coatings besides plasticizers to solubilize or disperse the coating material, and to improve coating performance and the coated product.

In carrying out the method as disclosed herein, the combination of the invention may be administered to mammalian species, such as dogs, cats, humans, etc. and as such may be incorporated in a conventional systemic dosage form, such as a tablet, capsule, or elixir. The above dosage forms will also include the necessary carrier material, excipient, viscosity modifier, lubricant, buffer, antibacterial, bulking agent (such as mannitol), anti-oxidants (ascorbic acid of sodium bisulfate) or the like.

The dose administered may be carefully adjusted according to age, weight and condition of the patient or subject, as well as the route of administration, dosage form and regimen and the desired result.

The compositions of the invention may be administered in the dosage forms in single or divided doses of one to four times daily, or may be administered multiple times per day. It may be advisable to start a patient on a low dose combination and work up gradually to a high dose combination.

Tablets of various sizes can be prepared, e.g., of about 2 to 2000 mg in total weight, containing one or both of the active ingredients, with the remainder being a physiologically acceptable carrier of other materials according to accepted practice. Gelatin capsules can be similarly formulated.

Liquid formulations can also be prepared by dissolving or suspending one or the combination of active substances in a conventional liquid vehicle acceptable for administration so as to provide the desired dosage in, for example, one to four teaspoonfuls.

Dosage forms can be administered to the patient on a regimen of, for example, one, two, three, four, five, six, or other multiple doses per day.

In order to more finely regulate the dosage schedule, the active substances may be administered separately in individual dosage units at the same time or carefully coordinated times. The respective substances can be individually formulated in separate unit dosage forms in a manner similar to that described above.

In formulating the compositions, the active substances, in the amounts described above, may be compounded according to accepted practice with a physiologically acceptable vehicle, carrier, excipient, binder, viscosity modifier, preservative, stabilizer, flavor, etc., in the particular type of unit dosage form.

The invention will be illustrated in more detail with reference to the following Examples, but it should be understood that the present invention is not deemed to be limited thereto.

### EXAMPLES

### Synthetic Chemistry Experimental Procedures

### General Methods

### HPLC Purification, Method A:

Purification was performed using IPLC (H₂O—MeOH; Agilent 1260 Infinity systems equipped with DAD and mass-detectors. Waters Sunfire C18 OBD Prep Column, 100 Å, 5 μm, 19 mm×100 mm with SunFire C18 Prep Guard Cartridge, 100 Å, 10 μm, 19 mm×10 mm) The material was dissolved in 0.7 mL DMSO. Flow: 30 mL/min. Purity of the obtained fractions was checked via the analytical LCMS. Spectra were recorded for each fraction as it was obtained straight after chromatography in the solution form. The solvent was evaporated in the flow of N2 at 80° C. Appropriate fractions were combined after dissolution in 0.5 mL MeOH followed by solvent removal under a flow of N2 at 80° C.

### NMR

Bruker AVANCE DRX 500 or Varian UNITYplus 400

### Analytical LC/MS, Method 1

Agilent 1100 Series LC/MSD system with DAD\ELSD and Agilent LC\MSD VL (G1956 Å), SL (G1956B) mass-spectrometer.

Agilent 1200 Series LC/MSD system with DAD\ELSD and Agilent LC\MSD SL (G6130 Å), SL (G6140 Å) mass-spectrometer.

All the LC/MS data were obtained using positive/negative mode switching.
Column Zorbax SB-C18 1.8 μm 4.6×15 mm Rapid Resolution cartridge (PN 821975-932)
Mobile phase A—acetonitrile, 0.1% formic acid
   B—water (0.1% formic acid)
Flow rate 3 ml/min
Gradient 0 min*100% B
0.01 min-100% B
1.5 min-0% B
1.8 min-0% B
1.81 min-100% B
Injection volume 1 μl
Ionization mode atmospheric pressure chemical ionization (APCI)
Scan range m/z 80-1000.

### LIST OF ABBREVIATIONS

THF tetrahydrofuran
DMF N,N-dimethylformamide
MeOH methanol
iPrOH isopropyl alcohol
DCM dichloromethane
MeCN or ACN acetonitrile
PE petroleum ether
EtOAc ethyl acetate
Et₃N or TEA, triethylamine
DMSO dimethyl sulfoxide
LC liquid chromatography
HPLC high-performance liquid chromatography
MS mass spectrometry
LCMS liquid chromatography-mass spectrometry
NMR nuclear magnetic resonance
HOAc acetic acid

### Example 1: Preparation of (E)-4-((E)-(4-(1H-imidazol-1-yl)-2-methylbenzylidene)hydrazono)-7-chloro-1,4-dihydroquinoline (1)

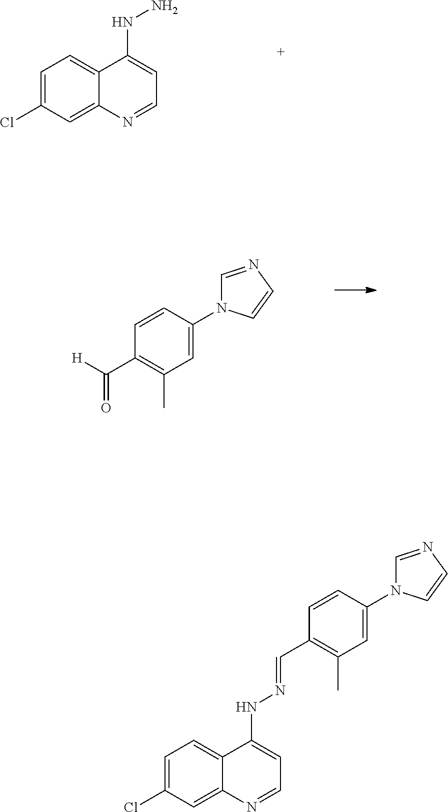 [SMILES: NNc1ccnc2cc(Cl)ccc12 ; Cc1cc(-n2ccnc2)ccc1C=O ; Cc1cc(-n2ccnc2)ccc1/C=N/Nc1ccnc2cc(Cl)ccc12]

Same conditions as compound 10 using 7-chloro-4-hydrazinylquinoline and 4-(1H-imidazol-1-yl)-2-methylbenzaldehyde. Yield 41%.

¹H NMR (400 MHz, DMSO-d₆) δ 11.46 (s, NH), 8.68-7.13 (m, Ar, 12H), 2.58 (s, 3H).

LCMS Rₜ=0.88 min using Method 1, MS ES-API calcd. for C₂₀H₁₆ClN₅ [M+H]⁺ 362.8, found 361.1.

### Example 2: Preparation of (E)-7-chloro-4-((E)-(4-(4,5-dimethyl-1H-imidazol-1-yl)benzylidene)hydrazono)-1,4-dihydroquinoline (2)

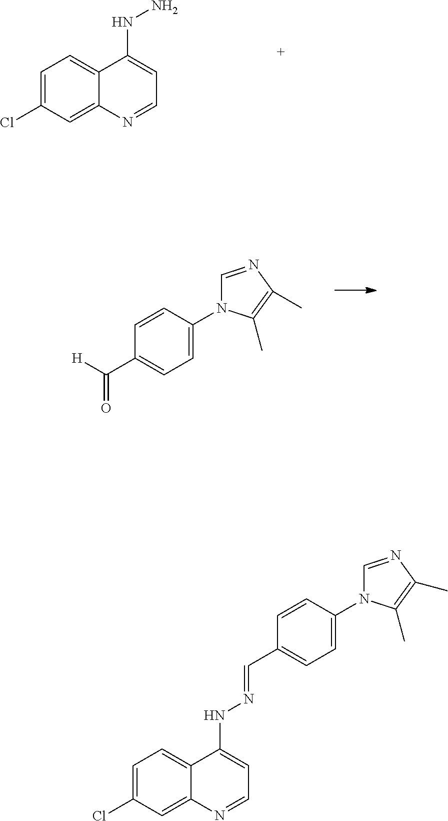 [SMILES: NNc1ccnc2cc(Cl)ccc12 ; Cc1ncn(-c2ccc(C=O)cc2)c1C ; Cc1ncn(-c2ccc(/C=N/Nc3ccnc4cc(Cl)ccc34)cc2)c1C]

Same conditions as compound 10 using 7-chloro-4-hydrazinylquinoline and 4-(4,5-dimethyl-1H-imidazol-1-yl)benzaldehyde. Yield 37%.

¹H NMR (400 MHz, DMSO-d₆) δ 11.34 (s, H), 8.46-7.26 (m, 10H, Ar), 2.12 (s, 6H).

LCMS Rₜ=0.88 min using Method 1, MS ES-API calcd. for C₂₁H₁₈ClN₅ [M+H]⁺ 376.9, found 375.2.

### Example 3: Preparation of (E)-4-((E)-(4-(1H-pyrazol-1-yl)benzylidene)hydrazono)-7-chloro-1,4-dihydroquinoline (3)

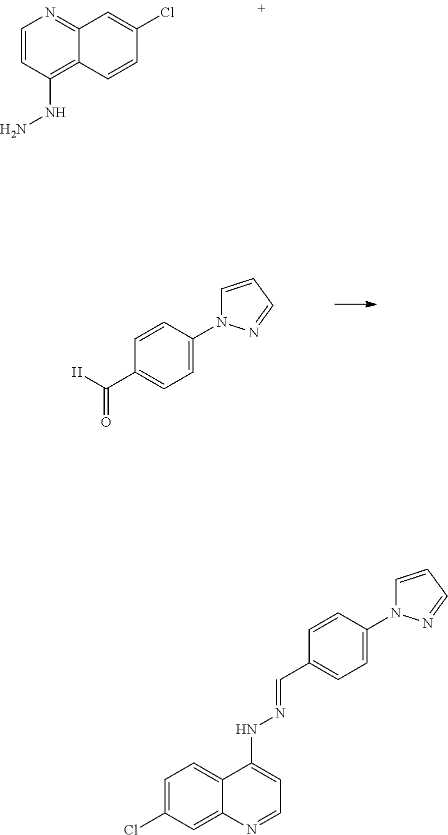 [SMILES: NNc1ccnc2cc(Cl)ccc12 ; O=Cc1ccc(-n2cccn2)cc1 ; Clc1ccc2c(N/N=C/c3ccc(-n4cccn4)cc3)ccnc2c1]

Same conditions as compound 10 using 7-chloro-4-hydrazinylquinoline and 4-(1H-pyrazol-1-yl)benzaldehyde, Yield: 35%.

¹H NMR (400 MHz, DMSO-d₆) δ 11.38 (s, NH), 8.45-6.52 (m, 13H, Ar)

LCMS Rₜ=1.1 min using Method 1, MS ES calcd. for C₁₉H₁₄ClN₅ [M+H]⁺ 348.1, found 347.1.

### Example 4: (Z)-1-(4-((2-(6-chloroquinolin-4-yl)hydrazineylidene)methyl)phenyl)—N,N,N-trimethylmethanaminium iodide (4)

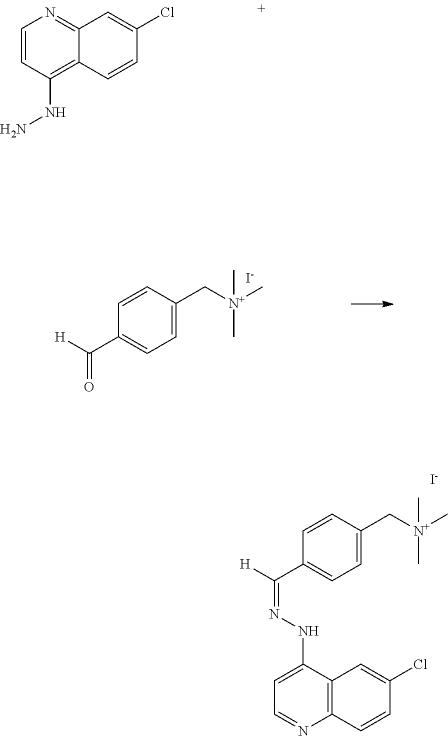 [SMILES: NNc1ccnc2cc(Cl)ccc12 ; C[N+](C)(C)Cc1ccc(C=O)cc1 ; [I-] ; [I-] ; C[N+](C)(C)Cc1ccc(/C=N\Nc2ccnc3ccc(Cl)cc23)cc1]

To a suspension of 7-chloro-4-hydrazinylquinoline (0.01 mol) in absolute ethanol (20 ml) and 1-(4-formylphenyl)—N,N,N-trimethylmethanaminium iodide (0.01 mol), NEt₃ (0.97 g, 0.01 mol) were added. The reaction mixture was refluxed for 8 h. The solution was evaporated under reduced pressure, and the crude mixture was purified by HPLC Method A. Yield: 36%.

¹H NMR (400 MHz, DMSO-d₆) δ 12.12 (s, NH), 8.73-7.16 (m, 10H), 4.60 (s, 2H), 3.07 (s, 6H).

LCMS Rₜ=0.85 min using Method 1, MS ES calcd. for C₂₀H₂₂ClN₄ [M+H]⁺ 354.9, found 353.2.

### Example 5: Preparation of (E)-4-(2-(4-(1H-imidazol-1-yl)benzylidene)hydrazinyl)-7-fluoroquinoline (5)

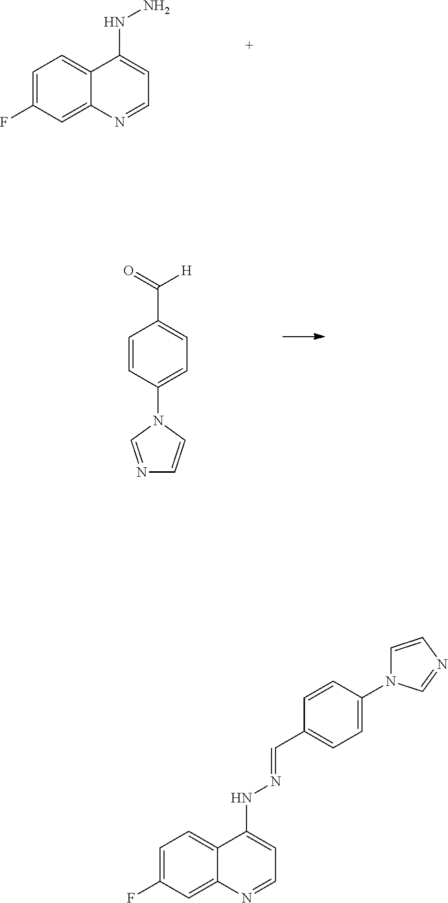 [SMILES: NNc1ccnc2cc(F)ccc12 ; O=Cc1ccc(-n2ccnc2)cc1 ; Fc1ccc2c(N/N=C/c3ccc(-n4ccnc4)cc3)ccnc2c1]

Same conditions as compound 10 using 7-fluoro-4-hydrazinylquinoline (1 mmol) and 4-(1H-imidazol-1-yl)benzaldehyde. Yield: 39%

¹H NMR (400 MHz, DMSO-d₆) δ 11.33 (s, 1H), 8.44 (s, 2H), 8.36 (s, 2H), 7.93 (d, J=8.3 Hz, 2H), 7.84 (s, 1H), 7.76 (d, J=8.1 Hz, 2H), 7.65-7.23 (m, 3H), 7.15 (s, 1H).

LCMS Rₜ=0.78 min using Method 1, MS ES-API calcd. for C₁₉H₁₄FN₅ [M+H]⁺ 332.4, found 331.1.

### Example 6: Preparation of (E)-4-(4-((2-(7-fluoroquinolin-4-yl)hydrazono)methyl)benzyl)morpholine (6)

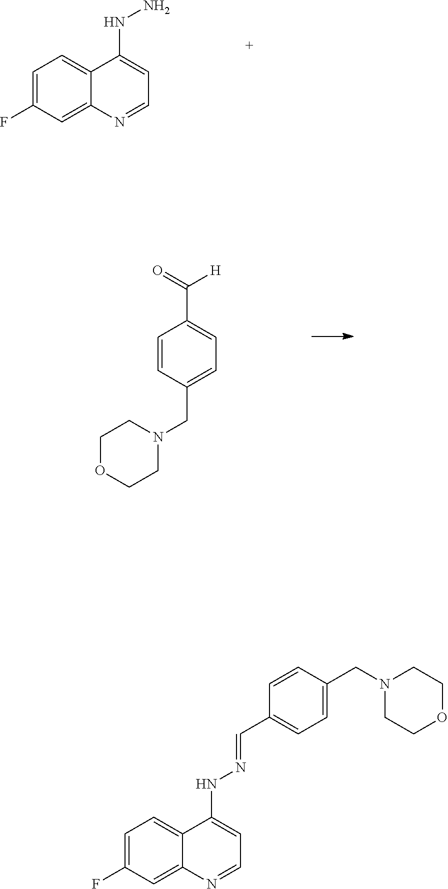 [SMILES: NNc1ccnc2cc(F)ccc12 ; O=Cc1ccc(CN2CCOCC2)cc1 ; Fc1ccc2c(N/N=C/c3ccc(CN4CCOCC4)cc3)ccnc2c1]

Same conditions as compound 10 using 7-fluoro-4-hydrazinylquinoline (1 mmol) and 4-(morpholinomethyl)benzaldehyde. Solid Yield: 49%

¹H NMR (400 MHz, DMSO-d₆) δ 12.96 (s, 1H), 8.86 (dd, J=9.4, 5.8 Hz, 1H), 8.78 (s, 1H), 8.58 (d, J=6.4 Hz, 1H), 7.84 (d, J=7.8 Hz, 2H), 7.73 (dd, J=9.9, 2.6 Hz, 1H), 7.59 (dd, J=19.8, 8.3 Hz, 3H), 7.50 (d, J=6.4 Hz, 1H), 3.86 (s, 2H), 3.70 (s, 4H), 2.69 (s, 4H).

LCMS Rₜ=0.76 min using Method 1, MS ES-API calcd. for C₂₁H₂₁FN₄O [M+H]⁺ 365.4, found 364.2.

### Example 7: Preparation of (E)-7-chloro-4-(2-(4-((4-methylpiperazin-1-yl)methyl)benzylidene)hydrazinyl)quinolone (7)

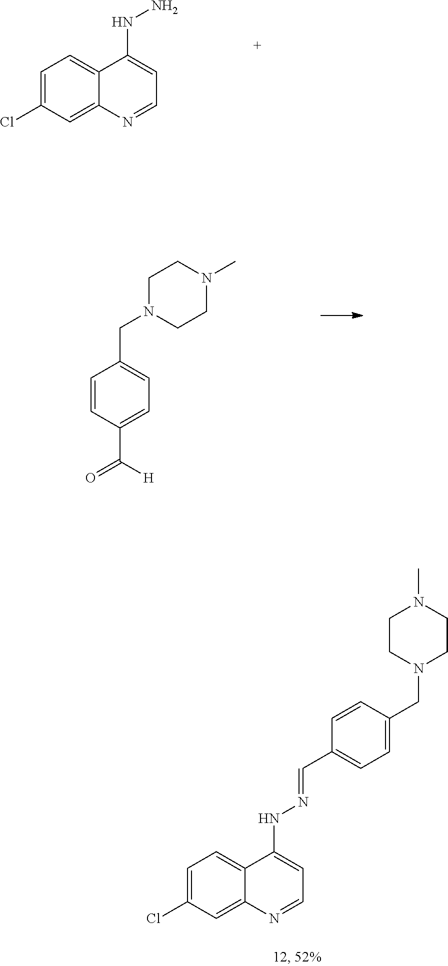 [SMILES: NNc1ccnc2cc(Cl)ccc12 ; CN1CCN(Cc2ccc(C=O)cc2)CC1 ; CN1CCN(Cc2ccc(/C=N/Nc3ccnc4cc(Cl)ccc34)cc2)CC1]

Same conditions as compound 10 using 7-chloro-4-hydrazinylquinoline (1 mmol) and 4-((4-methylpiperazin-1-yl)methyl)benzaldehyde to provide the product as a solid. Yield: 52%

¹H NMR (400 MHz, DMSO-d₆) δ 11.27 (s, 1H), 8.56 (s, 3H), 8.39 (d, J=11.2 Hz, 3H), 7.63 (ddd, J=145.2, 64.6, 11.9 Hz, 4H), 3.38 (s, 2H), 2.45 (s, 8H), 2.25 (s, 3H).

LCMS Rₜ=0.85 min using Method 1, MS ES-API calcd. for C₂₂H₂₄C₁N₅ [M+H]⁺ 394.9, found 393.2.

### Example 8: Preparation of (E)-1-(4-((2-(1,2-dihydroacenaphthylen-5-yl)hydrazineylidene)methyl)phenyl)-1H-imidazole (8)

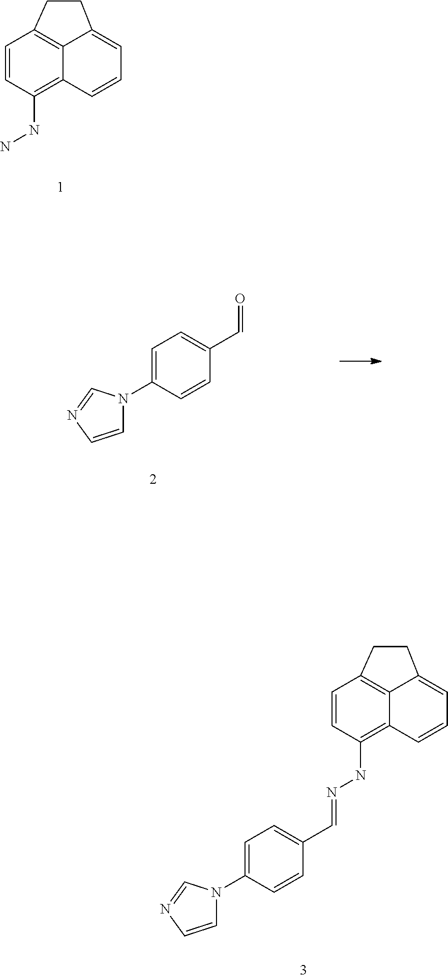 [SMILES: NNc1ccc2c3c(cccc13)CC2 ; O=Cc1ccc(-n2ccnc2)cc1 ; C(=N\Nc1ccc2c3c(cccc13)CC2)/c1ccc(-n2ccnc2)cc1]

(1,2-Dihydroacenaphthylen-5-yl)hydrazine (1 mmol) and 4-(1H-imidazol-1-yl)benzaldehyde (1 mmol) were dissolved in 0.5 ml DMSO, 10 mg CH₃COOH was added, heated at 100° C. for 30 min. Then mixture was cooled, 3 ml MeOH and 0.2 g of C-18 chromatographic phase were added, stirred for 2 hours, filtered and the solvent was evaporated. The residue was purified by HPLC Method A. Yield: 36%.

LCMS Rₜ=1.3 min using Method 1, MS ES-API calcd. for C₂₂H₁₈N₄ [M+H]⁺ 338.1, found 339.2.

### Example 9: Preparation of (Z)-1-methyl-4-((2-(quinolin-4-yl)hydrazono)methyl)pyridin-1-ium iodide (9)

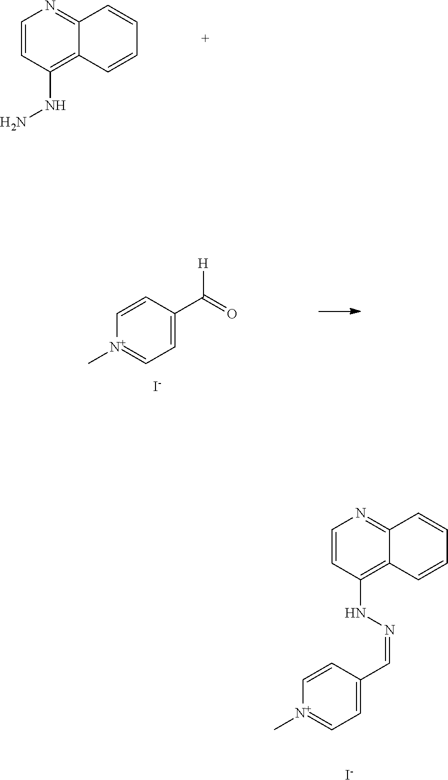

To a suspension of 4-hydrazinylquinoline (0.1 mmol) in absolute ethanol (20 mL) and 4-formyl-1-methylpyridin-1-ium iodide (0.1 mmol), NEt3 (0.97 g, 0.1 mmol) were added. The reaction mixture was refluxed for 8 h. The solution was evaporated under reduced pressure, and the crude mixture was purified by HPLC. Yield: 41%

¹H NMR (400 MHz, DMSO-d₆) δ 12.13 (s, 1H), 8.84 (d, J=6.4 Hz, 2H), 8.49 (s, 1H), 8.44 (d, J=8.4 Hz, 2H), 8.34 (d, J=6.4 Hz, 2H), 7.83-7.65 (m, 2H), 7.52 (d, J=10.2 Hz, 2H), 4.27 (s, 3H).

LCMS Rₜ=0.67 min using Method 1, MS ES-API calcd. for C₁₆H₁₅N₄ [M+H]⁺ 263.1, found 263.2.

### Example 10: Preparation of (E)-4-(4-((2-(7-fluoroquinolin-4-yl)hydrazono)methyl)benzyl)thiomorpholine 1,1-dioxide (10)

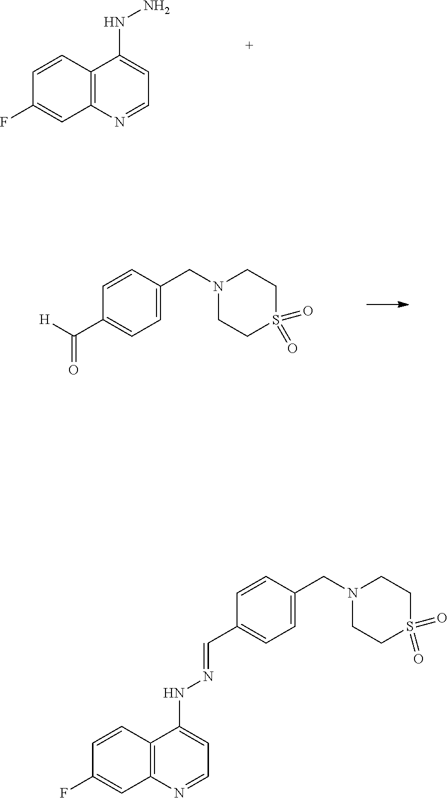 [SMILES: NNc1ccnc2cc(F)ccc12 ; O=Cc1ccc(CN2CCS(=O)(=O)CC2)cc1 ; O=S1(=O)CCN(Cc2ccc(/C=N/Nc3ccnc4cc(F)ccc34)cc2)CC1]

7-Fluoro-4-hydrazinylquinoline (1 mmol) and dioxidothiomorpholino)methyl)benzaldehyde (1 mmol) were dissolved in 0.5 ml of DMSO and then 10 mg of CH₃COOH was added, heated at 100° C. for 30 min. Then the reaction mixture was cooled, 3 mL of MeOH and 0.2 g of C-18 chromatographic phase were added and the mixture was stirred for 2 hours, filtered and the solvent evaporated. The residue was purified using HPLC Method A. Yield: 43%.

¹H NMR (400 MHz, DMSO-d₆) δ 8.75-7.33 (m, 6H), 3.72 (m, 2H), 3.11-2.99 (m, 8H).

LCMS Rₜ=0.99 min using Method 1, MS ES-API calcd. for C₂₁H₂₁FN₄O₂S [M+H]⁺ 413.5, found 412.2.

### Example 11: Preparation of 4-(2-(4-(1H-imidazol-1-yl)-3-methylbenzylidene)hydrazineyl)-6,7-dimethoxyquinoline hydrochloride (11)

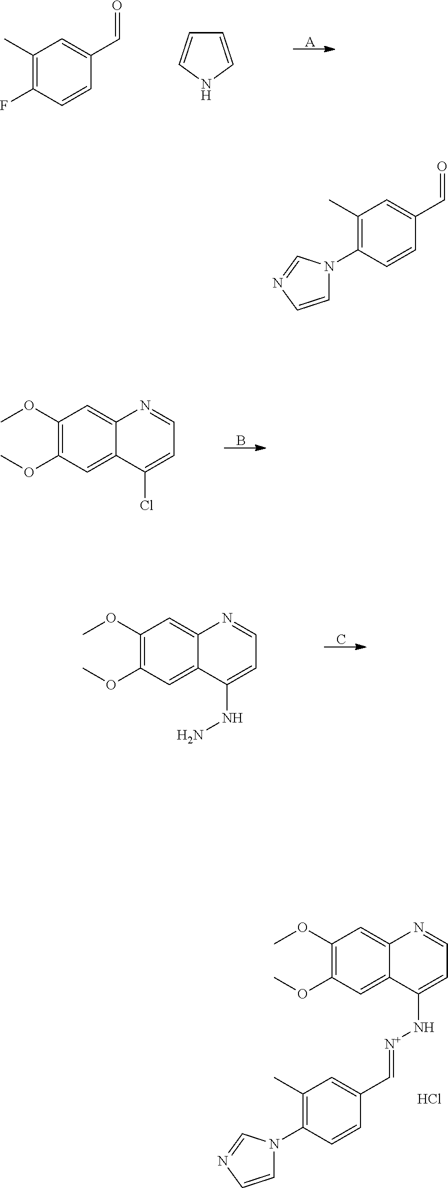

### Step A: Preparation of 4-(1H-imidazol-1-yl)-3-methylbenzaldehyde

4-fluoro-3-methylbenzaldehyde (1 mmol) and imidazole (1.3 mmol) were dissolved in 5 mL of dry DMSO, K₂CO₃ (3 mmol) was added, heated at 90° C. for 48 h. Then the reaction mixture was cooled, and 50 mL of water was added, stirred for 1 hour, filtered and washed with water (3×25 mL). The crude target compound was purified by HPLC. Yield: 36%.

### Step B: Preparation of 4-hydrazineyl-6,7-dimethoxyquinoline

To a suspension of 4-chloro-6,7-dimethoxyquinoline (1 mmol) in anhydrous dioxane (10 mL) hydrazine hydrate (2.2 mmol) was added. The reaction mixture was refluxed for 24 h. The solvent was evaporated, and the residue was treated with 20 mL of iPrOH to give a yellow solid. The crude target compound was purified by HPLC. Yellow solid. Yield: 44%.

### Step C: 4-(2-(4-(1H-imidazol-1-yl)-3-methylbenzylidene)hydrazineyl)-6,7-dimethoxyquinoline HCl salt

4-hydrazineyl-6,7-dimethoxyquinoline (1 mmol), 4-(1H-imidazol-1-yl)-3-methylbenzaldehyde (1 mmol) were dissolved in 1 mL of DMSO and then 10 mg of CH₃COOH was added, heated at 100° C. for 30 min. Then the reaction mixture was cooled and 3 mL of MeOH, 0.2 g of C-18 chromatographic phase and HCl conc. (3 mmol—solution in dioxane) were added, stirred for 2 hours, filtered and the solvent evaporated. The residue was purified using HPLC Method A. Yield: 82%.

¹H NMR (400 MHz, DMSO-d₆) δ 14.63 (s, 1H), 13.48 (s, 1H), 9.49 (s, 1H), 9.23 (s, 1H), 8.49 (d, J=6.6 Hz, 2H), 8.09 (s, 1H), 7.92 (d, J=12.3 Hz, 2H), 7.84 (d, J=7.6 Hz, 1H), 7.61 (dd, J=14.3, 7.5 Hz, 2H), 7.51 (s, 1H), 4.06 (s, 3H), 3.95 (s, 3H), 2.29 (s, 3H).

LCMS Rₜ=0.766 min using Method 1, MS ES-API calcd. for C₂₂H₂₁N₅O₂ [M+H]⁺ 388.2, found 387.19.

### Example 12: Preparation of 4-(2-(4-(1H-imidazol-1-yl)-3-methylbenzylidene)hydrazineyl)-7-methoxyquinoline (12)

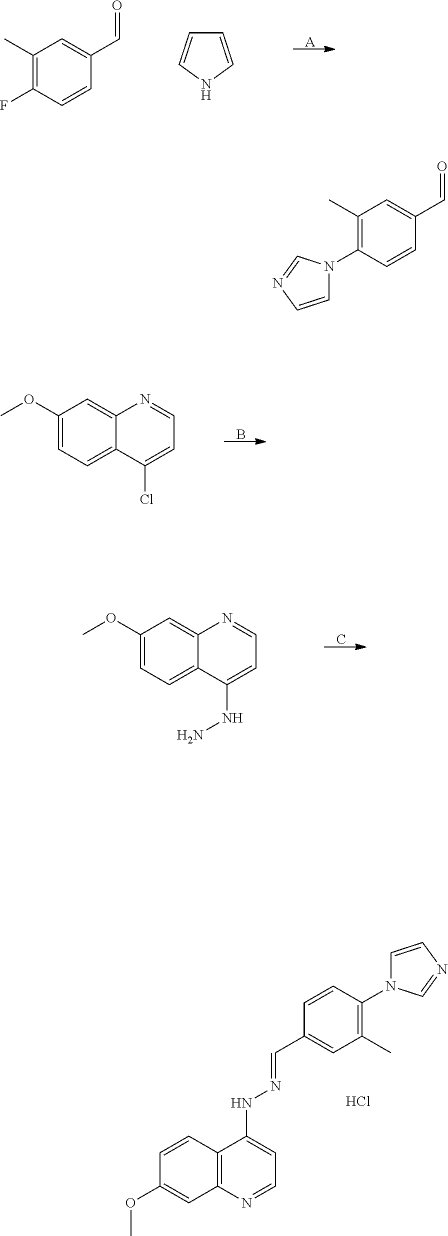

Step A: Preparation of 4-(1H-imidazol-1-yl)-3-methylbenzaldehyde: 4-fluoro-3-methylbenzaldehyde (1 mmol) and imidazole (1.3 mmol) were dissolved in 5 mL of dry DMSO, K₂CO₃ (3 mmol) was added, heated at 90° C. for 48 h. Then the reaction mixture was cooled, and 50 mL of water was added, stirred for 1 hour, filtered and washed with water (3×25 mL). The crude target compound was purified by LC. Yellow solid. Yield: 18%.

Step B: Preparation of 4-hydrazineyl-7-methoxyquinoline: To a suspension of 4-chloro-7-methoxyquinoline (1 mmol) in anhydrous dioxane (10 mL) hydrazine hydrate (2.2 mmol) was added. The reaction mixture was refluxed for 24 h. The solvent was evaporated, and the residue was treated with 20 mL of iPrOH to give a yellow solid. The crude target compound was purified by HPLC. Yield: 54%.

Step C: Preparation of 4-(2-(4-(1H-imidazol-1-yl)-3-methylbenzylidene)hydrazineyl)-7-methoxyquinoline: 4-Hydrazineyl-7-methoxyquinoline (1 mmol) and 4-(1H-imidazol-1-yl)-3-methylbenzaldehyde (1 mmol) were dissolved in 1 mL of DMSO and then 10 mg of CH₃COOH was added, heated at 100° C. for 30 min. Then the reaction mixture was cooled and 3 mL of MeOH, 0.2 g of C-18 chromatographic phase and HCl conc. (3 mmol—solution in dioxane) were added, stirred for 2 hours, filtered, evaporated. The residue was purified using HPLC Method A. Yield: 87%.

¹H NMR (400 MHz, DMSO-d₆) δ 14.62 (s, 1H), 13.28 (s, 1H), 9.47 (s, 1H), 9.05 (s, 1H), 8.97 (d, J=9.4 Hz, 1H), 8.60 (d, J=7.0 Hz, 1H), 8.08 (s, 1H), 7.98 (s, 1H), 7.93 (d, J=1.9 Hz, 1H), 7.91 (s, 1H), 7.66 (d, J=8.2 Hz, 1H), 7.63 (d, J=6.9 Hz, 1H), 7.50 (d, J=2.6 Hz, 1H), 7.43 (dd, J=9.4, 2.5 Hz, 1H), 3.97 (s, 3H), 2.30 (s, 3H).

LCMS Rₜ=0.75 min using Method 1, MS ES-API calcd. for C₂₁H₁₉N₅O [M+H]⁺ 358.4, found 357.2.

### Example 13: Preparation of (E)-4-(2-(4-(1H-imidazol-1-yl)-3-methoxybenzylidene)hydrazineyl)-6,7-dimethoxyquinoline Phosphoric Acid Salt (13)

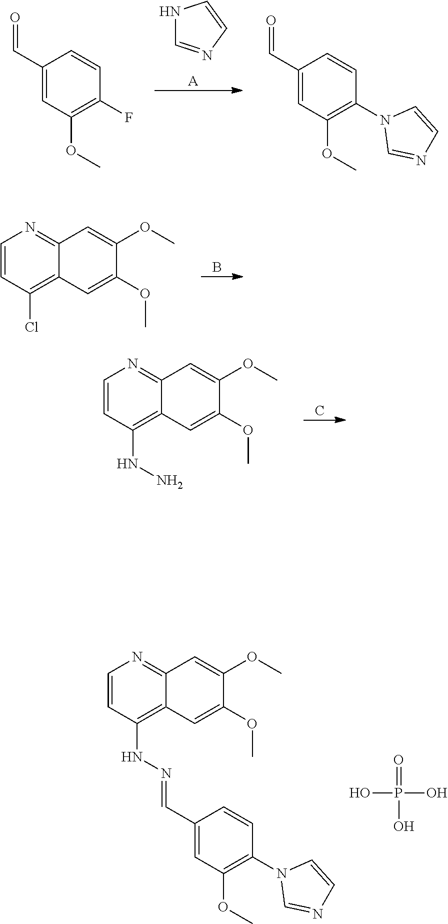

### Step A: Preparation of 4-(1H-imidazol-1-yl)-3-methoxybenzaldehyde

4-Fluoro-3-methoxybenzaldehyde (1 mmol) and imidazole (1.3 mmol) were dissolved in 5 ml of dry DMSO, K₂CO₃ (3 mmol) was added, heated at 90° C. for 48 h. Then the mixture was cooled, and 50 ml of water was added, the mixture was stirred for 1 hour, the precipitate was filtered and washed with water (3×25 ml). The crude target, 4-(1H-imidazol-1-yl)-3-methoxybenzaldehyde was purified by HPLC. Yield: 61%.

### Step B. Preparation of 4-hydrazineyl-6,7-dimethoxyquinoline

To a suspension of 4-chloro-6,7-dimethoxyquinoline (1 mmol) in anhydrous dioxane (10 ml) hydrazine hydrate (2.2 mmol) was added. The reaction mixture was refluxed for 24 h. The solvent was evaporated, and the residue was treatment with 20 ml of iPrOH to give a yellow solid. The crude compound was purified by LC. Yield: 59%.

### Step C: Preparation of (E)-4-(2-(4-(1H-imidazol-1-yl)-3-methoxybenzylidene)hydrazineyl)-6,7-dimethoxyquinoline phosphoric acid salt

4-Hydrazineyl-6,7-dimethoxyquinoline (1 mmol) and 4-(1H-imidazol-1-yl)-3-methoxybenzaldehyde (1 mmol) were dissolved in 1 ml DMSO, 10 mg CH₃COOH was added, heated at 100° C. for 30 min. Then mixture was cooled and 3 ml MeOH, 0.2 g of C-18 chromatographic phase and H₃PO₄ (3 mmol) were added, stirred for 2 hours, filtered, evaporated. The residue was purified using HPLC Method A. Yield: 72%.

¹H NMR (400 MHz, DMSO-d₆) δ 11.11 (s, 1H), 8.41 (s, 1H), 8.41-7.07 (m, 10H), 3.94 (s, 6H), 3.9 (s, 3H).

LCMS Rₜ=0.89 min using Method 1, MS ES-API calcd. for C₂₂H₂₁N₅O₃ [M+H]⁺ 404.4, found 403.2.

### Example 14: Preparation of (E)-4-(2-((6-(1H-imidazol-1-yl)pyridin-3-yl)methylene)hydrazineyl)-7-chloroquinoline HCl salt (14)

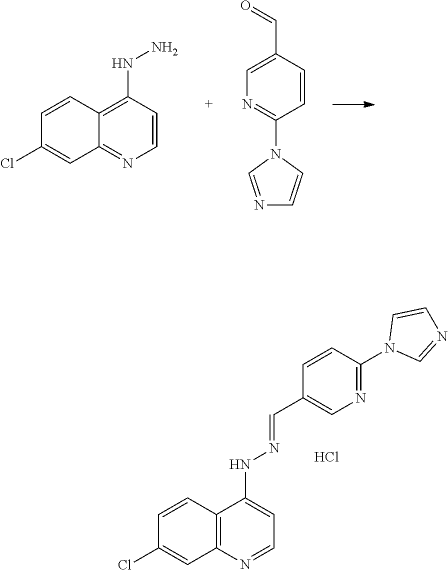

Step A: 7-chloro-4-hydrazineylquinoline (1 mmol), 6-(1H-imidazol-1-yl)nicotinaldehyde (1 mmol) were dissolved in 1 mL of DMSO, 10 mg CH₃COOH was added, heated at 100° C. for 30 min. Then the reaction mixture was cooled and 3 mL of MeOH, 0.2 g of C-18 chromatographic phase and HCl (3 mmol—solution in dioxane) were added, stirred for 2 hours, filtered, evaporated. The residue was purified using HPLC Method A to provide the product as a solid Yield: 72%.

¹H NMR (400 MHz, DMSO-d₆) δ 13.69 (s, 1H), 9.95 (s, 1H), 9.11 (d, J=10.5 Hz, 2H), 8.98 (s, 1H), 8.68 (dd, J=29.1, 7.8 Hz, 2H), 8.49 (s, 1H), 8.18 (d, J=10.5 Hz, 2H), 7.88 (s, 1H), 7.80 (dd, J=25.1, 8.1 Hz, 2H), 3.56 (s, 1H).

LCMS Rₜ=0.80 min using Method 1, MS ES-API calcd. for CisH13ClN6 [M+H]⁺ 349.8, found 348.1.

### Example 15: Preparation of 7-chloro-4-(2-((6-(4,5-dimethyl-1H-imidazol-1-yl)pyridin-3-yl)methylene)hydrazineyl)quinazoline hydrochloride (15)

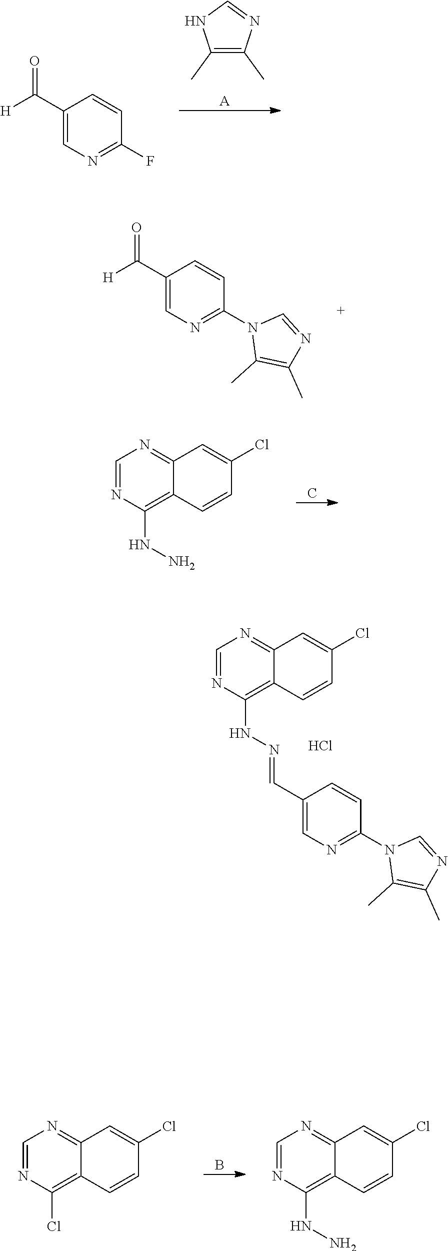

### Step A: Preparation of 6-(4,5-dimethyl-1H-imidazol-1-yl)nicotinaldehyde

6-Fluoronicotinaldehyde (1 mmol) and 4,5-dimethyl-1H-imidazole (1.3 mmol) were dissolved in 5 mL of dry DMSO then K₂CO₃ (3 mmol) was added and the mixture was heated at 90° C. for 48 h. Then the reaction mixture was cooled, and 50 mL of water was added, and stirred for 1 hour, filtered and washed with water (3×25 mL). The crude target compound was purified by recrystallization from a mixture of DMF/iPrOH (1/5). Yellow solid. Yield: 41%.

### Step B: Preparation of 7-chloro-4-hydrazineylquinazoline

To a suspension of 4,7-dichloroquinazoline (1 mmol) in anhydrous THE (10 mL) hydrazine hydrate (1.8 mmol) was added. The reaction mixture was stirred at room temperature for 18 h. The solvent was partially evaporated at room temperature, and the precipitate that formed was filtered and washed with cold THE to give a yellow solid. The crude target compound was purified by LC. Yellow solid. Yield: 49%.

### Step C: Preparation of (E)-7-chloro-4-(2-((6-(4,5-dimethyl-1H-imidazol-1-yl)pyridin-3-yl)methylene)hydrazineyl)quinazoline

6-(4,5-Dimethyl-1H-imidazol-1-yl)nicotinaldehyde (1 mmol), 7-chloro-4-hydrazineylquinazoline (1 mmol) were dissolved in 1 mL DMSO, 10 mg CH₃COOH was added and the mixture was heated at 100° C. for 30 min. Then mixture was cooled and 3 mL of MeOH, 0.2 g of C-18 chromatographic phase and HCl conc. (3 mmol—solution in dioxane) were added, stirred for 2 hours, filtered, evaporated. The residue was purified using HPLC Method A to provide the product as a solid. Yield: 40%.

¹H NMR (400 MHz, DMSO-d₆) δ 11.95 (s, 1H), 9.10-7.23 (m, 9H), 2.33 (s, 3H), 2.12 (s, 3H).

LCMS Rₜ=1.05 min using Method 1, MS ES-API calcd. for C₁₉H₁₆ClN₇ [M+H]⁺ 378.8, found 377.1.

### Example 16: Preparation of 7-chloro-4-(2-(1-(6-(4,5-dimethyl-1H-imidazol-1-yl)pyridin-3-yl)ethylidene)hydrazineyl)quinazoline phosphoric acid salt (16)

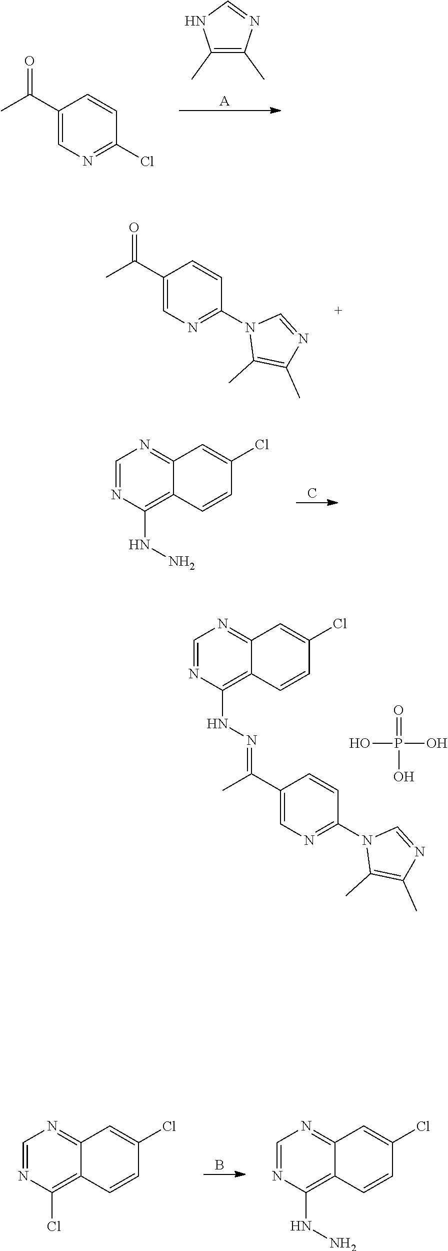

### Step A: Preparation of 1-(6-(4,5-dimethyl-1H-imidazol-1-yl)pyridin-3-yl)ethan-1-one

To a solution of 1-(6-chloropyridin-3-yl)ethan-1-one (1 mmol) and 4,5-dimethyl-1H-imidazole (1.1 mmol) in dry pyridine (10 mL) was heated at 90° C. for 12-15 h. After the completion, the reaction mixture was cooled, 50 mL of water was added, and the mixture was stirred for 1 h at room temperature. The precipitate was filtered, washed with water (3×25 mL) and dried. The crude target compound was purified by recrystallization from mixture of DMF/iPrOH (1/1). Yield: 54%.

### Step B: Preparation of 7-chloro-4-hydrazineylquinazoline

To a suspension of 4,7-dichloroquinazoline (1 mmol) in anhydrous THE (10 mL) was added hydrazine hydrate (1.8 mmol). The reaction mixture was stirred at room temperature for 18 h. The solvent was partially evaporated at room temperature and the precipitate that was formed, was filtered and washed with cold THE to give a yellow solid. The crude target compound was purified by HPLC. Yield: 49%.

### Step C: Preparation of (E)-7-chloro-4-(2-(1-(6-(4,5-dimethyl-1H-imidazol-1-yl)pyridin-3-yl)ethylidene)hydrazineyl)quinazoline phosphoric acid salt

1-(6-(4,5-Dimethyl-1H-imidazol-1-yl)pyridin-3-yl)ethan-1-one (1 mmol), 7-chloro-4-hydrazineylquinazoline (1 mmol) were dissolved in 1 mL DMSO, 10 mg CH₃COOH was added and the mixture was heated at 100° C. for 30 min. Then mixture was cooled and 3 mL of methanol, 0.2 g of C-18 chromatographic phase and H₃PO₄ (2 mmol) were added, stirred for 2 hours, filtered and evaporated. The residue was purified using HPLC Method A to provide a solid. Yield: 40%.

¹H NMR (400 MHz, DMSO-d₆) δ 11.73 (s, 1H), 9.28-7.38 (m, 8H), 2.55 (s, 3H), 2.33 (s, 3H), 2.13 (s, 3H).

LCMS Rₜ=1.2 min using Method 1, MS ES-API calcd. for C₂₀H₁₈ClN₇ M+H]⁺392.9, found 391.2.

### Example 17: Preparation of (E)-1-(5-(1-(2-(7-chloroquinazolin-4-yl)hydrazineylidene)ethyl)pyridin-2-yl)-3-methyl-1H-imidazol-3-ium hydrogen iodide phosphoric acid salt (17)

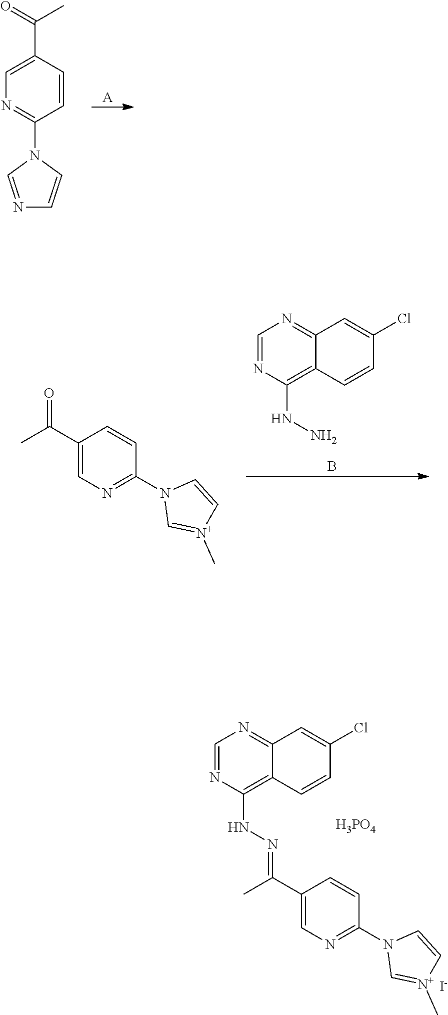

### Step A: Preparation of 1-(5-acetylpyridin-2-yl)-3-methyl-1H-imidazol-3-ium iodide

1-(6-(1H-Imidazol-1-yl)pyridin-3-yl)ethan-1-one (1 mmol) and Mel (20 mmol) were dissolved in 10 ml of dry toluene and the mixture was heated with reflux condenser for 64 h with stirring. Then the reaction mixture was cooled, filtered and precipitate was treatment with toluene (20 ml) and stirred for 2 h at room temperature. The precipitate was filtered, washed with toluene (3×20 ml) and dried. Yield: 58%.

### Step B: Preparation of (E)-1-(5-(1-(2-(7-chloroquinazolin-4-yl)hydrazineylidene)ethyl)pyridin-2-yl)-3-methyl-1H-imidazol-3-ium iodide phosphoric acid salt

1-(5-acetylpyridin-2-yl)-3-methyl-1H-imidazol-3-ium iodide 1 mmol) and 7-chloro-4-hydrazineylquinazoline (1 mmol) were dissolved in 1 ml DMSO, 10 mg CH₃COOH was added, heated at 100° C. for 30 min. Then mixture was cooled and 3 ml MeOH was added and then the mixture was stirred for 2 hours, filtered, evaporated. The residue was dissolved in the mixture of DMF/iPrOH (1/1), H₃PO₄ (2 mmol) was added, stirred for 2 h at 50° C., cooled, filtered, washed with iPrOH and dried. Yield: 38%.

¹H NMR (400 MHz, DMSO-d₆) δ 11.80 (s, 1H), 10.09 (s, 1H), 9.30 (s, 1H), 8.83 (d, J=8.7 Hz, 1H), 8.56 (s, 1H), 8.27 (d, J=8.6 Hz, 1H), 8.15-7.85 (m, 3H), 7.61-7.35 (m, 2H), 4.01 (s, 3H), 2.69-2.52 (m, 4H).

LCMS Rₜ=1.0 min using Method 1, MS ES-API calcd. for C₁₉H₁₈ClN₇ [M+H]⁺ 379, found 379.

### Example 18: Preparation of (E)-7-chloro-4-(2-(1-(6-(2,4-dimethyl-1H-imidazol-1-yl)pyridin-3-yl)ethylidene)hydrazineyl)quinazoline phosphoric acid salt (18)

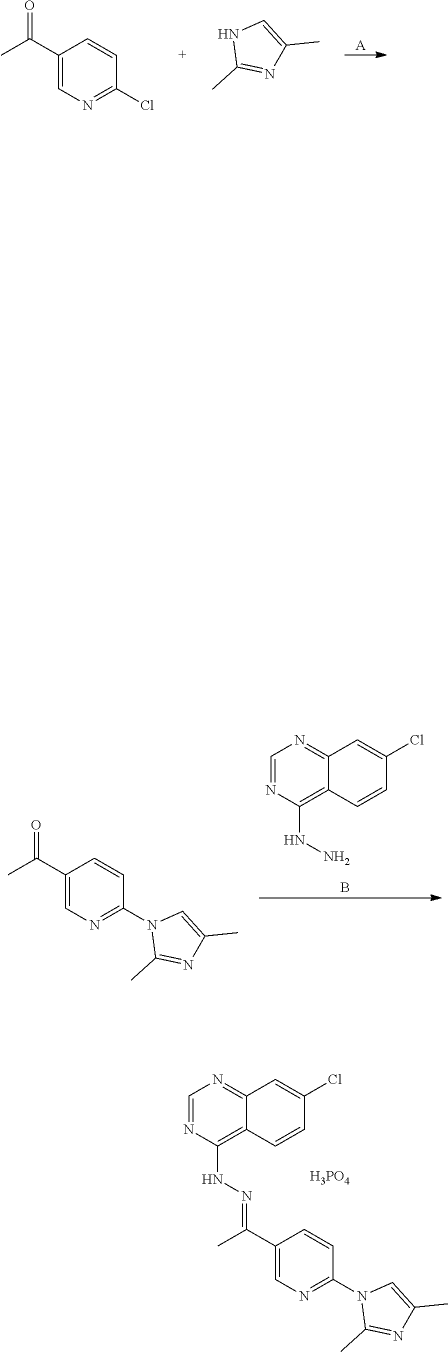

### Step A: Preparation of 1-(6-(2,4-dimethyl-1H-imidazol-1-yl)pyridin-3-yl)ethan-1-one

1-(6-Chloropyridin-3-yl)ethan-1-one (1 mmol) and 2,4-dimethyl-1H-imidazole (1.1 mmol) were dissolved in 5 ml of dry DMSO and K₂CO₃ (3 mmol) was added. The mixture was heated at 90° C. for 7 days. Then the mixture was cooled, and 50 ml of water was added, and the mixture was stirred for 2 h at r.t. The precipitate that formed was filtered, washed with water (3×25 ml) and dried. The crude target compound was purified by recrystallization from a mixture of DMF/iPrOH (1/5). Yellow solid. Yield: 42%.

### Step B: Preparation of (E)-7-chloro-4-(2-(1-(6-(2,4-dimethyl-1H-imidazol-1-yl)pyridin-3-yl)ethylidene)hydrazineyl)quinazoline phosphoric acid salt

1-(6-(2,4-Dimethyl-1H-imidazol-1-yl)pyridin-3-yl)ethan-1-one (1 mmol) and 7-chloro-4-hydrazineylquinazoline (1 mmol) were dissolved in 1 ml DMSO. Into this mixture 10 mg CH₃COOH was added and then the mixture was heated at 100° C. for 1 h. Then the mixture was cooled, and 3 ml MeOH, 0.2 g of C-18 chromatographic phase and H₃PO₄ (2 mmol) was added there and was stirred for 2 h at r.t. The precipitate was filtered, and the remaining solution was evaporated. The residue was purified using HPLC Method A. Yield: 39%.

1H NMR (400 MHz, DMSO-d₆) δ 2.11 (3H, s, CH₃), 2.59 (6H, s, 2CH₃), (9.25-7.4, m, Ar), 11.75 (NH, s).

LCMS Rt=1.07 min using Method 1, MS ES-API calcd. for C₂₀H₂₁ClN₇O₄P [M+H]⁺ 489.9, found 391.2.

### Example 19: Preparation of (E)-4-(2-(1-(6-(2,4-dimethyl-1H-imidazol-1-yl)pyridin-3-yl)ethylidene)hydrazineyl)quinazoline (19)

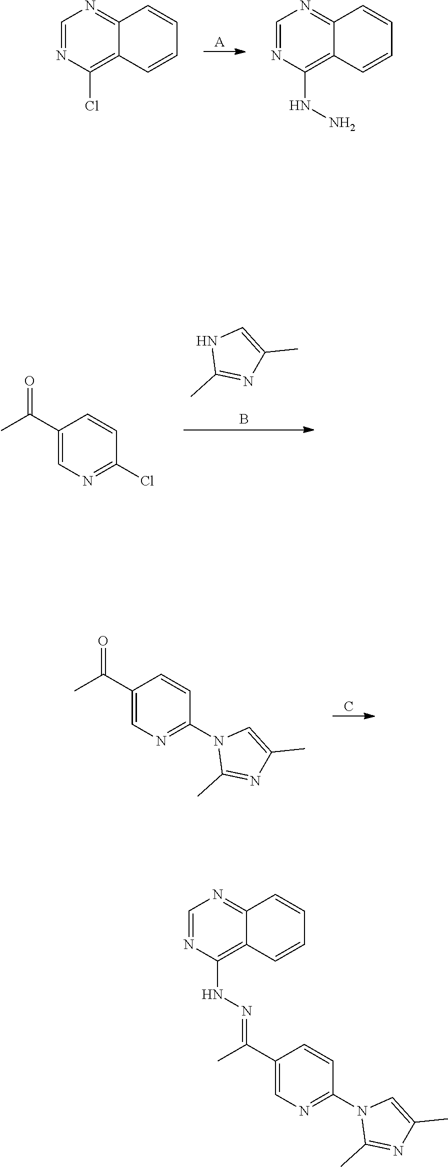

### Step A: Preparation of 4-hydrazineylquinazoline

To a suspension of 4-chloroquinazoline (2 mmol) in anhydrous THE (10 ml) hydrazine hydrate (2.2 mmol) was added. The reaction mixture was stirred at room temperature for 18 h. The solvent volume was reduced by half with evaporation at r.t., The precipitate that was formed, filtered and washed with cold THE to give a yellow solid. The crude target compound was purified by HPLC. Yield: 56%.

### Step B: Preparation of 1-(6-(2,4-dimethyl-1H-imidazol-1-yl)pyridin-3-yl)ethan-1-one

To a solution of 1-(6-chloropyridin-3-yl)ethan-1-one (1 mmol) and 2,4-dimethyl-1H-imidazole (1.1 mmol) in dry pyridine (10 mL) was heated at 90° C. for 12-15 h with stirring. The reaction mixture was cooled and then 50 ml of water was added, and the mixture was stirred for 1 h at r.t.

The precipitate was filtered, washed with water (3×25 ml) and dried. The crude target compound was purified by recrystallized from mixture of DMF/iPrOH (1/1). Yield: 72%.

### Step C: Preparation of (E)-4-(2-(1-(6-(2,4-dimethyl-1H-imidazol-1-yl)pyridin-3-yl)ethylidene)hydrazineyl)quinazoline

1-(6-(2,4-dimethyl-1H-imidazol-1-yl)pyridin-3-yl)ethan-1-one (1 mmol) and 4-hydrazineylquinazoline (1 mmol) were dissolved in 1 ml DMSO, 10 mg CH₃COOH was added and the mixture was heated at 100° C. for 30 min. Then mixture was cooled and 3 ml MeOH, stirred for 2 hours, filtered, evaporated. The residue was purified using HPLC Method A to provide the product as a solid. Yield: 46%.

¹H NMR (400 MHz, CDCl₃) δ 10.40-10.19 (m, 3H), 8.93 (s, 4H), 8.36 (d, J=7.6 Hz, 4H), 8.22 (s, 3H), 7.85 (s, 4H), 7.70-7.50 (m, 7H), 7.42 (s, 5H), 7.26 (d, J=13.6 Hz, 14H), 7.03 (s, 3H), 2.59 (d, J=25.1 Hz, 19H), 2.27 (d, J=24.0 Hz, 10H), 1.58 (s, 16H), 1.23 (s, 8H).

LCMS Rₜ=0.97 min using Method 1, MS ES-API calcd. for C₂₀H₁₉N₇ [M+H]⁺ 358.2, found 358.2.

### Example 20: Preparation of 7-chloro-N-(2-(4-methylpiperazin-1-yl)pyridin-4-yl)quinolin-4-amine (20)

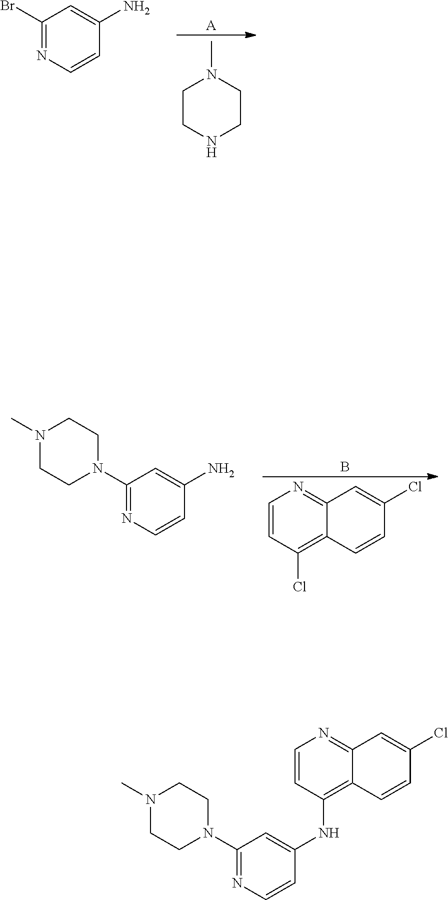

### Step A: Preparation of 2-(4-methylpiperazin-1-yl)pyridin-4-amine

2-bromopyridin-4-amine (1 mmol) and N-methyl piperazine (10 mmol) were heated at 140° C. for 24 h. Then the reaction mixture was evaporated and treated with iPrOH (20 mL) and stirred for 30 min at room temperature. The precipitate that formed was filtered, washed with cold iPrOH (3×20 mL) and dried. The crude target compound was purified by HPLC. Yellow solid. Yield: 22%.

### Step B: 7-chloro-N-(2-(4-methylpiperazin-1-yl)pyridin-4-yl)quinolin-4-amine

2-(4-methylpiperazin-1-yl)pyridin-4-amine (1 mmol) and 4,7-dichloroquinoline (1 mmol) were dissolved in 10 mL of dry acetic acid and the mixture was heated at 90° C. for 48 h. Then the reaction mixture was cooled, filtered and solvent was evaporated. The residue was treated with iPrOH (20 mL) and stirred for 30 min at room temperature. The precipitate was filtered, washed with cold iPrOH (3×20 mL) and dried. The crude target compound was purified by HPLC, Method A to provide a solid. Yield: 38%.

¹H NMR (400 MHz, DMSO-d₆) δ 9.23 (s, 1H), 8.62 (d, J=5.2 Hz, 1H), 8.35 (d, J=9.0 Hz, 1H), 8.01 (d, J=6.0 Hz, 1H), 7.96 (d, J=2.3 Hz, 1H), 7.62 (dd, J=9.1, 2.3 Hz, 1H), 7.31 (d, J=5.3 Hz, 1H), 6.65 (s, 1H), 3.44 (t, J=5.1 Hz, 4H), 2.54 (s, 1H), 2.39 (t, J=5.0 Hz, 4H), 2.21 (s, 3H).

LCMS Rₜ=0.56 min using Method 1, MS ES-API calcd. for C₁₉H₂₀N₅Cl [M+H]⁺ 354.2, found 354.2.

### Example 21: Preparation of (E)-4-(2-(1-(6-(4,5-dimethyl-1H-imidazol-1-yl)pyridin-3-yl)ethylidene)hydrazineyl)-7-fluoroquinazoline (21)

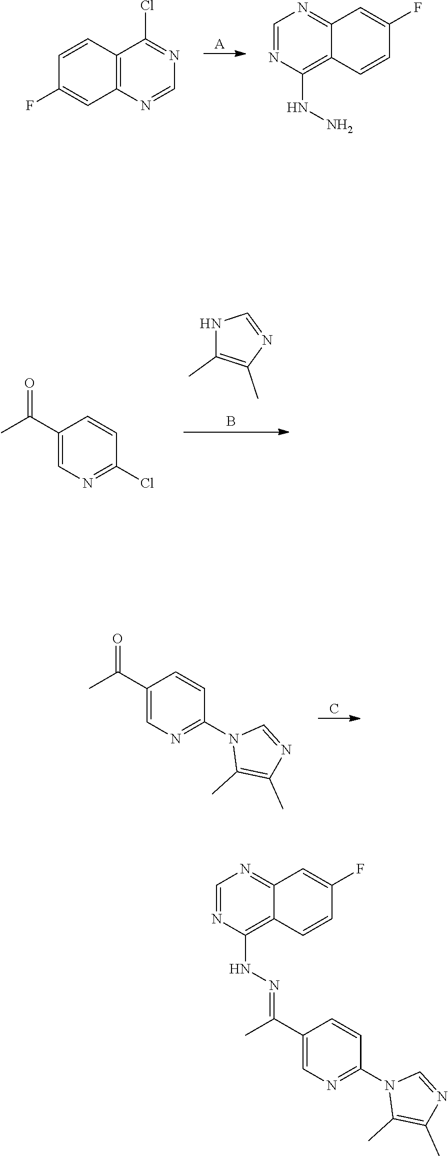

### Step A: Preparation of 7-fluoro-4-hydrazineylquinazoline

To a suspension of compound 4-chloro-7-fluoroquinazoline (2 mmol) in anhydrous THE (10 ml) was added hydrazine hydrate (2.2 mmol) and the reaction mixture was stirred at room temperature for 18 h. The solvent was partially evaporated at r.t., the precipitate that was formed was filtered and washed with cold THE to give a yellow solid. The crude target compound was purified by HPLC. Yield: 65%.

### Step B: Preparation of 1-(6-(4,5-dimethyl-1H-imidazol-1-yl)pyridin-3-yl)ethan-1-one

To a solution of compound 1 (2 mmol) and 2 (2.2 mmol) in dry pyridine (20 mL) was heated at 90° C. for 12-15 h with stirring. The reaction mixture was cooled, 200 ml of water was added, and the mixture was stirred for 1 h at r.t. The precipitate was filtered, washed with water (3×75 ml) and dried. The crude target compound was purified by recrystallization from mixture of DMF/iPrOH (1/1). Yield: 47%.

### Step C: Preparation of (E)-4-(2-(1-(6-(4,5-dimethyl-1H-imidazol-1-yl)pyridin-3-yl)ethylidene)hydrazineyl)-7-fluoroquinazoline

7-Fluoro-4-hydrazineylquinazoline (1 mmol), 1-(6-(4,5-dimethyl-1H-imidazol-1-yl)pyridin-3-yl)ethan-1-one (1 mmol) were dissolved in 1 ml DMSO, 10 mg CH₃COOH was added, heated at 100° C. for 30 min. Then mixture was cooled and 3 ml MeOH was added, stirred for 2 hours, filtered the precipitate, evaporated solvent. The residue was purified using HPLC Method A. Yield: 41%.

¹H NMR (400 MHz, DMSO-d₆) δ 11.78 (s, 1H), 9.35 (s, 1H), 8.75 (d, J=39.2 Hz, 2H), 8.35 (s, 1H), 7.86 (d, J=54.1 Hz, 2H), 7.31 (s, 2H), 2.57 (d, J=18.6 Hz, 3H), 2.29 (d, J=44.0 Hz, 7H).

LCMS Rₜ=1.0 min using Method 1, MS ES-API calcd. for C₂₀H₁₈FN₇ [M+H]⁺ 376.2, found 376.2.

### Example 22: Preparation of 7-chloro-4-(2-(1-(6-(4,5-dimethyl-1H-imidazol-1-yl)pyridin-3-yl)ethylidene)hydrazineyl)quinoline (22)

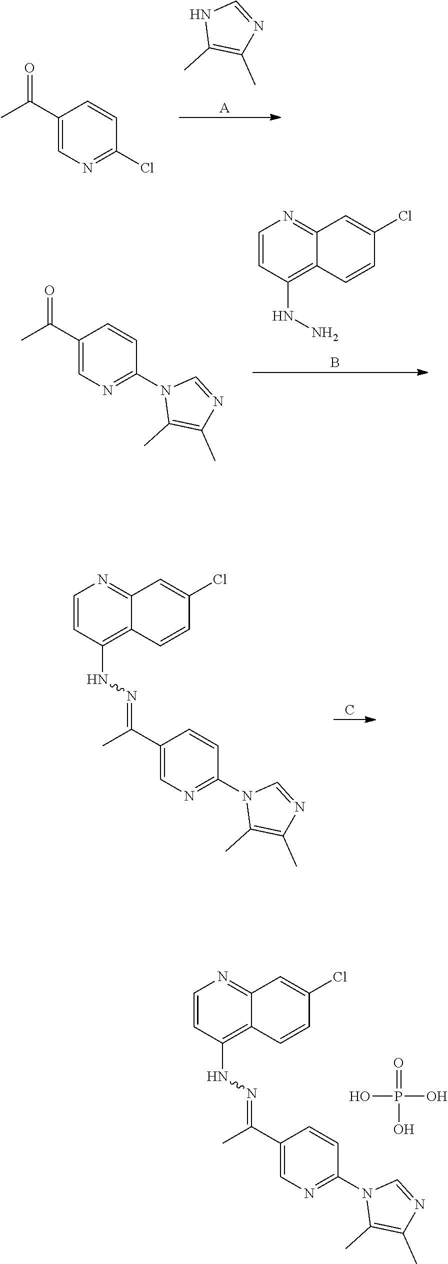

### Step A: Preparation of 1-(6-(4,5-dimethyl-1H-imidazol-1-yl)pyridin-3-yl)ethan-1-one

To a solution of 1-(6-chloropyridin-3-yl)ethan-1-one (4 mmol) and 4,5-dimethyl-1H-imidazole (4.4 mmol) in dry pyridine (40 mL) was heated at 90° C. for 12-15 h with stirring. The reaction mixture was cooled, 200 ml of water was added and the mixture was stirred for 1 h at r.t. The precipitate was filtered, washed with water (3×75 ml) and dried. The crude target compound was purified by recrystallization from a mixture of DMF/iPrOH (1/1). Yield: 47%.

### Step B: Preparation of 7-chloro-4-(2-(1-(6-(4,5-dimethyl-1H-imidazol-1-yl)pyridin-3-yl)ethylidene)hydrazineyl)quinoline

1-(6-(4,5-Dimethyl-1H-imidazol-1-yl)pyridin-3-yl)ethan-1-one (2 mmol) and 7-chloro-4-hydrazineylquinoline (2 mmol) were dissolved in 1 ml DMSO, 20 mg CH₃COOH was added, heated at 100° C. for 30 min. Then mixture was cooled and 7 ml MeOH was added and the mixture was, stirred for 2 hours, filtered, and the solvent was evaporated. The residue was purified using HPLC. Yield: 39%

### Step C: Preparation of 7-chloro-4-(2-(1-(6-(4,5-dimethyl-1H-imidazol-1-yl)pyridin-3-yl)ethylidene)hydrazineyl)quinoline Phosphoric acid salt

7-chloro-4-(2-(1-(6-(4,5-dimethyl-1H-imidazol-1-yl)pyridin-3-yl)ethylidene)hydrazineyl)quinoline (1 mmol) was dissolved in 3 ml/1 ml MeOH/H₃PO₄. The mixture was stirred for 2 hours, evaporated and the residue was dissolved in the mixture of DMF/iPrOH (1/1), stirred for 2 h at 50° C., cooled, filtered, washed with iPrOH and dried. Yield: 86%.

¹H NMR (400 MHz, DMSO-d₆) δ 9.03 (s, 1H), 8.44 (t, J=9.1 Hz, 2H), 8.03 (s, 1H), 7.64 (d, J=8.5 Hz, 2H), 7.40 (s, 1H), 7.07 (s, 1H), 2.54 (s, 3H), 2.31 (s, 3H), 2.13 (s, 3H).

LCMS Rₜ=0.80 min using Method 1, MS ES-API calcd. for C₂₁H₁₉ClN₆ [M+H]⁺ 391.1, found 391.0.

### Example 23: Preparation of 1-(5-(1-(2-(7-chloroquinolin-4-yl)hydrazineylidene)ethyl)pyridin-2-yl)-3-methyl-1H-imidazol-3-ium iodide (23)

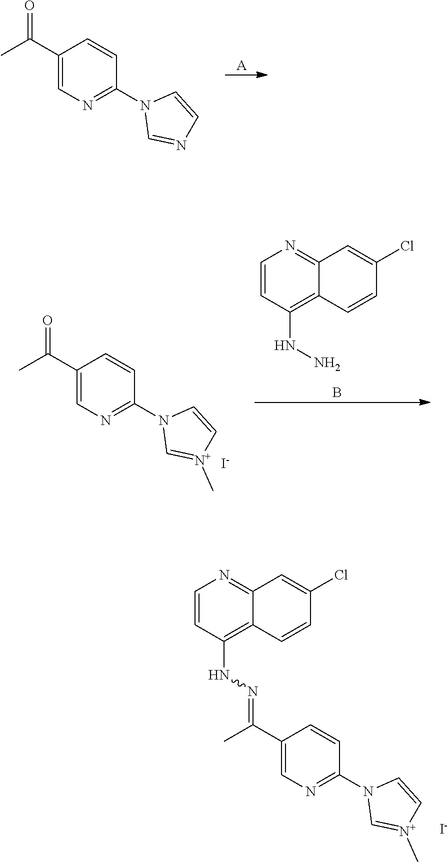

### Step A: Preparation of 1-(5-acetylpyridin-2-yl)-3-methyl-1H-imidazol-3-ium iodide

1-(6-(1H-Imidazol-1-yl)pyridin-3-yl)ethan-1-one (2 mmol) and Mel (40 mmol) were dissolved in 10 ml of dry toluene and the mixture was heated with reflux condenser for 64 h with stirring. Then the reaction mixture was cooled, filtered and precipitate was treatment with toluene (20 ml) and stirred for 2 h at room temperature. The precipitate that was formed was filtered, washed with toluene (3×20 ml) and dried. Yield: 41%.

### Step B: Preparation of 1-(5-(1-(2-(7-chloroquinolin-4-yl)hydrazineylidene)ethyl)pyridin-2-yl)-3-methyl-1H-imidazol-3-ium iodide

1-(5-Acetylpyridin-2-yl)-3-methyl-1H-imidazol-3-ium iodide (1 mmol) and 7-chloro-4-hydrazineylquinoline (1 mmol) were dissolved in 1 ml DMSO, 10 mg CH₃COOH was added and the mixture was heated at 100° C. for 30 min. Then mixture was cooled and 3 ml MeOH, stirred for 2 hours, filtered, and the solvent was evaporated. The residue was dissolved in the mixture of DMF/iPrOH (1/1), stirred for 2 h at 50° C., cooled, filtered, washed with iPrOH and dried. Yield: 32%.

¹H NMR (400 MHz, DMSO-d₆) δ 11.56-10.75 (m, 4H), 10.07 (s, 3H), 9.10 (s, 3H), 8.57 (dd, J=61.4, 21.8 Hz, 8H), 8.16-7.91 (m, 6H), 7.43 (s, 6H), 3.99 (s, 9H), 2.55 (d, J=13.5 Hz, 10H).

LCMS Rₜ=0.77 min using Method 1, MS ES-API calcd. for C₂₀H₁₈ClN₆ [M+H]⁺ 377.2, found 377.0.

### Example 24: Preparation of (E)-6-chloro-1-(2-(1-(6-(2,4-dimethyl-1H-imidazol-1-yl)pyridin-3-yl)ethylidene)hydrazineyl)phthalazine (24)

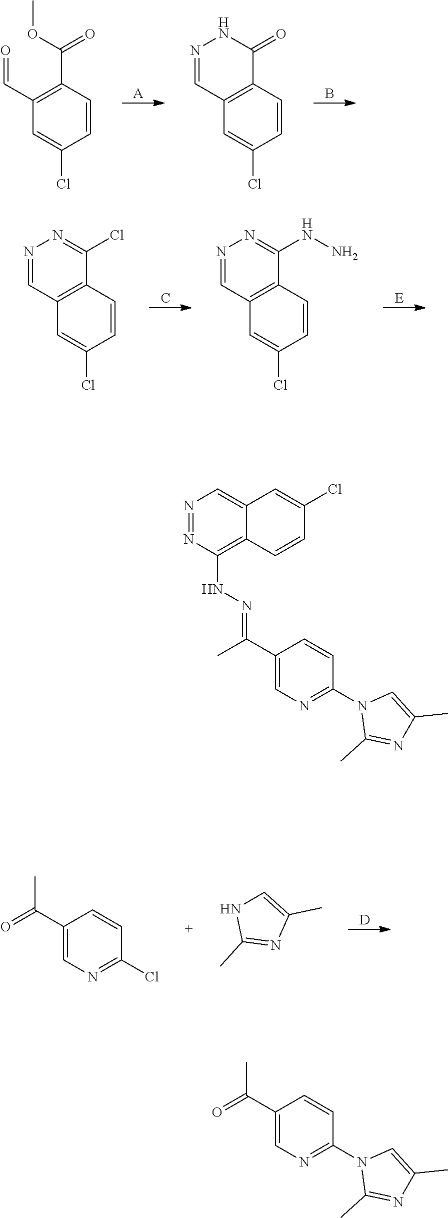

### Step A: Preparation of 6-chlorophthalazin-1(2H)-one

To a solution of compound methyl 4-chloro-2-formylbenzoate (4 mmol) in MeOH (40 ml) was added hydrazine hydrate (2.2 mmol) and the reaction mixture was refluxed for 2 h. The solvent was evaporated, and the residue was purified by HPLC. Yield: 83%.

### Step B: Preparation of 1,6-dichlorophthalazine

6-chlorophthalazin-1(2H)-one (4 mmol) was mixtures with phosphoryl trichloride (12 mmol) at 0° C. with stirring and NEt3 (1 mmol) was added. The mixture was stirred at room temperature for 1 h and then stirred at 50° C. for 12 h, cooled and then 50 g of ice was added. The precipitate that formed was filtered, washed with water and dried. Yield: 43%.

### Step C: Preparation of 6-chloro-1-hydrazineylphthalazine

To a suspension of compound 1,6-dichlorophthalazine (1 mmol) in anhydrous dioxane (10 ml) hydrazine hydrate (1.1 mmol) was added. The reaction mixture was refluxed for 24 h. The solvent was evaporated, and the residue was treated with 20 ml of iPrOH to give a yellow solid. The crude target compound was purified by HPLC. Yield: 42%.

### Step D: Preparation of 1-(6-(2,4-dimethyl-1H-imidazol-1-yl)pyridin-3-yl)ethan-1-one

To a solution of compound 1-(6-chloropyridin-3-yl)ethan-1-one (1 mmol) and 2,4-dimethyl-1H-imidazole (1 mmol) in dry pyridine (10 mL) was heated at 90° C. for 12-15 h with stirring. The reaction mixture was cooled, 50 ml of water was added, and the mixture was stirred for 1 h at r.t. The precipitate was filtered, washed with water (3×25 ml) and dried. The crude target compound was purified by recrystallization from a mixture of DMF/iPrOH (1/1). Yield: 48%.

### Step E: Preparation of (E)-6-chloro-1-(2-(1-(6-(2,4-dimethyl-1H-imidazol-1-yl)pyridin-3-yl)ethylidene)hydrazineyl)phthalazine

Preparation of 6-chloro-1-hydrazineylphthalazine (1 mmol) and 1-(6-(2,4-dimethyl-1H-imidazol-1-yl)pyridin-3-yl)ethan-1-one (1 mmol) were dissolved in 1 ml DMSO, 10 mg CH₃COOH was added and the mixture was heated at 100° C. for 30 min. Then mixture was cooled and 3 ml MeOH, stirred for 2 hours, filtered, evaporated. The residue was purified using HPLC Method A to provide the product as a solid. Yield: 82%.

¹H NMR (400 MHz, DMSO-d₆) δ 12.21 (s, 1H), 9.22 (s, 1H), 8.74 (d, J=6.6 Hz, 1H), 8.37 (d, J=8.6 Hz, 1H), 8.08 (s, 1H), 7.88 (s, 1H), 7.84-7.74 (m, 1H), 7.57 (d, J=8.6 Hz, 1H), 7.34 (s, 1H), 2.53 (d, J=10.3 Hz, 4H), 2.11 (s, 2H).

LCMS Rₜ=1.2 min using Method 1, MS ES-API calcd. for C₂₀H₁₈ClN₇ [M+H]⁺ 392, found 392.

### Example 25: Preparation of 7-chloro-N-(6-(4,5-dimethyl-1H-imidazol-1-yl)pyridin-3-yl)quinazolin-4-amine (25)

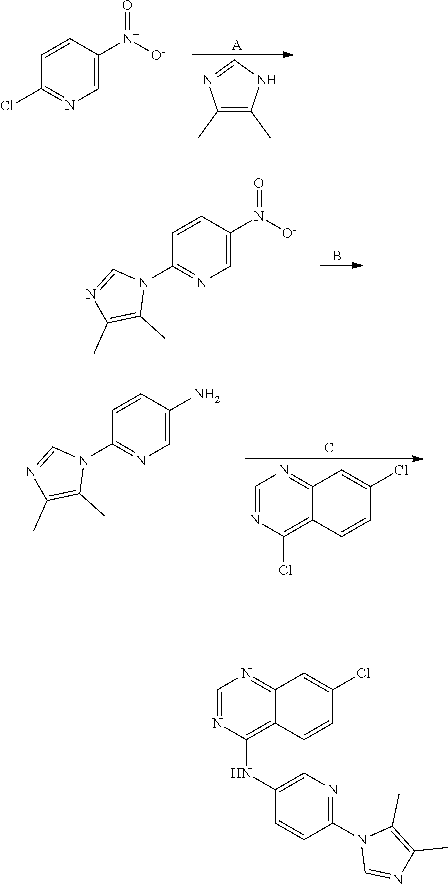

### Step A: Preparation of 2-(4,5-dimethyl-1H-imidazol-1-yl)-5-nitropyridine

To a solution of 2-chloro-5-nitropyridine (1 mmol) and 4,5-dimethyl-1H-imidazole (1.3 mmol) in dry DMSO (5 mL) as added K₂CO₃ (3 mmol) and heated at 90° C. for 5-7 days. After the completion, the reaction mixture was cooled, and 50 mL of water was added and then the mixture was stirred for 1 hour. The precipitate that formed was filtered and washed with water (3×25 mL). The crude target compound was purified by recrystallized from a mixture of DMF:iPrOH (1:5). Yield: 47%.

### Step B: Preparation of 6-(4,5-dimethyl-1H-imidazol-1-yl)pyridin-3-amine

To a solution of compound 2-(4,5-dimethyl-1H-imidazol-1-yl)-5-nitropyridine (1 mmol) in methanol (10 mL) was added 5% palladium on carbon (10 mg), and the mixture was stirred under a hydrogen atmosphere at room temperature for 10 h. The reaction mixture was filtered through Celite. The filtrate was concentrated under reduced pressure and purified by HPLC. Yield: 69%.

### Step C: Preparation of 7-chloro-N-(6-(4,5-dimethyl-1H-imidazol-1-yl)pyridin-3-yl)quinazolin-4-amine

Compound 6-(4,5-dimethyl-1H-imidazol-1-yl)pyridin-3-amine (1 mmol) and compound 4,7-dichloroquinazoline (1 mmol) were dissolved in 10 mL of dry acetic acid and the mixture was heated at 90° C. for 48 h. After the completion, the reaction mixture was cooled, filtered and solvent was evaporated. The residue was treatment with iPrOH (20 mL) and stirred for 30 min at room temperature. The precipitate was filtered, washed with iPrOH (3×20 mL) and dried. The crude target compound was purified by HPLC, Method A to provide a solid. Yield: 27%.

¹H NMR (400 MHz, DMSO-d₆) δ 8.67 (s, 1H), 8.44 (d, J=8.8 Hz, 1H), 8.32-8.22 (m, 2H), 7.78 (s, 1H), 7.55 (s, 1H), 7.40 (d, J=9.2 Hz, 2H), 3.13 (s, 1H), 2.20 (s, 3H), 2.07 (s, 3H).

LCMS Rₜ=1.0 min using Method 1, MS ES-API calcd. for C₁₈H₁₅ClN₆ [M+H]⁺ 351.0, found 351.0.

### Example 26: Preparation of (E)-1-(5-(1-(2-(7-fluoroquinazolin-4-yl)hydrazineylidene)ethyl)pyridin-2-yl)-3-methyl-1H-imidazol-3-ium phosphoric acid salt (26)

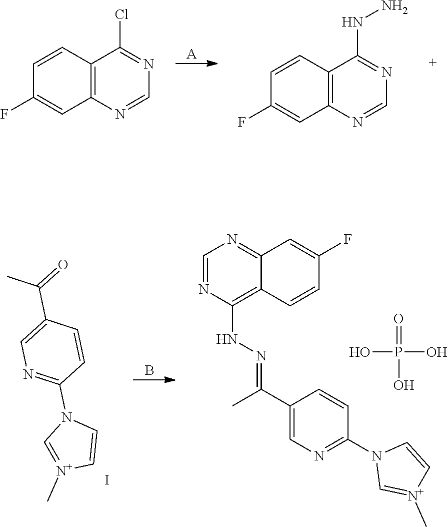

### Step A: Preparation of 7-fluoro-4-hydrazineylquinazoline

To a suspension of compound 4-chloro-7-fluoroquinazoline (2 mmol) in anhydrous THE (10 ml) hydrazine hydrate (2.2 mmol) was added. The reaction mixture was stirred at room temperature for 18 h. The solvent was evaporated at r.t. until a precipitate was formed. The precipitate was filtered and washed with cold THE to give a yellow solid. The crude target compound was purified by LC. Yield: 65%.

### Step B: Preparation of (E)-1-(5-(1-(2-(7-fluoroquinazolin-4-yl)hydrazineylidene)ethyl)pyridin-2-yl)-3-methyl-1H-imidazol-3-ium phosphoric acid salt

7-fluoro-4-Hydrazineylquinazoline (1 mmol) and 1-(5-acetylpyridin-2-yl)-3-methyl-1H-imidazol-3-ium iodide (1 mmol) prepared as described in Example 17 were dissolved in 1 ml DMSO then 10 mg CH₃COOH was added and the mixture was heated at 100° C. for 30 min. Then mixture was cooled and 3 ml/1 ml MeOH/H₃PO₄ was added, stirred for 2 hours, filtered the precipitate, evaporated solvent. The residue was treatment with iPrOH (20 mL) and stirred for 30 min at room temperature. The precipitate was filtered, washed with iPrOH (3×20 mL) and dried. Yield: 39%.

¹H NMR (400 MHz, DMSO-d₆) δ 11.79 (s, 1H), 10.07 (s, 1H), 9.31 (s, 1H), 8.83 (d, J=8.5 Hz, 1H), 8.56 (s, 1H), 8.43-8.24 (m, 1H), 8.02 (dd, J=38.3, 13.6 Hz, 3H), 7.32 (dd, J=13.8, 9.3 Hz, 2H), 4.00 (s, 3H), 2.71-2.55 (m, 3H).

LCMS Rₜ=0.97 min using Method 1, MS ES-API calcd. for C₁₉H₁₈FN₇ [M+H]⁺ 363.2, found 363.1.

### Example 27: Preparation of (E)-7-chloro-4-(2-(1-(6-(2,4-dimethyl-1H-imidazol-1-yl)pyridin-3-yl)ethylidene)hydrazineyl)quinazoline HCL salt (27)

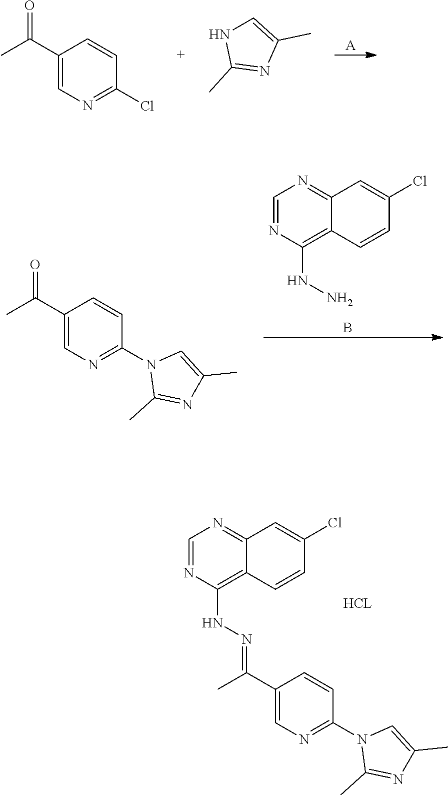

### Step A: Preparation of 1-(6-(2,4-dimethyl-1H-imidazol-1-yl)pyridin-3-yl)ethan-1-one

To a solution of compound 1 (1 mmol) and 2 (1.1 mmol) in dry pyridine (10 mL) was heated at 90° C. for 12-15 h with stirring. The reaction mixture was cooled, 50 ml of water was added, and the mixture was stirred for 1 h at r.t. The precipitate that formed was filtered, washed with water (3×25 ml) and dried. The crude target compound was purified by recrystallization from mixture of DMF/iPrOH (1/1). Yield: 48%.

### Step B: Preparation of (E)-7-chloro-4-(2-(1-(6-(2,4-dimethyl-1H-imidazol-1-yl)pyridin-3-yl)ethylidene)hydrazineyl)quinazoline HCL salt

1-(6-(2,4-dimethyl-1H-imidazol-1-yl)pyridin-3-yl)ethan-1-one (1 mmol), 7-chloro-4-hydrazineylquinazoline (prepared as in Example 15, 1 mmol) were dissolved in 1 ml DMSO, 10 mg CH₃COOH was added, heated at 100° C. for 30 min. Then mixture was cooled and 3 ml/1 ml MeOH/HCl, stirred for 2 hours, filtered, evaporated. The crude target compound was purified by recrystallized from a mixture of DMF:iPrOH (1:5). Yield: 48%.

¹H NMR (400 MHz, DMSO-d₆) δ 11.81 (s, 1H), 9.25 (s, 1H), 8.74 (d, J=6.6 Hz, 1H), 8.62 (d, J=8.6 Hz, 1H), 8.08 (s, 1H), 7.88 (s, 1H), 7.84-7.74 (m, 1H), 7.57 (d, J=8.6 Hz, 1H), 7.34 (s, 1H), 2.53 (d, J=10.3 Hz, 4H), 2.11 (s, 2H).

### Example 28: Preparation of 7-chloro-4-(2-(1-(2-(4,5-dimethyl-1H-imidazol-1-yl)pyridin-4-yl)ethylidene)hydrazineyl)quinazoline (28)

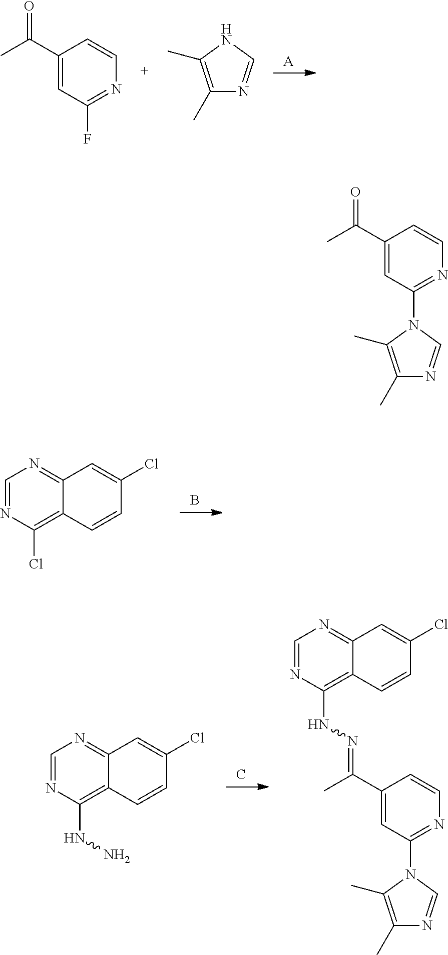

### Step A: Preparation of 1-(2-(4,5-dimethyl-1H-imidazol-1-yl)pyridin-4-yl)ethan-1-one

A solution of 1-(2-fluoropyridin-4-yl)ethan-1-one (1 mmol) and 4,5-dimethyl-1H-imidazole (1.1 mmol) in dry pyridine (10 mL) was heated at 90° C. for 12-15 h with stirring. The reaction mixture was cooled, and 50 ml of water was added, and the mixture was stirred for 1 h at r.t. The precipitate the formed was filtered, washed with water (3×25 ml) and dried. The crude target compound was purified by recrystallization from a mixture of DMF/iPrOH (1/1). Yield: 54%.

### Step B: Preparation of 7-chloro-4-hydrazineylquinazoline

To a suspension of 4,7-dichloroquinazoline (2 mmol) in anhydrous THE (10 ml) was added hydrazine hydrate (2.2 mmol). The reaction mixture was stirred at room temperature for 18 h. The solvent was evaporated at r.t. until a precipitate formed. The precipitate was filtered and washed with cold THE to give a yellow solid. The crude target compound was purified by HPLC. Yield: 67%.

### Step C: Preparation of 7-chloro-4-(2-(1-(2-(4,5-dimethyl-1H-imidazol-1-yl)pyridin-4-yl)ethylidene)hydrazineyl)quinazoline

To a solution of 7-chloro-4-hydrazineylquinazoline (1 mmol) and 1-(2-(4,5-dimethyl-1H-imidazol-1-yl)pyridin-4-yl)ethan-1-one (1 mmol) in 1 ml DMSO was added 10 mg CH₃COOH and the reaction mixture was heated at 100° C. for 30 min. Then mixture was cooled and 3 ml MeOH was added and the mixture was stirred for 2 hours. The mixture was filtered and the solvent was evaporated. The residue was purified using HPLC Method A to provide the product as a solid. Yield: 46%.

¹H NMR (400 MHz, DMSO-d₆) δ 11.79 (s, 1H), 8.63 (d, J=5.1 Hz, 1H), 8.31 (d, J=8.5 Hz, 1H), 8.19 (s, 1H), 8.08-7.91 (m, 3H), 7.63-7.44 (m, 2H), 2.57 (s, 3H), 2.28 (d, J=12.4 Hz, 3H), 2.12 (d, J=14.6 Hz, 3H).

LCMS Rₜ=3.0 min using Method 1, MS ES-API calcd. for C₂₀H₁₈ClN₇ [M+H]⁺ 392.2, found 392.2.

### Example 29: Preparation of 1-(3-(1-(2-(7-chloroquinazolin-4-yl)hydrazineylidene)ethyl)phenyl)—N,N-dimethylmethanamine (29)

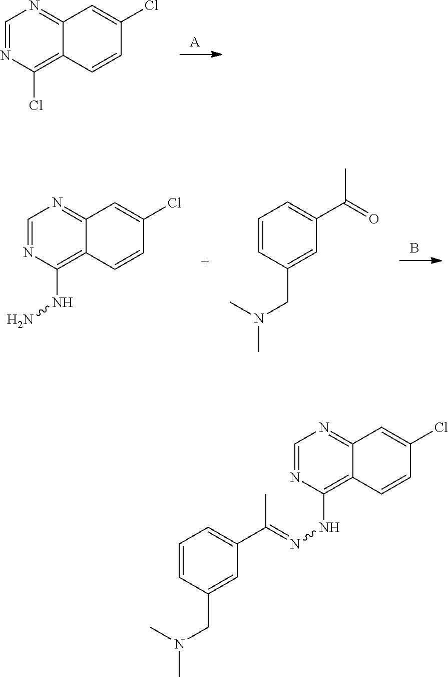

### Step A: Preparation of 7-chloro-4-hydrazineylquinazoline

To a suspension of 4,7-dichloroquinazoline (2 mmol) in anhydrous THE (10 ml) hydrazine hydrate (2.2 mmol) was added. The reaction mixture was stirred at room temperature for 18 h. The solvent was partially evaporated at r.t. until a precipitate was formed. The precipitate was filtered and washed with cold THE to give a yellow solid. The crude target compound was purified by HPLC. Yield: 67%.

### Step B: Preparation of 1-(3-(1-(2-(7-chloroquinazolin-4-yl)hydrazineylidene)ethyl)phenyl)—N,N-dimethylmethanamine

7-Chloro-4-hydrazineylquinazoline (1 mmol) and 1-(3-((dimethylamino)methyl)phenyl)ethan-1-one (1 mmol) were dissolved in 1 ml DMSO then 10 mg CH₃COOH was added and the mixture was heated at 100° C. for 30 min. Then mixture was cooled and 3 ml MeOH, stirred for 2 hours, filtered, evaporated. The residue was purified using HPLC Method A to provide the product as a solid. Yield: 42%.

¹H NMR (400 MHz, DMSO-d₆) δ 11.46 (s, 1H), 8.24 (d, J=8.5 Hz, 1H), 8.01 (d, J=7.2 Hz, 1H), 7.87 (d, J=9.8 Hz, 2H), 7.41 (ddd, J=18.0, 16.5, 10.6 Hz, 4H), 3.45 (s, 2H), 2.17 (s, 6H).

LCMS Rₜ=2.7 min using Method 1, MS ES-API calcd. for C₁₉H₂₀ClN₅ [M+H]⁺ 354.2, found 354.4.

### Example 30: Preparation of 7-chloro-4-(2-(1-(3-(4-methylpiperazin-1-yl)phenyl)ethylidene)hydrazineyl)quinazoline (30)

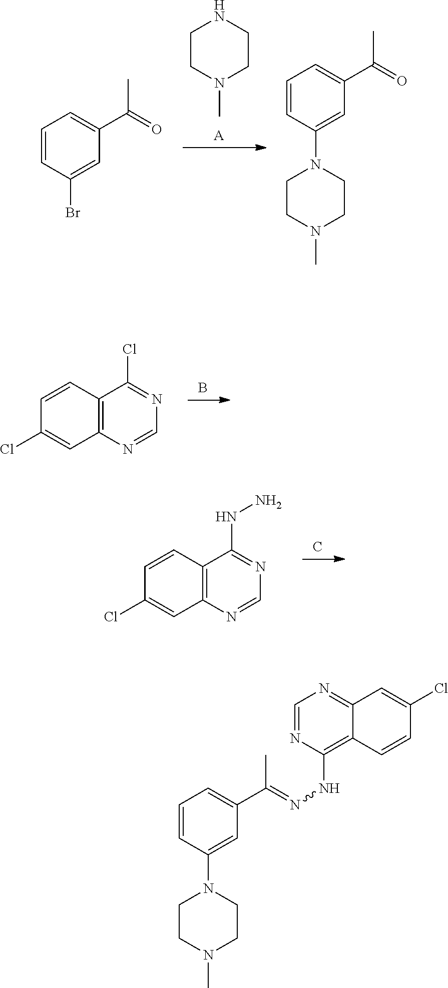

### Step A: Preparation of 1-(3-(4-methylpiperazin-1-yl)phenyl)ethan-1-one

1-methylpiperazine (1 mmol), DIPEA (1.3 eq) and 1-(3-bromophenyl)ethan-1-one (1 mmol) were placed in a vial and dissolved in dry DMSO (0.35 mL). The reaction mixture was left at room temperature for 1 h. Then the reaction mixture was heated with stirring at 100° C. for 12 h. After cooling to the ambient temperature, the solvent was evaporated. The residue was dissolved in DMSO and filtered. The solution was subjected to HPLC purification. Yield: 74%.

### Step B: Preparation of 7-chloro-4-hydrazineylquinazoline

To a suspension of 4,7-dichloroquinazoline (2 mmol) in anhydrous THE (10 ml) hydrazine hydrate (2.2 mmol) was added. The reaction mixture was stirred at room temperature for 18 h. The solvent was partially evaporated at r.t. until a precipitate was formed. The precipitate was filtered and washed with cold THE to give a yellow solid. The crude target compound was purified by HPLC. Yield: 51%.

### Step C: Preparation of 7-chloro-4-(2-(1-(3-(4-methylpiperazin-1-yl)phenyl)ethylidene)hydrazineyl)quinazoline

7-Chloro-4-hydrazineylquinazoline (1 mmol) and 1-(3-(4-methylpiperazin-1-yl)phenyl)ethan-1-one (1 mmol) were dissolved in 1 ml DMSO and 10 mg CH₃COOH was added, and then the mixture was heated at 100° C. for 30 min. Then mixture was cooled and 3 ml MeOH, stirred for 2 hours, filtered, evaporated. The residue was purified using HPLC, Method A to provide the product as a solid. Yield: 38%.

¹H NMR (400 MHz, DMSO-d₆) δ 11.44 (s, 1H), 8.23 (d, J=8.5 Hz, 1H), 7.84 (s, 1H), 7.62-7.38 (m, 4H), 7.27 (t, J=7.9 Hz, 1H), 7.00 (d, J=8.0 Hz, 1H), 3.33 (s, 3H), 2.23 (s, 3H).

LCMS Rₜ=1.11 min using Method 1, MS ES-API calcd. for C₂₁H₂₃ClN₆ [M+H]⁺ 395.2, found 395.2.

### Example 31: Preparation of 6-chloro-N-(6-(4,5-dimethyl-1H-imidazol-1-yl)-5-methoxypyridin-3-yl)phthalazin-1-amine phosphoric acid salt (31)

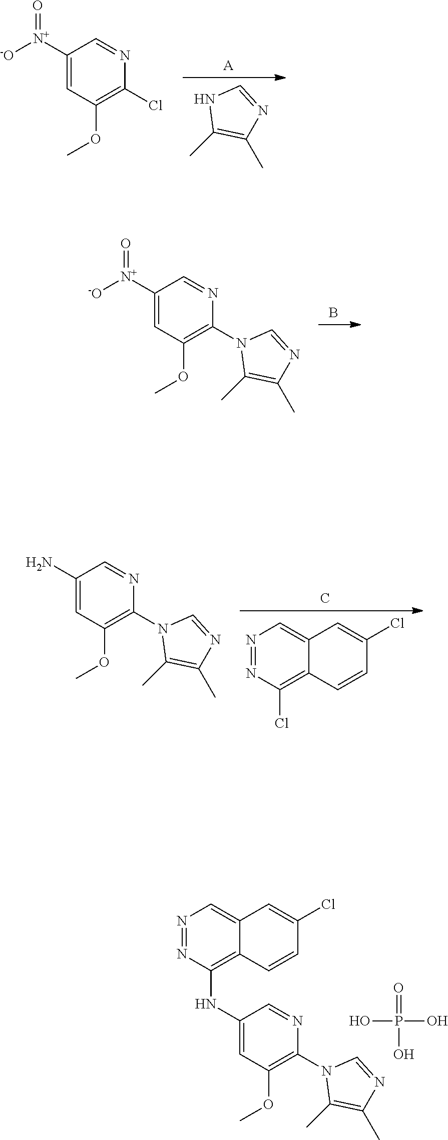

### Step A: Preparation of 2-(4,5-dimethyl-1H-imidazol-1-yl)-3-methoxy-5-nitropyridine

2-Chloro-3-methoxy-5-nitropyridine (5 mmol) and 4,5-dimethyl-1H-imidazole (6 mmol) were dissolved in 20 ml of dry DMSO. K₂CO₃ (12 mmol) was added to the solution and the mixture was heated at 90° C. for 10 days. Then the mixture was cooled, and 50 ml of water was added then the mixture was stirred for 1 hour. The solid that formed was filtered and washed with water (3×50 ml). The crude target compound was purified by recrystallization from mixture of DMF/iPrOH (1/5). Yield: 45%.

### Step B: Preparation of 6-(4,5-dimethyl-1H-imidazol-1-yl)-5-methoxypyridin-3-amine

To a solution of 2-(4,5-dimethyl-1H-imidazol-1-yl)-3-methoxy-5-nitropyridine (2 mmol) in methanol (12 mL) was added 5% palladium carbon (20 mg), and the mixture was stirred under a hydrogen atmosphere at room temperature for 18 h. The reaction mixture was filtered through Celite. The filtrate was concentrated under reduced pressure and after was purified by using HPLC. Yield: 58%.

### Step C: Preparation of 6-chloro-N-(6-(4,5-dimethyl-1H-imidazol-1-yl)-5-methoxypyridin-3-yl)phthalazin-1-amine phosphoric acid salt

6-(4,5-Dimethyl-1H-imidazol-1-yl)-5-methoxypyridin-3-amine (1 mmol) and 1,6-dichlorophthalazine (1 mmol) were dissolved in 12 ml of dry HOAc and the mixture was heated at 90° C. for 56 h with stirring. Then the reaction mixture was cooled, filtered and solvent was evaporated. The residue was treatment with iPrOH (20 ml) and stirred for 30 min at room temperature. The precipitate that formed was filtered, washed with iPrOH (3×20 ml) and dried. The crude target compound was purified by HPLC. Then mixture was cooled and 3 ml/1 ml MeOH/H₃PO₄ was added, and the mixture was stirred for 2 hours. The precipitate was isolated by filtration. Yield: 37%.

¹H NMR (400 MHz, DMSO-d₆) δ 9.81-9.56 (m, 6H), 9.21 (s, 5H), 8.64 (s, 9H), 8.48-8.02 (m, 16H), 7.66 (s, 6H), 3.85 (s, 18H), 2.11 (s, 13H), 2.01 (s, 14H).

### Example 32: Preparation of (E)-4-(2-(1-(6-(1H-1,2,4-triazol-1-yl)pyridin-3-yl)ethylidene)hydrazineyl)-7-chloroquinazoline (32)

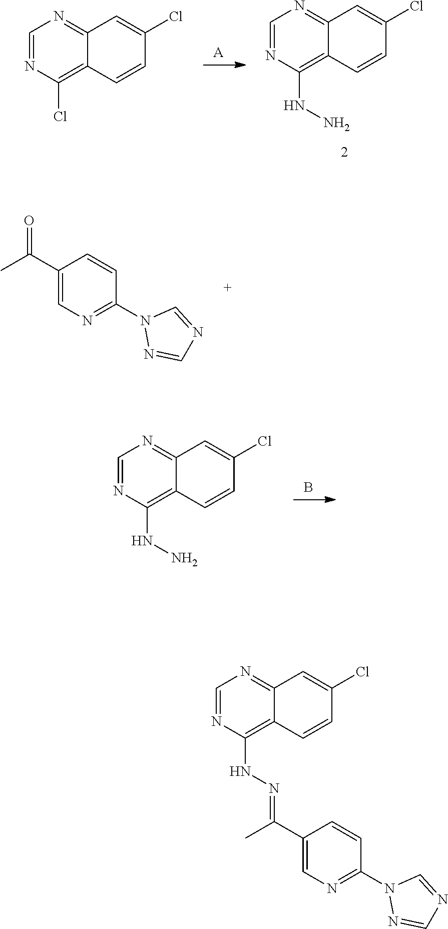

### Step A: 7-chloro-4-hydrazineylquinazoline

To a suspension of 4,7-dichloroquinazoline (1 mmol) in anhydrous THE (10 ml) hydrazine hydrate (1.8 mmol) was added. The reaction mixture was stirred at room temperature for 18 h. The solvent was partially evaporated at r.t. until a precipitate formed. The precipitate was filtered and washed with cold THE to give a yellow solid. The crude target compound was purified by HPLC. Yield: 57%.

### Step B: Preparation of (E)-4-(2-(1-(6-(1H-1,2,4-triazol-1-yl)pyridin-3-yl)ethylidene)hydrazineyl)-7-chloroquinazoline

1-(6-(1H-1,2,4-Triazol-1-yl)pyridin-3-yl)ethan-1-one (1 mmol) and 7-chloro-4-hydrazineylquinazoline (1 mmol) were dissolved in 1 ml DMSO and 10 mg CH₃COOH was added and them the reaction mixture was heated at 100° C. for 50 min. Then mixture was cooled and 3 ml MeOH, 0.2 g of C-18 chromatographic phase and HCl conc. (3 mmol—solution in dioxane) were added, stirred for 2 hours, filtered, evaporated. The residue was purified using HPLC Method A to provide the product as a solid. Yield: 34%.

¹H NMR (400 MHz, DMSO-d₆) δ 9.29 (s, 1H), 8.29 (dd, J=36.0, 22.9 Hz, 4H), 7.82 (t, J=10.9 Hz, 2H), 7.66 (d, J=8.2 Hz, 1H), 7.02 (s, 1H), 6.89 (d, J=6.5 Hz, 1H), 2.23 (s, 3H).

LCMS Rₜ=1.07 min using Method 1, MS ES-API calcd. for C₁₈H₁₄ClN₇ [M+H]⁺ 365.1, found 365.2.

### Example 33: Preparation of 7-chloro-N-(6-(4,5-dimethyl-1H-imidazol-1-yl)-5-methoxypyridin-3-yl)isoquinolin-4-amine phosphoric acid salt (33)

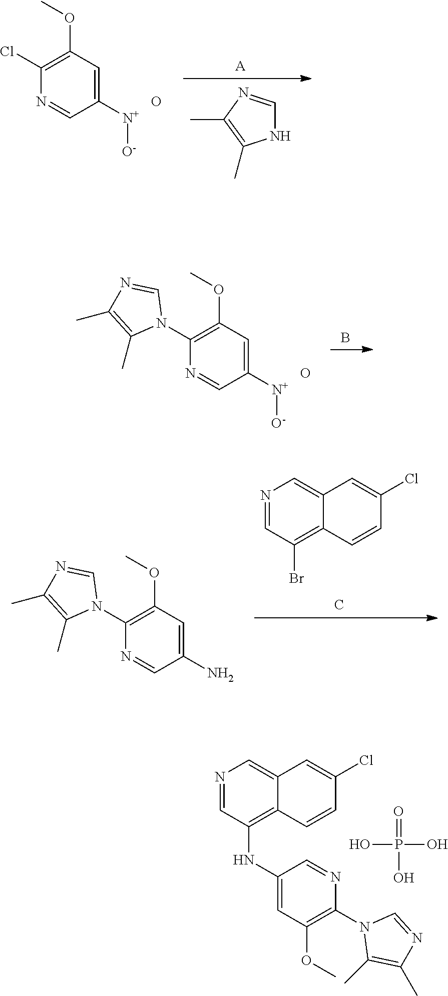

### Step A: Preparation of 2-(4,5-dimethyl-1H-imidazol-1-yl)-3-methoxy-5-nitropyridine

2-Chloro-3-methoxy-5-nitropyridine (4 mmol) and 4,5-dimethyl-1H-imidazole (5 mmol) were dissolved in 15 ml of dry DMSO, K₂CO₃ (12 mmol) was added, heated at 90° C. for 7 days. Then mixture was cooled, and 50 ml of water was added. The mixture was stirred for 1 hour and the precipitate that formed was filtered and washed with water (3×50 ml). The crude target compound was purified by recrystallization from a mixture of DMF/iPrOH (1/5). Yield: 61%.

### Step B: Preparation of 6-(4,5-dimethyl-1H-imidazol-1-yl)-5-methoxypyridin-3-amine

To a solution of 2-(4,5-dimethyl-1H-imidazol-1-yl)-3-methoxy-5-nitropyridine (2 mmol) in methanol (10 mL) was added 5% palladium carbon (10 mg), and the mixture was stirred under a hydrogen atmosphere at room temperature for 15 h. The reaction mixture was filtered through Celite. The filtrate was concentrated under reduced pressure and after was purified by using HPLC.

Yield: 42%.

### Step C: Preparation of 7-chloro-N-(6-(4,5-dimethyl-1H-imidazol-1-yl)-5-methoxypyridin-3-yl)isoquinolin-4-amine phosphoric acid salt

6-(4,5-Dimethyl-1H-imidazol-1-yl)-5-methoxypyridin-3-amine (1 mmol) and 4-bromo-7-chloroisoquinoline (1 mmol) were dissolved in 10 ml of dry HOAc and the mixture was heated at 90° C. for 48 h with stirring. Then the reaction mixture was cooled, filtered and solvent was evaporated. The residue was treatment with iPrOH (20 ml) and stirred for 30 min at room temperature. The precipitate was filtered, washed with iPrOH (3×20 ml) and dried. The crude target compound was purified by HPLC. Then mixture was cooled and 3 ml/1 ml MeOH/H₃PO₄ was added and after stirring for 2 hours the precipitate that was formed was filtered to provide the product as a solid. Yield: 25%.

¹H NMR (400 MHz, DMSO-d₆) δ 9.02 (s, 1H), 8.85 (s, 1H), 8.55 (s, 1H), 8.29 (s, 1H), 8.17 (d, J=9.1 Hz, 1H), 7.84 (d, J=5.7 Hz, 2H), 7.69 (s, 1H), 7.26 (s, 1H), 3.72 (s, 3H), 2.08 (s, 3H), 1.96 (s, 3H).

LCMS Rₜ=0.85 min using Method 1, MS ES-API calcd. for C₂₀H₁₈ClN₅O [M+H]⁺ 380.1, found 380.0.

### Example 34: Preparation of N-(6-(1H-1,2,4-triazol-1-yl)pyridin-3-yl)-7-chloroquinazolin-4-amine phosphoric acid salt (34)

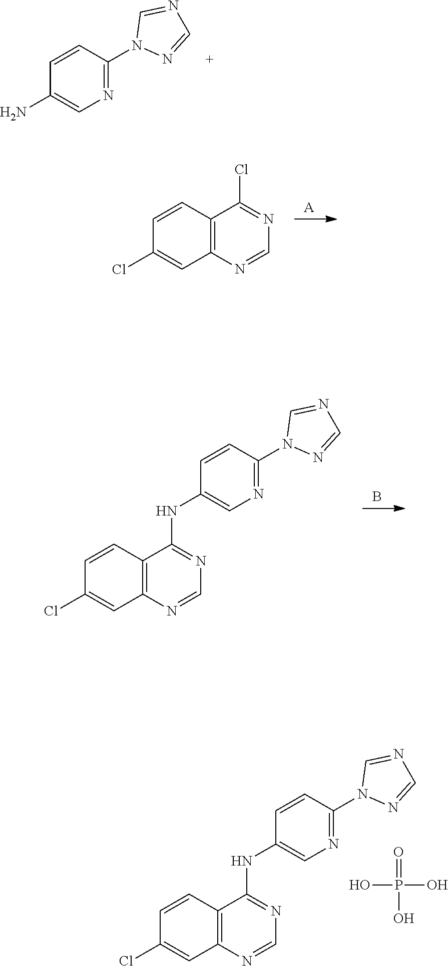

### Step A: Preparation of N-(6-(1H-1,2,4-triazol-1-yl)pyridin-3-yl)-7-chloroquinazolin-4-amine

N-(6-(1H-1,2,4-Triazol-1-yl)pyridin-3-yl)-7-chloroquinazolin-4-amine (5 mmol) and 4,7-dichloroquinazoline (5 mmol) were dissolved in 15 ml of dry HOAc and the mixture was heated at 90° C. for 48 h with stirring. Then the reaction mixture was cooled, filtered and solvent was evaporated. The residue was treatment with iPrOH (20 ml) and stirred for 30 min at room temperature. The precipitate was filtered, washed with iPrOH (3×20 ml) and dried. Yield: 81%.

### Step B: N-(6-(1H-1,2,4-triazol-1-yl)pyridin-3-yl)-7-chloroquinazolin-4-amine phosphoric acid salt

N-(6-(1H-1,2,4-triazol-1-yl)pyridin-3-yl)-7-chloroquinazolin-4-amine (1 mmol) was dissolved in 3 ml/1 ml MeOH/H₃PO₄ then mixture was cooled and stirred for 2 hours, filtered. Yield: 87%.

¹H NMR (400 MHz, DMSO-d₆+CCl₄) δ 10.11 (s, 1H), 9.14 (s, 1H), 8.99 (s, 1H), 8.60 (t, J=10.9 Hz, 3H), 8.06 (s, 1H), 7.95-7.43 (m, 4H).

LCMS Rₜ=2.3 min using Method 1, MS ES-API calcd. for C₁₅H₁₀ClN₇ [M+H]⁺ 324.1, found 324.2.

### Example 35: Preparation of (E)-7-chloro-4-(2-(3-fluoro-4-(1H-imidazol-1-yl)benzylidene)hydrazineyl)quinoline HCL Salt

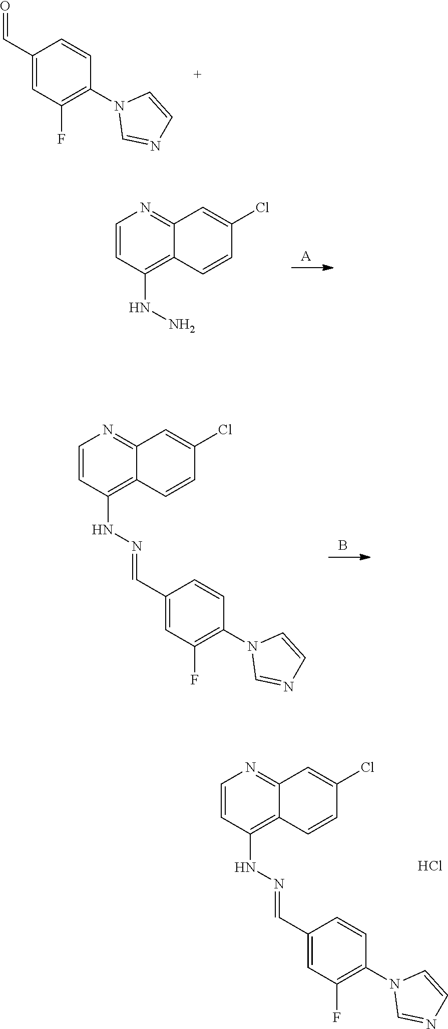

### Step A: Preparation of (E)-7-chloro-4-(2-(3-fluoro-4-(1H-imidazol-1-yl)benzylidene)hydrazineyl)quinoline

3-Fluoro-4-(1H-imidazol-1-yl)benzaldehyde (4 mmol), 7-chloro-4-hydrazineylquinoline (prepared as described in Example 15, 4 mmol) were dissolved in 2 ml DMSO, 10 mg CH₃COOH was added, heated at 100° C. for 50 min. Then mixture was cooled and 3 ml MeOH, 0.2 g of C-18 chromatographic phase and HCl conc. (3 mmol—solution in dioxane) were added, stirred for 2 hours, filtered, evaporated. The crude target compound was purified by HPLC Method A. Yield: 36%.

### Step B: Preparation of (E)-7-chloro-4-(2-(3-fluoro-4-(1H-imidazol-1-yl)benzylidene)hydrazineyl)quinoline HCL Salt

(E)-7-Chloro-4-(2-(3-fluoro-4-(1H-imidazol-1-yl)benzylidene)hydrazineyl)quinoline (1 mmol) was dissolved in 3 ml/1 ml MeOH/HCL. The mixture was stirred for 2 hours, evaporated and the residue was purified using LC. Yield: 89%.

¹H NMR (400 MHz, DMSO-d₆) δ 11.45 (s, 1H), 8.44 (dd, J=51.7, 42.5 Hz, 3H), 8.11 (s, 1H), 7.68 (ddd, J=226.9, 102.2, 69.4 Hz, 7H).

LCMS Rₜ=0.84 min using Method 1, MS ES-API calcd. for C₁₉H₁₃ClFN₅ [M+H]⁺ 366.1, found 366.0.

### Example 36: Preparation of 7-chloro-N-(6-(2,4-dimethyl-1H-imidazol-1-yl)pyridin-3-yl)isoquinolin-4-amine phosphoric acid salt (37)

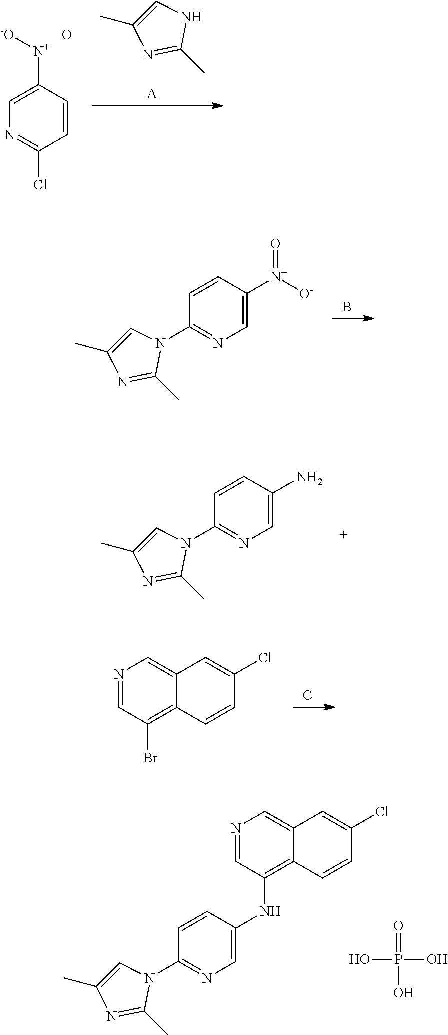

### Step A: Preparation of 2-(2,4-dimethyl-1H-imidazol-1-yl)-5-nitropyridine

2-Chloro-5-nitropyridine (1 mmol) and 2,4-dimethyl-1H-imidazole (1.3 mmol) were dissolved in 5 ml of dry DMSO and K₂CO₃ (3 mmol) was added. Then the mixture was heated at 90° C. for 7 days. The mixture was cooled and then 25 ml of water was added, and the mixture was stirred for 1 hour. The precipitate the formed was filtered and washed with water (3×25 ml). The crude target compound was purified by recrystallization from a mixture of DMF/iPrOH (1/5). Yield: 64%.

### Step B: Preparation of 6-(2,4-dimethyl-1H-imidazol-1-yl)pyridin-3-amine

To a solution of 2-(2,4-dimethyl-1H-imidazol-1-yl)-5-nitropyridine (1 mmol) in methanol (10 mL) was added 5% palladium on carbon (10 mg), and the mixture was stirred under a hydrogen atmosphere at room temperature for 10 h. The reaction mixture was filtered through Celite. The filtrate was concentrated under reduced pressure and was purified by using LC. Yield: 69%.

### Step C: 7-chloro-N-(6-(2,4-dimethyl-1H-imidazol-1-yl)pyridin-3-yl)isoquinolin-4-amine phosphoric acid salt

6-(2,4-dimethyl-1H-imidazol-1-yl)pyridin-3-amine (1 mmol), K₂CO₃ (2 mmol) and 4-bromo-7-chloroisoquinoline (1 mmol) were dissolved in 10 ml of dry dioxane, Pddppf (5 mol %) was added and the mixture was heated at 90° C. for 48 h with stirring. Then the reaction mixture was cooled, filtered and the solvent was evaporated. The residue was treated with iPrOH (20 ml) and stirred for 30 min at room temperature. The precipitate was filtered, washed with iPrOH (3×20 ml) and dried. Then mixture was cooled and 3 ml MeOH, 0.2 g of C-18 chromatographic phase and H₃PO₄ (2 mmol) were added, stirred for 2 hours, filtered, evaporated. The residue was purified by HPLC, Method A. Yield: 27%.

LCMS Rₜ=2.6 min using Method 1, MS ES-API calcd. for C₁₉H₁₅N₅Cl [M+H]⁺ 350.1, found 350.2.

### Example 37: 5-((7-chloroquinazolin-4-yl)amino)-2-(4,5-dimethyl-1H-imidazol-1-yl)pyridin-3-ol phosphoric acid salt (40)

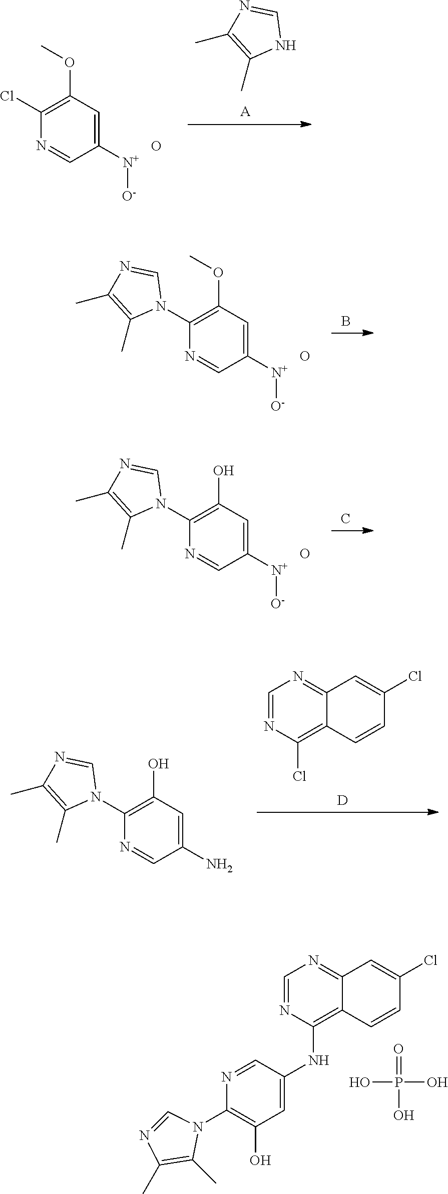

### Step A: Preparation of 2-(4,5-dimethyl-1H-imidazol-1-yl)-3-methoxy-5-nitropyridine

4,5-Dimethyl-1H-imidazole (5 mmol) and 2-chloro-3-methoxy-5-nitropyridine (6 mmol) were dissolved in 20 ml of dry DMSO and K₂CO₃ (12 mmol) was added and then the mixture was heated at 90° C. for 10 days. Then the mixture was cooled, and 50 ml of water was added, and the mixture was stirred for an hour. The residue was filtered and washed with water (3×50 ml). The crude target compound was purified by recrystallization from mixture of DMF/iPrOH (1/5). Yield: 45%.

### Step B: Preparation of 2-(4,5-dimethyl-1H-imidazol-1-yl)-5-nitropyridin-3-ol

2-(4,5-dimethyl-1H-imidazol-1-yl)-3-methoxy-5-nitropyridine (5 mmol) was dissolved in 20 ml of CH₂C₁₂, BBr₃ (20 mmol) was added, the mixture was heated at 40° C. for 48 h. Then the mixture was cooled, the solvent was evaporated, and the residue was purified by using HPLC Yield: 55%.

### Step C: Preparation of 5-amino-2-(4,5-dimethyl-1H-imidazol-1-yl)pyridin-3-ol

To a solution of 2-(4,5-dimethyl-1H-imidazol-1-yl)-5-nitropyridin-3-ol (2 mmol) in methanol (12 mL) was added 5% palladium carbon (20 mg), and the mixture was stirred under a hydrogen atmosphere at room temperature for 18 h. The reaction mixture was filtered through Celite. The filtrate was concentrated under reduced pressure and after resulting precipitate was purified by using HPLC. Yield: 58%.

### Step D: 5-((7-chloroquinazolin-4-yl)amino)-2-(4,5-dimethyl-1H-imidazol-1-yl)pyridin-3-ol phosphoric acid salt

5-Amino-2-(4,5-dimethyl-1H-imidazol-1-yl)pyridin-3-ol (1 mmol) and 4,7-dichloroquinazoline (1 mmol) were dissolved in 12 ml of dry HOAc and the mixture was heated at 90° C. for 56 h with stirring. Then the reaction mixture was cooled, filtered and solvent was evaporated. The residue was treated with iPrOH (20 ml) and stirred for 30 min at room temperature. The precipitate was filtered, washed with iPrOH (3×20 ml) and dried. The crude target compound was purified by HPLC Method A. Then mixture was cooled and 3 ml/1 ml MeOH/H₃PO₄, was stirred for 2 hours. The resulting precipitate was filtered. Yield: 37%.

¹H NMR (400 MHz, DMSO-d₆) δ 10.12 (s, 1H), 8.75-8.54 (m, 2H), 8.48 (s, 1H), 8.19 (s, 1H), 7.89 (d, J=1.9 Hz, 1H), 7.77 (d, J=8.9 Hz, 1H), 7.60 (s, 1H), 2.07 (d, J=22.4 Hz, 6H).

LCMS Rₜ=0.92 min using Method 1, MS ES-API calcd. for C₁₈H₁₅ClN₆O [M+H]⁺ 367.1, found 367.2.

### Example 38: Preparation of N-(6-(1H-imidazol-1-yl)pyridin-3-yl)-7-chloroquinazolin-4-amine (41)

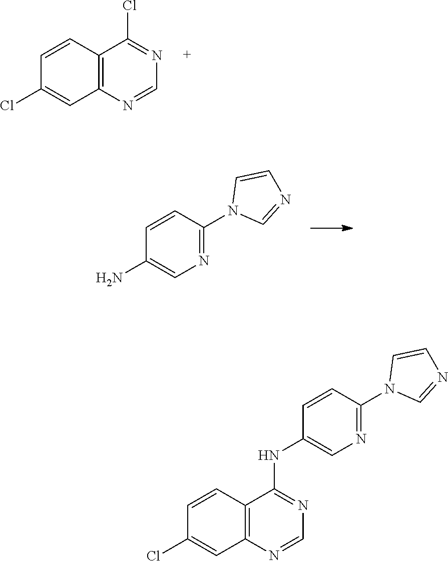 [SMILES: Clc1ccc2c(Cl)ncnc2c1 ; Nc1ccc(-n2ccnc2)nc1 ; Clc1ccc2c(Nc3ccc(-n4ccnc4)nc3)ncnc2c1]

A solution of 4,7-dichloroquinazoline (1 mmol) and 6-(1H-imidazol-1-yl)pyridin-3-amine (1.1 mmol) in dry pyridine (10 mL) was heated at 90° C. for 15 h. After the completion, the reaction mixture was cooled, 50 mL of water was added, and the mixture was stirred for 1 h at room temperature. The precipitate was filtered, washed with water (3×25 mL) and dried. The crude target compound was purified by recrystallization from 1:1 mixture of DMF:iPrOH. Yield: 33%.

¹H NMR (400 MHz, DMSO-d₆) δ 12.30 (s, 1H), 10.01 (s, 1H), 9.20 (d, J=9.0 Hz, 1H), 9.10 (d, J=2.5 Hz, 1H), 8.98 (s, 1H), 8.62 (dd, J=8.8, 2.6 Hz, 1H), 8.50 (s, 1H), 8.20 (d, J=8.8 Hz, 1H), 8.10 (d, J=2.2 Hz, 1H), 7.95 (s, 1H), 7.93 (d, J=2.2 Hz, 1H), 1.91 (s, 1H).

LCMS Rₜ=0.81 min using Method 1, MS ES-API calcd. for C₁₆H₁₁N₆Cl [M+H]⁺ 323.1, found 323.0.

### Example 39: Preparation of 7-chloro-N-(3-((diethylamino)methyl)phenyl)quinolin-4-amine (42)

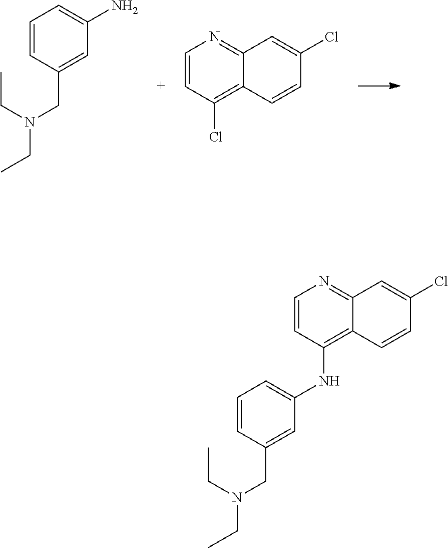 [SMILES: CCN(CC)Cc1cccc(N)c1 ; Clc1ccc2c(Cl)ccnc2c1 ; CCN(CC)Cc1cccc(Nc2ccnc3cc(Cl)ccc23)c1]

3-((diethylamino)methyl)aniline (1 mmol) and 4,7-dichloroquinoline (1 mmol) were dissolved in 10 mL of dry HOAc and the mixture was heated at 90° C. for 48 h. The reaction mixture was cooled, filtered and the solvent was evaporated. The residue was treated with iPrOH (20 mL) and stirred for 30 min at room temperature. The precipitate was filtered, washed with cold iPrOH (3×20 mL) and dried. The crude target compound was purified by HPLC Method A to provide a solid.

Yield: 31%.

¹H NMR (400 MHz, DMSO-d₆) δ 9.02 (s, 1H), 8.85 (s, 1H), 8.55 (s, 1H), 8.29 (s, 1H), 8.17 (d, J=9.1 Hz, 1H), 7.84 (d, J=5.7 Hz, 2H), 7.69 (s, 1H), 7.26 (s, 1H), 3.72 (s, 3H), 2.08 (s, 3H), 1.96 (s, 3H).

LCMS Rₜ=0.7 min using Method 1, MS ES-API calcd. for C₂₀H₂₁N₃Cl [M+H]⁺ 340.0, found 340.0.

### Example 40: Preparation of 1-(5-((7-chloroquinazolin-4-yl)amino)pyridin-2-yl)-3-methyl-1H-imidazol-3-ium iodide phosphoric acid salt (43)

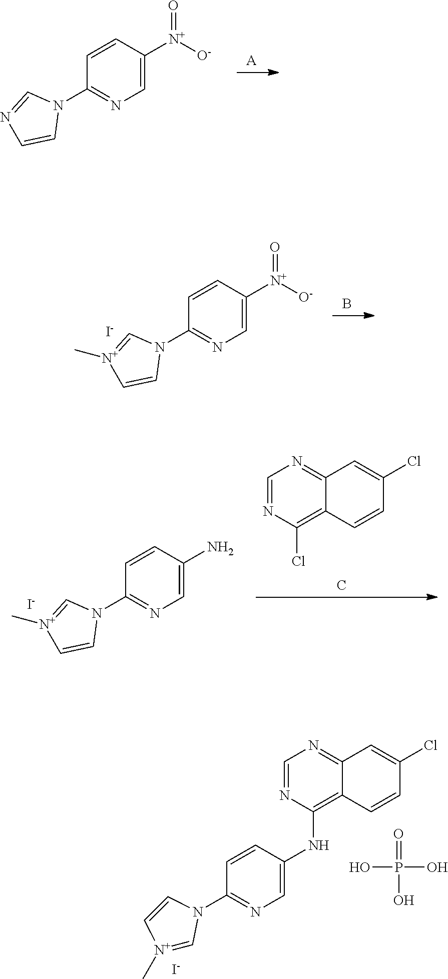

### Step A: Preparation of 3-methyl-1-(5-nitropyridin-2-yl)-1H-imidazol-3-ium iodide

2-(1H-imidazol-1-yl)-5-nitropyridine (1 mmol) and Mel (20 mmol) were dissolved in 10 mL of dry toluene and the mixture was heated with reflux condenser for 64 h. The reaction mixture was cooled, filtered and the resultant precipitate was treated with toluene (20 mL) and stirred for 2 h at room temperature. The precipitate was filtered, washed with toluene (3×20 mL) and dried. Yellow solid. Yield: 61%.

### Step B: Preparation of 1-(5-aminopyridin-2-yl)-3-methyl-1H-imidazol-3-ium iodide

To a solution of 3-methyl-1-(5-nitropyridin-2-yl)-1H-imidazol-3-ium iodide (1 mmol) in methanol (10 mL) was added 5% palladium carbon (10 mg), and the mixture was stirred under a hydrogen atmosphere at room temperature for 10 h. The reaction mixture was filtered through Celite. The filtrate was concentrated under reduced pressure and use on the next step without purification. Yield: 76%.

### Step C: Preparation of 1-(5-((7-chloroquinazolin-4-yl)amino)pyridin-2-yl)-3-methyl-1H-imidazol-3-ium iodide phosphoric acid salt

1-(5-aminopyridin-2-yl)-3-methyl-1H-imidazol-3-ium iodide (1 mmol) and 4,7-dichloroquinazoline (1 mmol) were dissolved in 10 mL of dry NMP and the mixture was heated at 120° C. for 24 h. After the completion, the reaction mixture was cooled, filtered and 20 mL of iPrOH was added. The residue was treated with iPrOH (20 mL) and H₃PO₄ (1 mmol) and stirred for 30 min at room temperature. The precipitate was filtered, washed with iPrOH (3×20 mL) and dried. The crude target compound was purified by HPLC, Method A to provide a solid. Yield: 37%.

¹H NMR (400 MHz, D₂O) δ 9.45 (s, 1H), 8.71 (d, J=2.5 Hz, 1H), 8.64 (s, 1H), 8.33 (d, J=9.0 Hz, 1H), 8.27 (dd, J=8.8, 2.5 Hz, 1H), 8.05 (d, J=2.1 Hz, 1H), 7.83 (d, J=2.0 Hz, 1H), 7.81-7.71 (m, 3H), 7.53 (s, 1H), 3.90 (s, 3H).

LCMS Rₜ=0.825 min using Method A, MS ES-API calcd. for C₁₇H₁₄N₆Cl [M+H]⁺ 338.1, found 338.0.

### Example 41: Preparation of N-(6-(1H-1,2,4-triazol-1-yl)pyridin-3-yl)-7-chloroquinolin-4-amine (44)

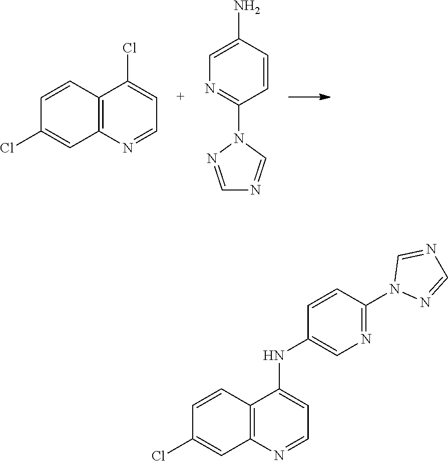 [SMILES: Clc1ccc2c(Cl)ccnc2c1 ; Nc1ccc(-n2cncn2)nc1 ; Clc1ccc2c(Nc3ccc(-n4cncn4)nc3)ccnc2c1]

4,7-dichloroquinoline (1 mmol) and 6-(1H-1,2,4-triazol-1-yl)pyridin-3-amine2 (1 mmol) were dissolved in 10 mL of dry HOAc and the mixture was heated at 90° C. for 48 h. After the completion, the reaction mixture was cooled, filtered and solvent was evaporated. The residue was treatment with iPrOH (20 mL) and stirred for 30 min at room temperature. The precipitate was filtered, washed with cold iPrOH (3×20 mL) and dried. The crude target compound was purified by HPLC Method A to provide the product as a solid. Yield: 39%.

¹H NMR (400 MHz, DMSO-d₆) δ 9.50 (s, 1H), 9.35 (s, 1H), 8.56 (dd, J=8.8, 4.1 Hz, 2H), 8.44 (d, J=9.0 Hz, 1H), 8.31 (s, 1H), 8.08 (dd, J=8.8, 2.7 Hz, 1H), 8.01-7.87 (m, 2H), 7.66 (dd, J=9.1, 2.3 Hz, 1H), 7.04 (d, J=5.5 Hz, 1H).

LCMS Rₜ=0.88 min using Method 1, MS ES-API calcd. for C₁₆H₁₁N₆Cl [M+H]⁺ 323.1, found 323.2.

### Example 42: Preparation of 7-chloro-N-(2-((dimethylamino)methyl)pyridin-4-yl)quinolin-4-amine (45)

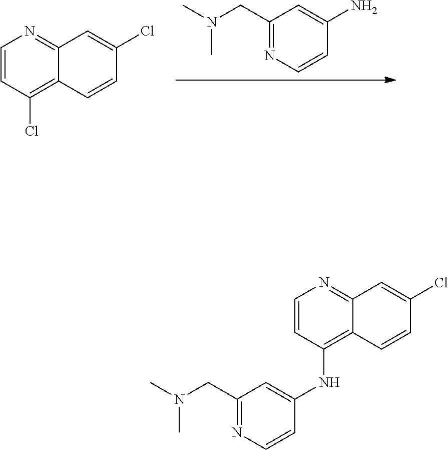

4,7-dichloroquinoline (1 mmol) and 2-((dimethylamino)methyl)pyridin-4-amine (1 mmol) were dissolved in 10 mL of dry HOAc and the mixture was heated at 90° C. for 48 h. After the completion, the reaction mixture was cooled, filtered and solvent was evaporated. The residue was treated with iPrOH (20 mL) and stirred for 30 min at room temperature. The precipitate was filtered, washed with cold iPrOH (3×20 mL) and dried. The crude target compound was purified by HPLC Method A to provide a solid. Yield: 22%.

¹H NMR (400 MHz, DMSO-d₆) δ 9.43 (s, 1H), 8.68 (d, J=5.1 Hz, 1H), 8.34 (dd, J=18.2, 7.2 Hz, 2H), 7.99 (d, J=2.3 Hz, 1H), 7.64 (dd, J=9.1, 2.3 Hz, 1H), 7.45-7.28 (m, 2H), 7.18 (d, J=5.2 Hz, 1H), 3.48 (s, 2H), 2.21 (s, 6H).

LCMS Rₜ=1.4 min using Method 1, MS ES-API calcd. for C₁₇H₁₇N4C₁ [M+H]⁺ 313.1, found 313.2.

### Biology Examples

The following examples are put forth to provide those of ordinary skill in the art with a complete disclosure and description of how to make and use the present disclosure and are not intended to limit the scope of what the inventors regard as their invention nor are they intended to represent that the experiments below are all or the only experiments performed. Efforts have been made to ensure accuracy with respect to numbers used, but some experimental errors and deviations should be accounted for. Unless indicated otherwise, parts are parts by weight, molecular weight is weight average molecular weight, temperature is in degrees Centigrade, and pressure is at or near atmospheric.

### Example 43: General Assay for Binding GBAPs to Heparin, a HS-GAG

Porcine intestinal mucosa heparin conjugated to Bovine Serum Albumin (Heparin-BSA) was prepared as described (Najjam, S. et al. 1997, Cytokine 12, 1013-1022). Heparin is a sulfated HS-GAG and is commonly used as a HS-GAG in biochemical and binding assays (Lindahl, U. and Kjellen, L. (2013) ibid). Heparin-BSA at 0.01 mg/ml in Phosphate Buffered Saline (PBS; pH 7.4) was added to a 96 well polystyrene ELISA plate (NUNC Cat. No. 449824; 100 μl per well) and incubated Over Night (ON) at 4 degrees Celsius. For negative controls, ELISA plates were coated with BSA instead of Heparin-BSA, using the same procedure. Following the incubation, the plate was washed consecutively, by immersion, with de-ionized water and PBS (pH 7.4). The ELISA plate was then blocked with nonfat milk (2%, 200 μl per well) for 2 hours at Room Temperature (RT) with gentle shaking. Following blocking, the plate was washed with de-ionized water then PBS (pH 7.4). GBAPs were dissolved and diluted in PBS (pH 6.5, supplemented with 0.1% BSA) at desired concentrations, added to the ELISA plates containing immobilized Heparin-BSA (100 μl per well) and incubated for 2 hours at RT with gentle shaking. Following incubation, the plate was washed with de-ionized water and three times with PBS (pH 6.5) plus Tween. Bound GBAP was detected by a monoclonal antibody specific for that GBAP, followed by incubation with secondary antibody conjugated to horseradish peroxidase (HRP). Antibody was diluted in PBS (pH 6.5 supplemented with 1% BSA). Following each incubation with antibody, the plate was washed with de-ionized water and three times with PBS (pH 6.5) wash buffer containing 0.1% Tween. The peroxidase substrate chromogen, TMB (Dako Cat. No. S1599) was added (100 μl per well) to the ELISA plate and incubated at room temperature. After 5 minutes ELISA Stop Solution (hydrochloric acid 1N, sulfuric acid 3N) was added (100 μl per well) to stop the peroxidase catalyzed colorimetric reaction. The Optical Density of the samples was measured at 450 nm using an ELISA plate reader (BioTek Synergy H1). Following color development, the % inhibition compared to control (BSA-coated plates) was determined. IC-50s were calculated using GraphPad Prism software.

### Example 44: Evaluation of Compounds as Inhibitors of Abeta40 and Abeta42 Binding to Heparin (HS-GAG)

The assay was performed as described in Example 43, with the following modifications. Following to coating the plates with Heparin-BSA, plates were washed as described. The compounds were dissolved in DMSO at 10 mM final concentration and further diluted prior to assay. DMSO concentration in the screening well was up to 2%. Individual compounds were co-incubated with Beta-amyloid peptides on plates containing immobilized heparin. Beta-amyloid (1-42) was purchased from rPeptide (Cat. No. A1163) and used to prepare oligomers according to Stine et al., 2003 (J Biol Chem. 278(13):11612-22) as follows. 1,1,1,3,3,3-Hexafluoro-2-propanol- (HFIP; Sigma Cat. No. 105228)-treated Beta amyloid (1-42) was dissolved in dry DMSO (Sigma Cat. No. D2650) to 5 mM concentration. Dissolved Beta amyloid (1-42) was then diluted to 100 μM in ice-cold cell culture medium (phenol red-free Ham's F-12; Caisson Labs, Cat. No. HFL05), followed immediately by vortexing for 30 seconds, and incubating at 4 degrees Celsius for 24 h. Resulting Beta amyloid (1-42) oligomers were centrifuged at 12,000 rpm for 10 min at 4 degree Celsius to remove aggregates. Oligomers were then quickly frozen and kept in aliquots at −80 degrees Celsius. Beta-amyloid (1-40) from rPeptide (Cat No. A1153) was dissolved in DMSO, quickly frozen and kept in aliquots at −80 degrees Celsius. Beta-amyloid (1-42) oligomers or Beta-amyloid (1-40) diluted in PBS (pH 6.5, supplemented with BSA, 0.1%) were added to the ELISA plate (100 μl per well) and incubated for 2 hours at RT with gentle shaking. Following the incubation, the plate was washed with de-ionized water and three times with PBS (pH 6.5) plus Tween. Bound Beta-amyloid (1-42) was detected by anti-Beta-Amyloid Monoclonal Antibody 4G8 (Biotinylated, BioLegend Cat. No. 800705), followed by Streptavidin-HRP (horseradish peroxidase, R&D System, Cat No. DY998). Bound Abeta40 was detected by anti-Beta-Amyloid Monoclonal Antibody 6E10 (Biolegend Cat. No. 803001), followed by secondary anti-IgG antibody conjugated to HRP (R&D System, Cat. No. HAF007). Following to color development with TMB, as described in Example 43, the Optical Density of the samples was measured at 450 nm using an ELISA plate reader. Following color development, the % inhibition compared to control (no compound, DMSO control) for every compound was determined.

Results: Compounds inhibited binding of Abeta (1-40) and Abeta (1-42) oligomers to heparin (HS-GAG). As described in Example 43, Heparin in the form of Heparin-BSA conjugate is used as a source of HS-GAG. The list of Inhibitor Compounds is shown in Table 2. For each compound IC-50 value in Beta-amyloid—heparin-BSA binding assay is shown in μM (micromolar). In some cases IC-50 was not measured and 00 inhibition at 30 μM compound concentration is shown instead. For simplicity, standard deviation are not shown; the Coefficient of Variation did not exceed 3000 for any IC-50 value shown. All assays were done on 96-well plates in duplicates and experiments were repeated at least twice. In the Table 2, the following abbreviations are used: A: compounds that inhibited>30% at 30 μM concentrations; B: compounds that inhibited<300% at 30 μM concentrations; NT: compounds for which inhibition curve was not obtained.

TABLE 2
Inhibition of Amyloid-beta(1-42) oligomer and
Amyloid-beta(1-40) binding to heparin (HS-GAG)
and to purified human brain membranes by Compounds.

| Com- | Abeta42/ | Abeta40/ | Abeta42/ | Abeta40/ |
|---|---|---|---|---|
| pound | Heparin | Heparin | Membrane | Membrane |
| No. | Activity | Activity | Activity | Activity |
| 1 | 2.30 | 3.67 | 2.97 | NT |
| 2 | 30.71 | 22.90 | NT | NT |
| 3 | 2.36 | 2.75 | 11.51 | NT |
| 4 | 42.68 | 51.34 | NT | NT |
| 5 | B | 3.33 | NT | NT |
| 6 | A | A | NT | NT |
| 7 | B | 25.18 | NT | NT |
| 8 | B | B | NT | NT |
| 9 | B | B | NT | NT |
| 10 | B | 35.59 | NT | NT |
| 11 | B | A | NT | NT |
| 12 | B | 17.92 | NT | NT |
| 13 | B | B | NT | NT |
| 14 | 8.14 | 7.77 | 6.03 | 18.38 |
| 15 | 18.95 | 23.01 | 7.05 | NT |
| 16 | 5.48 | 4.37 | 3.29 | 2.88 |
| 17 | 11.67 | 28.15 | NT | 27.92 |
| 18 | 13.61 | 3.31 | NT | 17.33 |
| 19 | B | 10.24 | NT | 3.09 |
| 20 | B | B | NT | NT |
| 21 | 9.50 | 7.41 | 3.30 | 5.95 |
| 22 | A | 16.31 | 3.56 | 1.78 |
| 23 | B | A | NT | NT |
| 24 | 16.88 | 6.42 | 6.67 | 24.45 |
| 25 | 9.90 | 1.70 | 7.01 | 4.68 |
| 26 | A | 10.19 | NT | 1.51 |
| 27 | A | 2.20 | 3.97 | 2.23 |
| 28 | 12.90 | 3.12 | NT | 3.71 |
| 29 | A | A | NT | NT |
| 30 | A | A | NT | NT |
| 31 | B | 23.30 | NT | NT |
| 32 | B | B | NT | NT |
| 33 | B | B | NT | NT |
| 34 | 9.93 | 34.84 | 10.43 | NT |
| 35 | 15.40 | B | 10.39 | 41.47 |
| 36 | 14.15 | 13.76 | 18.73 | NT |
| 37 | B | 45.63 | NT | NT |
| 38 | 2.74 | 1.28 | 4.62 | 0.36 |
| 39 | B | 20.69 | NT | 1.46 |
| 40 | 8.65 | B | 4.36 | NT |
| 41 | 72.45 | 32.73 | NT | 18.65 |
| 42 | B | 12.26 | NT | 6.56 |
| 43 | B | B | NT | NT |
| 44 | 35.73 | 19.26 | NT | 42.96 |
| 45 | B | B | NT | NT |

A: compounds that inhibited >30% at 30 μM concentrations.
B: compounds that inhibited <30% at 30 μM concentrations.
NT: not tested

### Example 45: Evaluation of Compounds as Inhibitors of Alpha-Synuclein Binding to Heparin (HS-GAG)

The assay was performed as described in Example 43, with the following modifications. Following to coating the plates with Heparin-BSA, plates were washed as described. The compounds were dissolved in DMSO at 10 mM final concentration and further diluted prior to assay. DMSO concentration in the screening well was up to 200. Individual compounds were co-incubated with alpha-synuclein on plates containing immobilized heparin. Alpha-synuclein purchased from rPeptide (Cat. No. S1001) was used to prepare protofibrils as described in Ihse et al., 2017. Alpha-synuclein was dissolved in PBS (pH7.4) to a concentration of 140 μM (˜2 mg/ml), and incubated for 7 days at 37° C. with rotary agitation (400 rpm). Resulting fibrils were sonicated for 10 minutes in ultrasonic water bath, quickly frozen and kept in aliquots at −80 degrees Celsius. Protofibrillar alpha-synuclein diluted in PBS (pH 6.5, supplemented with BSA, 0.1%) was added to the ELISA plate (100 μl per well) and incubated for 2 hours at RT with gentle shaking. Following the incubation, the plate was washed with de-ionized water and three times with PBS (pH 6.5) plus Tween. Bound Alpha-synuclein was detected by Anti-Alpha-synuclein Monoclonal Antibody 211 (Santa Cruz Cat. No. sc-12767), followed by secondary anti-IgG antibody conjugated to HRP (R&D System, Cat. No. HAF007). Following to color development with TMB, as described in Example 43, the Optical Density of the samples was measured at 450 nm using an ELISA plate reader. Following color development, the % inhibition compared to control (no compound, DMSO control) for every compound was determined.

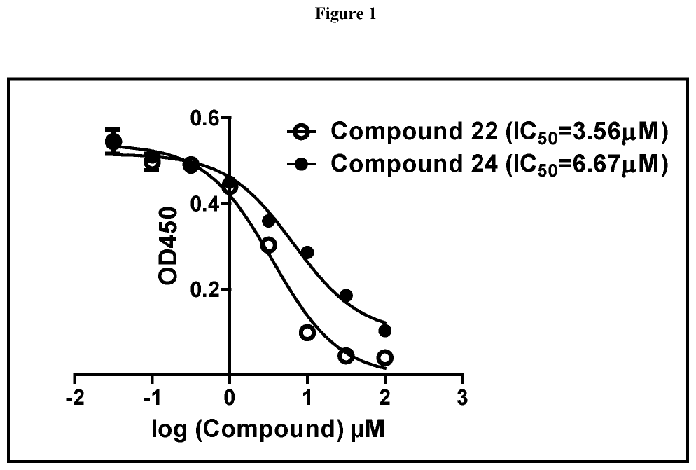

Results: Compounds inhibited binding of alpha-synuclein protofibrils to HS-GAG. An example of inhibition curve is shown in FIG. 1 for Inhibitor Compounds 22 and 24. As described in Example 43, Heparin in the form of Heparin-BSA conjugate is used as a source of HS-GAG. The list of Inhibitor Compounds is shown in Table 3. For each compound IC-50 value in alpha-synuclein protofibril—heparin-BSA binding assay is shown in μM (micromolar). In some cases IC-50 was not measured and % inhibition at 30 μM compound concentration is shown instead. For simplicity, standard deviation are not shown; the Coefficient of Variation did not exceed 30% for any IC-50 value shown. All assays were done on 96-well plates in duplicates and experiments were repeated at least twice. In the Table 3, the following abbreviations are used: A: compounds that inhibited>30% at 30 μM concentrations; B: compounds that inhibited<30% at 30 μM concentrations; NT: compounds for which inhibition curve was not obtained.

### Example 46. Evaluation of Compounds as Inhibitors of Tau Binding to Heparin (HS-GAG)

The assay was performed as described in Example 43, with the following modifications. Following to coating the plates with Heparin-BSA, plates were washed as described. The compounds were dissolved in DMSO at 10 mM final concentration and further diluted prior to assay. DMSO concentration in the screening well was up to 2%. Individual compounds were co-incubated with Tau on plates containing immobilized heparin. Tau purchased from rPeptide (Cat. No. T1001) was dissolved in DMSO (Sigma Cat. No. D2650), quickly frozen and kept in aliquots at −80 degrees Celsius. Tau dissolved in PBS (pH 6.5, supplemented with BSA, 0.1%) was added to the ELISA plate (100 μl per well) and incubated for 2 hours at RT with gentle shaking. Following the incubation, the plate was washed with de-ionized water and three times with PBS (pH 6.5) plus Tween. Bound Tau was detected by anti-Tau Monoclonal Antibody D-8 (Santa Cruz Cat. No. sc-166060), followed by secondary anti-IgG antibody conjugated to HRP (R&D System, Cat. No. HAF007). Following to color development with TMB, as described in Example 43, the Optical Density of the samples was measured at 450 nm using an ELISA plate reader. Following color development, the % inhibition compared to control (no compound, DMSO control) for every compound was determined.

TABLE 3
Inhibition of Alpha-Synuclein Protofibril binding to heparin
(HS-GAG) and to purified human brain membranes by Compounds.

| Compound | Alpha-Synuclein/ | Alpha-Synuclein/ |
|---|---|---|
| No. | Heparin Activity | Membrane Activity |
| 1 | 3.19 | 10.11 |
| 2 | B | NT |
| 3 | 1.70 | 7.34 |
| 4 | A | 10.65 |
| 5 | B | NT |
| 6 | A | 3.36 |
| 7 | 1.41 | 42.14 |
| 8 | B | NT |
| 9 | B | NT |
| 10 | B | NT |
| 11 | B | NT |
| 12 | B | NT |
| 13 | 35.23 | 4.03 |
| 14 | 2.02 | 6.51 |
| 15 | 5.22 | 3.06 |
| 16 | 3.21 | 3.25 |
| 17 | 13.52 | 13.10 |
| 18 | 1.71 | 3.73 |
| 19 | B | NT |
| 20 | B | NT |
| 21 | 2.06 | 1.80 |
| 22 | 2.87 | 10.62 |
| 23 | B | NT |
| 24 | 6.44 | 4.03 |
| 25 | 3.83 | 3.40 |
| 26 | A | NT |
| 27 | 1.95 | 7.31 |
| 28 | 3.23 | 3.17 |
| 29 | A | NT |
| 30 | A | NT |
| 31 | B | NT |
| 32 | A | NT |
| 33 | B | NT |
| 34 | 5.06 | 4.38 |
| 35 | B | NT |
| 36 | 4.17 | 9.46 |
| 37 | B | NT |
| 38 | 1.88 | 2.79 |
| 39 | 3.39 | 45.80 |
| 40 | B | 34.45 |
| 41 | 12.76 | 5.80 |
| 42 | B | NT |
| 43 | B | NT |
| 44 | 19.24 | 22.26 |
| 45 | B | NT |

A: compounds that inhibited >30% at 30 μM concentrations.
B: compounds that inhibited <30% at 30 μM concentrations.
NT: not tested

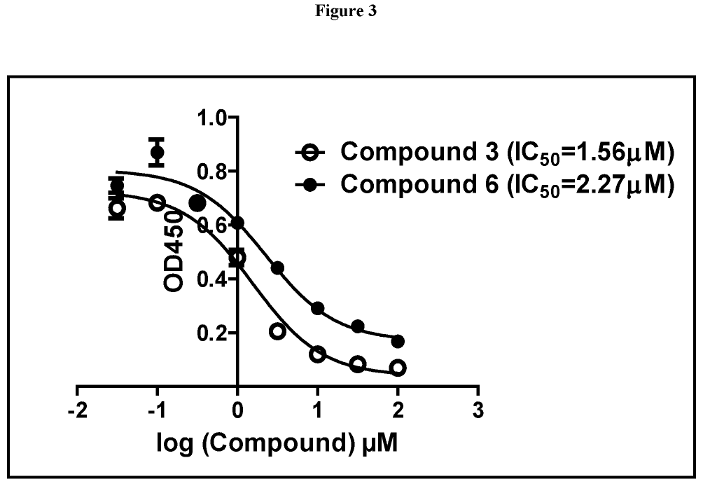

Results: Compounds inhibited binding of Tau to HS-GAG. An example of inhibition curve is shown in FIG. 3 for Inhibitor Compounds 3 and 6. As described in Example 43, Heparin in the form of Heparin-BSA conjugate is used as a source of HS-GAG. The list of Inhibitor Compounds is shown in Table 4. For each compound IC-50 value in Tau—heparin-BSA binding assay is shown in μ(micromolar). In some cases IC-50 was not measured and 00 inhibition at 30 μM compound concentration is shown instead. For simplicity, standard deviation are not shown; the Coefficient of Variation did not exceed 30% for any IC-50 value shown. All assays were done on 96-well plates in duplicates and experiments were repeated at least twice. In the Table 4, the following abbreviations are used: A: compounds that inhibited>30% at 30 μM concentrations; B: compounds that inhibited<30% at 30 μM concentrations; NT: compounds for which inhibition curve was not obtained.

TABLE 4
Inhibition of Tau binding to heparin (HS-GAG) and to
purified human brain membranes by Compounds.

| Compound | Tau/Heparin | Tau/Membrane |
|---|---|---|
| No. | Activity | Activity |
| 1 | 1.38 | 0.49 |
| 2 | 4.42 | 11.36 |
| 3 | 1.56 | 8.82 |
| 4 | 29.18 | NT |
| 5 | 31.44 | 0.84 |
| 6 | 2.27 | 0.63 |
| 7 | 15.49 | 1.22 |
| 8 | B | NT |
| 9 | B | NT |
| 10 | B | NT |
| 11 | 3.30 | 8.31 |
| 12 | 13.10 | 29.87 |
| 13 | B | NT |
| 14 | 20.30 | 1.86 |
| 15 | 67.90 | NT |
| 16 | 3.92 | 9.61 |
| 17 | 12.22 | NT |
| 18 | 13.64 | NT |
| 19 | 46.65 | NT |
| 20 | B | NT |
| 21 | 80.88 | NT |
| 22 | 28.24 | NT |
| 23 | 25.73 | NT |
| 24 | 37.95 | 2.56 |
| 25 | B | NT |
| 26 | 17.73 | NT |
| 27 | 2.86 | NT |
| 28 | 61.79 | NT |
| 29 | 32.40 | NT |
| 30 | 32.16 | NT |
| 31 | 33.71 | NT |
| 32 | B | NT |
| 33 | 24.71 | NT |
| 34 | B | NT |
| 35 | 41.31 | NT |
| 36 | 28.51 | NT |
| 37 | B | NT |
| 38 | 2.94 | 4.86 |
| 39 | 13.24 | 54.10 |
| 40 | A | 65.14 |
| 41 | 14.73 | 5.58 |
| 42 | B | NT |
| 43 | B | NT |
| 44 | A | NT |
| 45 | B | NT |

A: compounds that inhibited >30% at 30 μM concentrations.
B: compounds that inhibited <30% at 30 μM concentrations.
NT: not tested

### Example 47. Evaluation of Compounds as Inhibitors of TDP-43 Binding to Heparin (HS-GAG)

The assay was performed as described in Example 43, with the following modifications.

Following to coating the plates with Heparin-BSA, plates were washed as described. The compounds were dissolved in DMSO at 10 mM final concentration and further diluted prior to assay. DMSO concentration in the screening well was up to 2%. Individual compounds were co-incubated with TDP-43 on plates containing immobilized heparin. TDP43 from R&D System (Cat. No. AP-190) was quickly frozen and kept in aliquots at −80 degrees Celsius. TDP-43 dissolved in PBS (pH 6.5, supplemented with BSA, 0.1%) was added to the ELISA plate (100 μl per well) and incubated for 2 hours at RT with gentle shaking. Following the incubation, the plate was washed with de-ionized water and three times with PBS (pH 6.5) plus Tween. Bound TDP-43 was detected by Anti-TDP43 Monoclonal Antibody (R&D System Cat. No. MAB7778), followed by secondary anti-IgG antibody conjugated to HRP (R&D System, Cat. No. HAF007). Following to color development with TMB, as described in Example 43, the Optical Density of the samples was measured at 450 nm using an ELISA plate reader. Following color development, the % inhibition compared to control (no compound, DMSO control) for every compound was determined.

Results: Compounds inhibited binding of TDP-43 to HS-GAG. As described in Example 43, Heparin in the form of Heparin-BSA conjugate is used as a source of HS-GAG. The list of Inhibitor Compounds is shown in Table 5 above. For each compound IC-50 value in TDP-43—heparin-BSA binding assay is shown in μM (micromolar). In some cases IC-50 was not measured and % inhibition at 30 μM compound concentration is shown instead. For simplicity, standard deviation are not shown; the Coefficient of Variation did not exceed 30% for any IC-50 value shown. All assays were done on 96-well plates in duplicates and experiments were repeated at least twice. In the Table, the following abbreviations are used: A: compounds that inhibited>30% at 30 μM concentrations; B: compounds that inhibited<30% at 30 μM concentrations; NT: compounds for which inhibition curve was not obtained.

TABLE 5
Inhibition of TDP-43 binding to heparin (HS-GAG) and
to purified human brain membranes by Compounds.

| Compound | TDP43/Heparin | TDP43/Membrane |
|---|---|---|
| No. | Activity | Activity |
| 1 | 2.92 | 3.42 |
| 2 | 61.65 | NT |
| 3 | 8.10 | 6.57 |
| 4 | B | NT |
| 5 | 71.41 | 14.07 |
| 6 | B | NT |
| 7 | B | NT |
| 8 | 10.78 | 25.16 |
| 9 | B | NT |
| 10 | A | NT |
| 11 | B | NT |
| 12 | B | NT |
| 13 | B | NT |
| 14 | 4.32 | 4.01 |
| 15 | 3.06 | 3.01 |
| 16 | 2.16 | 2.11 |
| 17 | 33.57 | 22.47 |
| 18 | 4.77 | 9.28 |
| 19 | 36.83 | NT |
| 20 | B | NT |
| 21 | 4.60 | 7.12 |
| 22 | 9.02 | 12.23 |
| 23 | B | NT |
| 24 | 0.89 | 4.00 |
| 25 | 2.45 | 11.49 |
| 26 | 32.83 | 27.23 |
| 27 | 2.18 | 2.98 |
| 28 | 5.73 | 8.93 |
| 29 | B | NT |
| 30 | 12.19 | NT |
| 31 | B | NT |
| 32 | B | NT |
| 33 | B | NT |
| 34 | 5.23 | 3.67 |
| 35 | 15.63 | 13.08 |
| 36 | 47.91 | 48.62 |
| 37 | B | NT |
| 38 | 5.73 | 6.17 |
| 39 | A | NT |
| 40 | 7.42 | 5.28 |
| 41 | 4.25 | 8.32 |
| 42 | B | NT |
| 43 | B | NT |
| 44 | 6.54 | 26.18 |
| 45 | B | NT |

A: compounds that inhibited >30% at 30 μM concentrations.
B: compounds that inhibited <30% at 30 μM concentrations.
NT: not tested

### Example 48: Evaluation of Compounds as Inhibitors of SAA Binding to Heparin (HS-GAG)

The assay was performed as described in Example 43, with the following modifications. Following to coating the plates with Heparin-BSA, plates were washed as described. The compounds were dissolved in DMSO at 10 mM final concentration and further diluted prior to assay. DMSO concentration in the screening well was up to 2%. Individual compounds were co-incubated with SAA on plates containing immobilized heparin. Serum amyloid A (SAA) from PeProtech (NJ, USA, Cat. No. 300-53) was dissolved in DMSO, quickly frozen and kept in aliquots at −80 degrees Celsius. SAA dissolved in Tris buffer (pH 6.5, supplemented with BSA, 0.1%) was added to the ELISA plate (100 μl per well) and incubated for 2 hours at RT with gentle shaking. Following the incubation, the plate was washed with de-ionized water and three times with Tris Buffered Saline (TBS, pH 6.5) plus Tween. Bound SAA was detected by Anti-SAA Monoclonal Antibody (R&D System Cat. No. MAB30192), followed by secondary anti-IgG antibody conjugated to HRP (R&D System, Cat. No. HAF007). Antibody was diluted in TBS antibody buffer (pH6.5 supplemented with 1% BSA). Following each incubation with antibody, the plate was washed with de-ionized water and three times with TBS (pH6.5) wash buffer containing 0.1% Tween. Following to color development with TMB, as described in Example 43, the Optical Density of the samples was measured at 450 nm using an ELISA plate reader. Following color development, the % inhibition compared to control (no compound, DMSO control) for every compound was determined.

Results: Compounds inhibited binding of SAA to HS-GAG. As described in Example 43, Heparin in the form of Heparin-BSA conjugate is used as a source of HS-GAG. The list of Inhibitor Compounds is shown in Table 6. For each compound IC-50 value in SAA—heparin-BSA binding assay is shown in μM (micromolar). In some cases IC-50 was not measured and % inhibition at 3 μM compound concentration is shown instead. For simplicity, standard deviation are not shown; the Coefficient of Variation did not exceed 30% for any IC-50 value shown. All assays were done on 96-well plates in triplicates and experiments were repeated at least twice. In the Table, the following abbreviations are used: A: compounds that inhibited>30% at 30 μM concentrations; B: compounds that inhibited<30% at 30 μM concentrations; NT: compounds for which inhibition curve was not obtained.

TABLE 6
Inhibition of SAA binding to heparin (HS-GAG) and to
purified human brain membranes by Compounds.

| Compound | Serum Amyloid A/ | Serum Amyloid A/ |
|---|---|---|
| No. | Heparin Activity | Membrane Activity |
| 1 | 8.16 | 12.32 |
| 2 | 16.04 | NT |
| 3 | A | NT |
| 4 | B | NT |
| 5 | 24.04 | NT |
| 6 | A | NT |
| 7 | A | NT |
| 8 | B | NT |
| 9 | B | NT |
| 10 | B | NT |
| 11 | B | NT |
| 12 | 31.73 | NT |
| 13 | B | 44.52 |
| 14 | 7.67 | 27.53 |
| 15 | 12.32 | 32.52 |
| 16 | 13.94 | 9.93 |
| 17 | 34.87 | NT |
| 18 | 22.17 | 24.01 |
| 19 | 8.59 | NT |
| 20 | A | NT |
| 21 | 85.24 | 47.84 |
| 22 | 83.24 | NT |
| 23 | B | NT |
| 24 | 7.89 | 8.22 |
| 25 | 40.92 | 22.04 |
| 26 | B | NT |
| 27 | 24.66 | 14.00 |
| 28 | 8.78 | 22.86 |
| 29 | B | NT |
| 30 | B | NT |
| 31 | A | 70.79 |
| 32 | A | NT |
| 33 | B | NT |
| 34 | 12.42 | 8.66 |
| 35 | 31.80 | NT |
| 36 | 22.01 | 15.27 |
| 37 | B | NT |
| 38 | 14.92 | 23.60 |
| 39 | A | NT |
| 40 | A | NT |
| 41 | 12.16 | 13.21 |
| 42 | B | NT |
| 43 | 31.46 | NT |
| 44 | 17.39 | 11.43 |
| 45 | 63.52 | NT |

A: compounds that inhibited >30% at 30 μM concentrations.
B: compounds that inhibited <30% at 30 μM concentrations.
NT: not tested

### Example 49. General Assay for Binding GBAPs to Human Brain Cell Membranes

Purified human brain cell membranes were prepared by differential centrifugation as follows. Five grams of frozen human brain postmortem tissue (obtained from University of Kentucky Alzheimer's Disease Center Tissue Bank) was homogenized in 50 ml HEPES-Buffered sucrose (0.32M sucrose, 4 mM HEPES pH7.4, protease inhibitors) with a motor driven glass-teflon homogenizer. After removing nuclear and cell debris by centrifugation of homogenates at 1,000×g for 10 min at 4 degree Celsius, post-nuclear supernatant was centrifuged at 100,000×g for 30 minutes to yield the crude membrane pellet. Crude membrane pellet was re-suspended in 0.32M sucrose, then centrifuged again at 100,000×g for 30 minutes to yield washed membrane pellet. Membrane pellet was resuspended in sterile PBS (pH7.4), quickly frozen, and kept in aliquots at −80 degrees Celsius. This preparation contains human brain cell membranes. Protein concentration was measured with BCA protein assay kit (Pierce Cat. No. 23227). Purified brain membranes were diluted to 0.01 mg/ml in PBS (pH 7.4) and then added to a 96 well polystyrene ELISA plate (NUNC Cat. No. 449824; 100 μl per well). Plates were incubated Over Night (ON) at 4 degrees Celsius. Following the incubation, the plate was washed consecutively, by immersion, with de-ionized water and PBS (pH 7.4). The ELISA plate was then blocked with nonfat milk (2%, 200 μl per well) for 2 hours at Room Temperature (RT). Following blocking, the plate was washed with de-ionized water then PBS (pH 7.4). GBAPs were dissolved and diluted in PBS (pH 6.5, supplemented with 0.1% BSA) at desired concentrations, added to the ELISA plates coated with brain membranes (100 μl per well) and incubated for 2 hours at RT with gentle shaking. Following incubation, the plate was washed with de-ionized water and three times with PBS (pH 6.5) plus Tween. Bound GBAP was detected by a monoclonal antibody specific for that GBAP, followed by incubation with secondary antibody conjugated to horseradish peroxidase (HRP). Antibody was diluted in PBS (pH6.5 supplemented with 1% BSA). Following each incubation with antibody, the plate was washed with de-ionized water and three times with PBS (pH6.5) wash buffer containing 0.1% Tween. The peroxidase substrate chromogen, TMB (Dako Cat. No. S1599) was added (100 μl per well) to the ELISA plate and incubated at room temperature. After 5 minutes ELISA Stop Solution (hydrochloric acid 1N, sulfuric acid 3N) was added (100 μl per well) to stop the peroxidase catalyzed colorimetric reaction. The Optical Density of the samples was measured at 450 nm using an ELISA plate reader (BioTek Synergy H₁). Following color development, the % inhibition compared to control was determined. IC-50s were calculated using GraphPad Prism software.

### Example 50: Evaluation of Compounds as Inhibitors of Abeta40 and Abeta42 Binding to Human Brain Cell Membranes

The assay was performed as described in Example 49, with the following modifications. Following to coating the plates with purified human brain cell membranes, plates were washed as described. The compounds were dissolved in DMSO at 10 mM final concentration and further diluted prior to assay. DMSO concentration in the screening well was up to 2%. Individual compounds were co-incubated with Beta-amyloid peptides on plates coated with purified human brain membranes. Beta-amyloid (1-42) oligomers were prepared as described in Example 2. Beta-amyloid (1-40) from rPeptide (Cat No. A1153) was dissolved in DMSO, quickly frozen and kept in aliquots at −80 degrees Celsius. Beta-amyloid (1-42) oligomers or Beta-amyloid (1-40) dissolved in PBS (pH 6.5, supplemented with BSA, 0.1%) were added to the ELISA plate (100 μl per well) and incubated for 2 hours at RT with gentle shaking. Following the incubation, the plate was washed with de-ionized water and three times with PBS (pH 6.5) plus Tween. Bound Beta-amyloid was detected by an anti-Beta-Amyloid Monoclonal Antibody 4G8 (Biotinylated, BioLegend Cat. No. 800705) for Beta-amyloid (1-42), an anti-Beta-Amyloid Monoclonal Antibody 6E10 (Biolegend Cat. No. 803001) for Beta-amyloid (1-40), followed by Streptavidin-HRP (horseradish peroxidase, R&D System, Cat No. DY998) for biotinylated 4G8 antibody and secondary anti-IgG antibody conjugated to HRP (R&D System, Cat. No. HAF007) for 6E10 antibody. Following to color development with TMB, as described in Example 49, the Optical Density of the samples was measured at 450 nm using an ELISA plate reader. Following color development, the % inhibition compared to control (no compound, DMSO control) for every compound was determined.

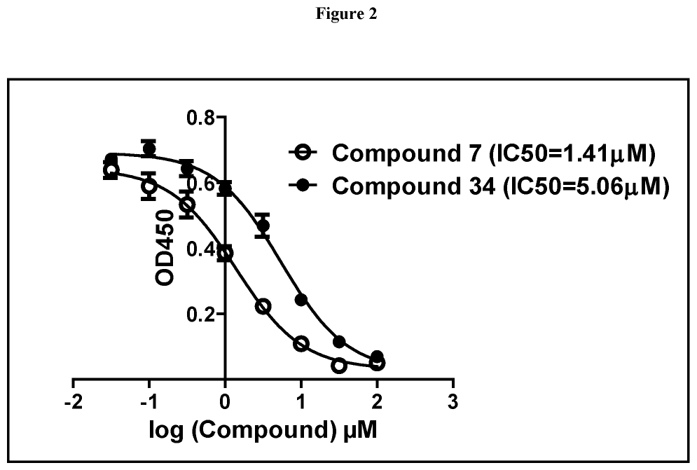

Results: Compounds inhibited binding of Abeta (1-40) and Abeta (1-42) oligomers to purified human brain cell membranes. An example of inhibition curve is shown in FIG. 2 for Inhibitor Compounds 7 and 34. The list of Inhibitor Compounds is shown in Table 2. For each compound IC-50 value is shown in μM (micromolar). In some cases IC-50 was not measured and % inhibition at 3 μM compound concentration is shown instead. For simplicity, standard deviation are not shown; the Coefficient of Variation did not exceed 30% for any IC-50 value shown. All assays were done on 96-well plates in duplicates and experiments were repeated at least twice. In the Table, the following abbreviations are used: A: compounds that inhibited>30% at 30 μM concentrations; B: compounds that inhibited<30% at 30 μM concentrations; NT: compounds for which inhibition curve was not obtained.

### Example 51: Evaluation of Compounds as Inhibitors of Protofibrillar Alpha-Synuclein Binding to Purified Human Brain Cell Membranes

The assay was performed as described in Example 49, with the following modifications. Following to coating the plates with human brain membranes, plates were washed as described. The compounds were dissolved in DMSO at 10 mM final concentration and further diluted prior to assay. DMSO concentration in the screening well was up to 2%. Individual compounds were co-incubated with alpha-synuclein on plates coated with brain membranes. Protofibrillar Alpha-synuclein were prepared as described in Example 45. Protofibrillar Alpha-synuclein diluted in PBS (pH 6.5, supplemented with BSA, 0.1%) was added to the ELISA plate (100 μl per well) and incubated for 2 hours at RT with gentle shaking. Following the incubation, the plate was washed with de-ionized water and three times with PBS (pH 6.5) plus Tween. Bound alpha-synuclein was detected by Anti-Alpha-synuclein Monoclonal Antibody 211 (Santa Cruz Cat. No. sc-12767), followed by secondary anti-IgG antibody conjugated to HRP (R&D System, Cat. No. HAF007). Following to color development with TMB, as described in Example 49, the Optical Density of the samples was measured at 450 nm using an ELISA plate reader. Following color development, the % inhibition compared to control (no compound, DMSO control) for every compound was determined.

Results: Compounds inhibited binding of Protofibrillar Alpha-synuclein to purified human brain cell membranes. The list of Inhibitor Compounds is shown in Table 3. For each compound IC-50 value is shown in μM (micromolar). In some cases IC-50 was not measured and % inhibition at 30 μM compound concentration is shown instead. For simplicity, standard deviation are not shown; the Coefficient of Variation did not exceed 30% for any IC-50 value shown. All assays were done on 96-well plates in duplicates and experiments were repeated at least twice. In the Table, the following abbreviations are used: A: compounds that inhibited>30% at 30 μM concentrations; B: compounds that inhibited<30% at 30 μM concentrations; NT: compounds for which inhibition curve was not obtained.

### Example 52: Evaluation of Compounds as Inhibitors of Tau Binding to Human Brain Cell Membranes

The assay was performed as described in Example 49, with the following modifications.

Following to coating the plates with purified human brain cell membranes, plates were washed as described. The compounds were dissolved in DMSO at 10 mM final concentration and further diluted prior to assay. DMSO concentration in the screening well was up to 2%. Individual compounds were co-incubated with Tau on plates coated with purified human brain membranes. Tau purchased from rPeptide (Cat. No. T1001) was dissolved in DMSO (Sigma Cat. No. D2650), quickly frozen and kept in aliquots at −80 degrees Celsius. Tau dissolved in PBS (pH 6.5, supplemented with BSA, 0.1%) was added to the ELISA plate (100 μl per well) and incubated for 2 hours at RT with gentle shaking. Following the incubation, the plate was washed with de-ionized water and three times with PBS (pH 6.5) plus Tween. Bound Tau was detected by anti-Tau Monoclonal Antibody D-8 (Santa Cruz Cat. No. sc-166060), followed by secondary anti-IgG antibody conjugated to HRP (R&D System, Cat. No. HAF007). Following to color development with TMB, as described in Example 49, the Optical Density of the samples was measured at 450 nm using an ELISA plate reader. Following color development, the % inhibition compared to control (no compound, DMSO control) for every compound was determined.

Results: Compounds inhibited binding of Tau to purified human brain cell membranes. The list of Inhibitor Compounds is shown in Table 4. For each compound IC-50 value is shown in μM (micromolar). In some cases IC-50 was not measured and % inhibition at 30 μM compound concentration is shown instead. For simplicity, standard deviation are not shown; the Coefficient of Variation did not exceed 30% for any IC-50 value shown. All assays were done on 96-well plates in duplicates and experiments were repeated at least twice. In the Table, the following abbreviations are used: A: compounds that inhibited>30% at 30 μM concentrations; B: compounds that inhibited<30% at 30 μM concentrations; NT: compounds for which inhibition curve was not obtained.

### Example 53: Evaluation of Compounds as Inhibitors of TDP-43 Binding to Purified Human Brain Cell Membranes

The assay was performed as described in Example 49, with the following modifications. Following to coating the plates with purified human brain cell membranes, plates were washed as described. The compounds were dissolved in DMSO at 10 mM final concentration and further diluted prior to assay. DMSO concentration in the screening well was up to 2%. Individual compounds were co-incubated with TDP-43 on plates coated with brain membranes. TDP43 from R&D System (Cat. No. AP-190) was quickly frozen and kept in aliquots at −80 degrees Celsius. TDP-43 dissolved in PBS (pH 6.5, supplemented with BSA, 0.1%) was added to the ELISA plate (100 μl per well) and incubated for 2 hours at RT with gentle shaking. Following the incubation, the plate was washed with de-ionized water and three times with PBS (pH 6.5) plus Tween. Bound TDP-43 was detected by Anti-TDP43 Monoclonal Antibody (R&D System Cat. No. MAB7778), followed by secondary anti-IgG antibody conjugated to HRP (R&D System, Cat. No. HAF007). Following to color development with TMB, as described in Example 49, the Optical Density of the samples was measured at 450 nm using an ELISA plate reader. Following color development, the % inhibition compared to control (no compound, DMSO control) for every compound was determined.

Results: Compounds inhibited binding of TDP-43 to purified human brain cell membranes. The list of Inhibitor Compounds is shown in Table 5. For each compound IC-50 value is shown in μM (micromolar). In some cases IC-50 was not measured and % inhibition at 30 μM compound concentration is shown instead. For simplicity, standard deviation are not shown; the Coefficient of Variation did not exceed 25% for any IC-50 value shown. All assays were done on 96-well plates in triplicates and experiments were repeated at least twice. In the Table, the following abbreviations are used: A: compounds that inhibited>30% at 30 μM concentrations; B: compounds that inhibited<30% at 30 μM concentrations; NT: compounds for which inhibition curve was not obtained.

### Example 54: Evaluation of Compounds as Inhibitors of SAA Binding to Purified THP-1 Cell Membranes

THP-1 (human acute monocyte leukemia cell line; ATCC cat. no. TIB-202) cell membranes were prepared by differential centrifugation as follows. THP-1 cell was grown in RPMI 1640 media (GIBCO Cat. No. 21875034, supplemented with 10% FBS) at 37 degree Celsius 5% CO₂ atmosphere, harvested, and stored at −80 degree Celsius. Frozen THP-1 cells were homogenized in HEPES-Buffered sucrose (0.32M sucrose, 4 mM HEPES pH7.4, protease inhibitors) with a motor driven glass-teflon homogenizer. After removing nuclear and cell debris by centrifugation of homogenates at 1,000×g for 10 min at 4 degree Celsius, post-nuclear supernatant was centrifuged at 10,000×g for 15 minutes to remove mitochondrial fraction. Resulting supernatant was centrifuged at 100,000×g for 30 minutes to yield the crude membrane pellet. Crude membrane pellet was re-suspended in 0.32M sucrose, then centrifuged again at 100,000 xg for 30 minutes to yield washed membrane pellet. Membrane pellet was resuspended in sterile PBS (pH7.4), quickly frozen and kept in aliquots at −80 degrees Celsius. This preparation contains mostly cell membranes. Protein concentration was measured with BCA protein assay kit (Pierce Cat. No. 23227). The binding assay was performed as described in Example 48, with the following modifications. Following to coating the plates with purified human THP-1 cell membranes at 0.01 mg/ml concentration, plates were washed and blocked with 2% milk as described. The compounds were dissolved in DMSO at 10 mM final concentration and further diluted prior to assay. DMSO concentration in the screening well was up to 2%. Individual compounds were co-incubated with SAA on plates coated with cell membranes. SAA from PreProtech (NJ, USA, Cat. No. 300-53) was dissolved in DMSO, quickly frozen and kept in aliquots at −80 degrees Celsius. SAA dissolved in Tris Buffer (pH 6.5, supplemented with BSA, 0.1%) was added to the ELISA plate (100 μl per well) and incubated for 2 hours at RT with gentle shaking. Following the incubation, the plate was washed with de-ionized water and three times with TBS (pH 6.5) plus Tween. Bound SAA was detected by Anti-SAA Monoclonal Antibody (R&D System Cat. No. MAB30192, followed by secondary anti-IgG antibody conjugated to HRP (R&D System, Cat. No. HAF007). Antibody was diluted in TBS antibody buffer (pH6.5 supplemented with 1% BSA). Following each incubation with antibody, the plate was washed with de-ionized water and three times with TBS (pH6.5) wash buffer containing 0.1% Tween. Following to color development with TMB, as described in Example 49, the Optical Density of the samples was measured at 450 nm using an ELISA plate reader. Following color development, the % inhibition compared to control (no compound, DMSO control) for every compound was determined.

Results: Compounds inhibited binding of SAA to purified THP-1 cell membranes. The list of Inhibitor Compounds is shown in Table 6. For each compound IC-50 value is shown in μM (micromolar). In some cases IC-50 was not measured and % inhibition at 30 μM compound concentration is shown instead. For simplicity, standard deviation are not shown; the Coefficient of Variation did not exceed 30% for any IC-50 value shown. All assays were done on 96-well plates in duplicates and experiments were repeated at least twice. In the Table, the following abbreviations are used: A: compounds that inhibited>30% at 30 μM concentrations; B: compounds that inhibited<30% at 30 μM concentrations; NT: compounds for which inhibition curve was not obtained.

### Example 55: An Assay to Demonstrate Direct Interaction of Inhibitor Compounds with Heparin and Other GAGs

In order to demonstrate that the Inhibitor Compounds bind directly to heparin and other GAGs, individual compounds were incubated with immobilized heparin in the absence of Beta-Amyloid (1-42). 96 well ELISA plates were coated with Heparin-BSA, then blocked with BSA as described in Example 43 and 44. If other GAGs are to be tested, they can be directly or indirectly (e.g., via GAG-BSA conjugate) immobilized to a plate, as described in Example 43. Beta-amyloid inhibitor compounds, at final concentration 0.1-200 μM, were incubated in the ELISA plate for 90 min, and then washed with incubation buffer (pre-incubation). After washing, Beta-amyloid (1-42) was added to the wells pre-incubated with compounds. At the same time, in separate control wells, Beta-amyloid (1-42) was co-incubated with Beta-amyloid (1-42) inhibitor compounds for 90 min. (co-incubation). Following the incubation, Beta-amyloid (1-42) bound to the plate was quantified by anti-Beta-amyloid antibody followed by antibody conjugated to Horse Radish Peroxidase, and OD measurement as described in Example 44.

Results: Several beta-amyloid-heparin inhibitor compounds inhibited Beta-amyloid (1-42) binding to similar extent in pre-incubation vs. co-incubation experiments. Without being bound by theory, these compounds may interact with GAGs other than heparin, based on the structural similarity of heparin to other GAGs, especially HS-GAGs.

### Example 56: Assay for Pro-IAPP Binding to Immobilized Heparin that is Suitable for the Screening of Compound Collections

A 96-well plate assay for pro-IAPP interaction with heparin, that is suitable for drug screening, was developed. We used pro-IAPP (1-48), because it was reported to have a heparin binding domain whereas mature IAPP may not have one (Park K, Verchere CB. J Biol Chem. 2001, 276(20):16611-6). The assay measures binding of pro-IAPP (1-48) (custom-synthesized) to immobilized heparin-BSA). The amount of bound pro-IAPP was determined by an ELISA assay using a polyclonal anti-IAPP antibody, followed by quantitative color development of second antibody-conjugated horseradish peroxidase, in an assay similar to the one described in Example 43. The pro-IAPP-heparin assay was then used to screen a collection of about 2,500 compounds on 96-well plates. For this purpose, the compounds at a final concentration of 30 microM, were co-incubated with pro-IAPP on plates containing immobilized heparin. Following color development, the % inhibition for every compound was determined. Positive and negative controls were included on every plate. Compounds which inhibited at least 30% of the signal were scored as hits.

Results: Screening identified about 12 hits that inhibited at least 30% of signal and could be validated in repeat assays. These compounds had diverse chemical structures, and were deemed suitable for lead optimization.

It may be found upon examination that additional species and genera not presently excluded from the claims to pharmaceutical compositions and chemical compounds are not patentable to the inventors in this application. In that case, the subsequent exclusion of species and genera in applicants' claims are to be considered artifacts of patent prosecution and not reflective of the inventors' concept or description of their invention. The invention, in a composition aspect, is all compounds of formula I except those that are in the public's possession.

It is to be understood that the invention is not limited to the particular embodiments of the invention described above, as variations of the particular embodiments may be made and still fall within the scope of the appended claims.

What is claimed is:

1. A compound of the general formula I: 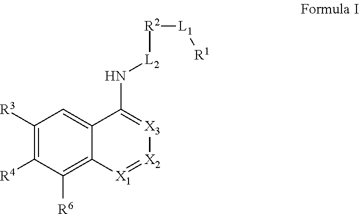
and a pharmaceutically acceptable diluent or carrier,
wherein
X₁, X₂, and X₃ are independent and optionally —CR⁹— or N; R⁹ is alkyl, halogen, —O-alkyl. If X₁ is CR⁹ then R⁹ and R⁶ can be joined to form a 5-7 member carbocyclic, heteroaromatic or heterocyclic ring;
R³ and R⁴ are independent and optionally H, halogen, —O-alkyl. R3 and R4 can be joined to form a fused phenyl;
R⁵ is H, C1-6 alkyl;
R² is phenyl optionally substituted with C1-6 alkyl, —OH, —O-alkyl, halogen, heterocycle, heteroaromatic; 5 or 6 member heterocycle or heteroaromatic optionally substituted with C1-6 alkyl, —OH, —O-alkyl, halogen; a 5 or 6-member heterocycle optionally substituted with alkyl, —O-alkyl, halogen, heterocycle, heteroaromatic; —N+(CH₃)3, optionally substituted 4, 5 and 6-member heterocycle including heterocycles and heteroaromatics that may contain a quaternized nitrogen to form a charged species:
L₁ is a bond, —CH₃, —CH₂—, —[CR7R8]n- where R7 and R8 are independently: F, C1-6 alkyl, joined to form a C3-6 carbocycle or heterocycle, or heteroaromatic
n=0, 1, 2, 3;
L₂ can be a bond or —N═C(R5)-;
R₁ is null, a 5 or 6-member heterocycle optionally substituted with alkyl, —O-alkyl, halogen; —N+(CH₃)3, optionally substituted 4, 5 and 6-member heterocycle including heterocycles and heteroaromatics that may contain a quaternized nitrogen to form a charged species;
R₆ is alkyl;
and pharmaceutically acceptable salts thereof. Also included are all the geometrical isomers about the carbon-nitrogen double bonds and possible tautomers.

2. The compound of claim 1, selected from the group consisting of:
(E)-4-((E)-(4-(1H-imidazol-1-yl)-2-methylbenzylidene)hydrazono)-7-chloro-1,4-dihydroquinoline (Compound 1)
(E)-7-chloro-4-((E)-(4-(4,5-dimethyl-1H-imidazol-1-yl)benzylidene)hydrazono)-1,4-dihydroquinoline (Compound 2)
(E)-4-((E)-(4-(1H-pyrazol-1-yl)benzylidene)hydrazono)-7-chloro-1,4-dihydroquinoline (Compound 3)
(Z)-1-(4-((2-(6-chloroquinolin-4-yl)hydrazineylidene)methyl)phenyl)—N,N,N-trimethylmethanaminium iodide (Compound 4)
(E)-4-(2-(4-(1H-imidazol-1-yl)benzylidene)hydrazinyl)-7-fluoroquinoline (Compound 5)
(E)-4-(4-((2-(7-fluoroquinolin-4-yl)hydrazono)methyl)benzyl)morpholine (Compound 6)
(E)-7-chloro-4-(2-(4-((4-methylpiperazin-1-yl)methyl)benzylidene)hydrazinyl)quinolone (Compound 7)
(E)-1-(4-((2-(1,2-dihydroacenaphthylen-5-yl)hydrazineylidene)methyl)phenyl)-1H-imidazole (Compound 8) (Z)-1-methyl-4-((2-(quinolin-4-yl)hydrazono)methyl)pyridin-1-ium iodide (Compound 9)
(E)-4-(4-((2-(7-fluoroquinolin-4-yl)hydrazono)methyl)benzyl)thiomorpholine 1,1-dioxide (Compound 10)
4-(2-(4-(1H-imidazol-1-yl)-3-methylbenzylidene)hydrazineyl)-6,7-dimethoxyquinoline hydrochloride (Compound 11)
4-(2-(4-(1H-imidazol-1-yl)-3-methylbenzylidene)hydrazineyl)-7-methoxyquinoline (Compound 12)
(E)-4-(2-(4-(1H-imidazol-1-yl)-3-methoxybenzylidene)hydrazineyl)-6,7-dimethoxyquinoline phosphate (Compound 13)
(E)-4-(2-((6-(1H-imidazol-1-yl)pyridin-3-yl)methylene)hydrazineyl)-7-chloroquinoline hydrochloride (Compound 14)
7-chloro-4-(2-((6-(4,5-dimethyl-1H-imidazol-1-yl)pyridin-3-yl)methylene)hydrazineyl)quinazoline hydrochloride (Compound 15)
7-chloro-4-(2-(1-(6-(4,5-dimethyl-1H-imidazol-1-yl)pyridin-3-yl)ethylidene)hydrazineyl)quinazoline phosphate (Compound 16)
(E)-1-(5-(1-(2-(7-chloroquinazolin-4-yl)hydrazineylidene)ethyl)pyridin-2-yl)-3-methyl-1H-imidazol-3-ium iodide phosphoric acid salt (Compound 17)
(E)-7-chloro-4-(2-(1-(6-(2,4-dimethyl-1H-imidazol-1-yl)pyridin-3-yl)ethylidene)hydrazineyl)quinazoline phosphoric acid salt (Compound 18)
(E)-4-(2-(1-(6-(2,4-dimethyl-1H-imidazol-1-yl)pyridin-3-yl)ethylidene)hydrazineyl)quinazoline (Compound 19)
7-chloro-N-(2-(4-methylpiperazin-1-yl)pyridin-4-yl)quinolin-4-amine (Compound 20)
(E)-4-(2-(1-(6-(4,5-dimethyl-1H-imidazol-1-yl)pyridin-3-yl)ethylidene)hydrazineyl)-7-fluoroquinazoline (Compound 21)
7-chloro-4-(2-(1-(6-(4,5-dimethyl-1H-imidazol-1-yl)pyridin-3-yl)ethylidene)hydrazineyl)quinoline (Compound 22)
1-(5-(1-(2-(7-chloroquinolin-4-yl)hydrazineylidene)ethyl)pyridin-2-yl)-3-methyl-1H-imidazol-3-ium iodide (Compound 23)
(E)-6-chloro-1-(2-(1-(6-(2,4-dimethyl-1H-imidazol-1-yl)pyridin-3-yl)ethylidene)hydrazineyl)phthalazine (Compound 24)
7-chloro-N-(6-(4,5-dimethyl-1H-imidazol-1-yl)pyridin-3-yl)quinazolin-4-amine (Compound 25)
(E)-1-(5-(1-(2-(7-fluoroquinazolin-4-yl)hydrazineylidene)ethyl)pyridin-2-yl)-3-methyl-1H-imidazol-3-ium phosphoric acid salt (Compound 26)
(E)-7-chloro-4-(2-(1-(6-(2,4-dimethyl-1H-imidazol-1-yl)pyridin-3-yl)ethylidene)hydrazineyl)quinazoline hydrochloride (Compound 27)
7-chloro-4-(2-(1-(2-(4,5-dimethyl-1H-imidazol-1-yl)pyridin-4-yl)ethylidene)hydrazineyl)quinazoline (Compound 28)
1-(3-(1-(2-(7-chloroquinazolin-4-yl)hydrazineylidene)ethyl)phenyl)—N,N-dimethylmethanamine (Compound 29)
7-chloro-4-(2-(1-(3-(4-methylpiperazin-1-yl)phenyl)ethylidene)hydrazineyl)quinazoline (Compound 30)
6-chloro-N-(6-(4,5-dimethyl-1H-imidazol-1-yl)-5-methoxypyridin-3-yl)phthalazin-1-amine phosphoric acid salt (Compound 31)
(E)-4-(2-(1-(6-(1H-1,2,4-triazol-1-yl)pyridin-3-yl)ethylidene)hydrazineyl)-7-chloroquinazoline (Compound 32)
7-chloro-N-(6-(4,5-dimethyl-11H-imidazol-1-yl)-5-methoxypyridin-3-yl)isoquinolin-4-amine phosphoric acid salt (Compound 33)
N-(6-(1H-1,2,4-triazol-1-yl)pyridin-3-yl)-7-chloroquinazolin-4-amine phosphoric acid salt (Compound 34)
(E)-7-chloro-4-(2-(3-fluoro-4-(1H-imidazol-1-yl)benzylidene)hydrazineyl)quinoline hydrochloride (Compound 35)
7-chloro-N-(6-(2,4-dimethyl-1H-imidazol-1-yl)pyridin-3-yl)isoquinolin-4-amine phosphoric acid salt (Compound 37)
5-((7-chloroquinazolin-4-yl)amino)-2-(4,5-dimethyl-1H-imidazol-1-yl)pyridin-3-ol phosphoric acid salt (Compound 40)
N-(6-(1H-imidazol-1-yl)pyridin-3-yl)-7-chloroquinazolin-4-amine (Compound 41)
7-chloro-N-(3-((diethylamino)methyl)phenyl)quinolin-4-amine (Compound 42)
1-(5-((7-chloroquinazolin-4-yl)amino)pyridin-2-yl)-3-methyl-1H-imidazol-3-ium iodide phosphoric acid salt (Compound 43)
N-(6-(1H-1,2,4-triazol-1-yl)pyridin-3-yl)-7-chloroquinolin-4-amine (Compound 44) and 7-chloro-N-(2-((dimethylamino)methyl)pyridin-4-yl)quinolin-4-amine (Compound 45)

3. A pharmaceutical composition comprising a therapeutic amount of a compound of formula I: 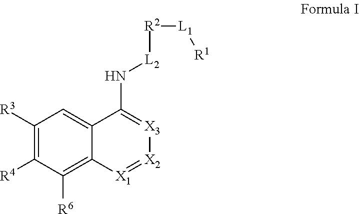
and a pharmaceutically acceptable diluent or carrier,
wherein
X₁, X₂, and X₃ are independent and optionally —CR⁹— or N; R⁹ is alkyl, halogen, —O-alkyl. If X₁ is CR⁹ then R⁹ and R⁶ can be joined to form a 5-7 member carbocyclic, heteroaromatic or heterocyclic ring;
R³ and R⁴ are independent and optionally H, halogen, —O-alkyl. R3 and R4 can be joined to form a fused phenyl;
R⁵ is H, C1-6 alkyl;
R² is phenyl optionally substituted with C1-6 alkyl, —OH, —O-alkyl, halogen, heterocycle, heteroaromatic; 5 or 6 member heterocycle or heteroaromatic optionally substituted with C1-6 alkyl, —OH, —O-alkyl, halogen; a 5 or 6-member heterocycle optionally substituted with alkyl, —O-alkyl, halogen, heterocycle, heteroaromatic; —N+(CH₃)3, optionally substituted 4, 5 and 6-member heterocycle including heterocycles and heteroaromatics that may contain a quaternized nitrogen to form a charged species:
L₁ is a bond, —CH₃, —CH₂—, —[CR7R8]n- where R⁷ and R⁸ are independently: F, C1-6 alkyl, joined to form a C3-6 carbocycle or heterocycle or heteroaromatic.
n=0, 1, 2, 3;
L₂ can be a bond or —N═C(R5)-;
R₁ is null, a 5 or 6-member heterocycle optionally substituted with alkyl, —O-alkyl, halogen; —N+(CH₃)3, optionally substituted 4, 5 and 6-member heterocycle including heterocycles and heteroaromatics that may contain a quaternized nitrogen to form a charged species;
R₆ is alkyl;
wherein the therapeutic amount is effective to inhibit binding of a GAG-binding amyloid peptide (GBAP) to a glycosaminoglycan (GAG).

4. The pharmaceutical composition according to claim 3, wherein the compound of formula I is selected from the group consisting of:
(E)-4-((E)-(4-(1H-imidazol-1-yl)-2-methylbenzylidene)hydrazono)-7-chloro-1,4-dihydroquinoline (Compound 1)
(E)-7-chloro-4-((E)-(4-(4,5-dimethyl-1H-imidazol-1-yl)benzylidene)hydrazono)-1,4-dihydroquinoline (Compound 2)
(E)-4-((E)-(4-(1H-pyrazol-1-yl)benzylidene)hydrazono)-7-chloro-1,4-dihydroquinoline (Compound 3)
(Z)-1-(4-((2-(6-chloroquinolin-4-yl)hydrazineylidene)methyl)phenyl)—N,N,N-trimethylmethanaminium iodide (Compound 4)
(E)-4-(2-(4-(1H-imidazol-1-yl)benzylidene)hydrazinyl)-7-fluoroquinoline (Compound 5)
(E)-4-(4-((2-(7-fluoroquinolin-4-yl)hydrazono)methyl)benzyl)morpholine (Compound 6)
(E)-7-chloro-4-(2-(4-((4-methylpiperazin-1-yl)methyl)benzylidene)hydrazinyl)quinolone
(Compound 7)
(E)-1-(4-((2-(1,2-dihydroacenaphthylen-5-yl)hydrazineylidene)methyl)phenyl)-1H-imidazole (Compound 8)
(Z)-1-methyl-4-((2-(quinolin-4-yl)hydrazono)methyl)pyridin-1-ium iodide (Compound 9)
(E)-4-(4-((2-(7-fluoroquinolin-4-yl)hydrazono)methyl)benzyl)thiomorpholine 1,1-dioxide (Compound 10)
4-(2-(4-(1H-imidazol-1-yl)-3-methylbenzylidene)hydrazineyl)-6,7-dimethoxyquinoline hydrochloride (Compound 11)
4-(2-(4-(1H-imidazol-1-yl)-3-methylbenzylidene)hydrazineyl)-7-methoxyquinoline (Compound 12)
(E)-4-(2-(4-(1H-imidazol-1-yl)-3-methoxybenzylidene)hydrazineyl)-6,7-dimethoxyquinoline phosphate (Compound 13)
(E)-4-(2-((6-(1H-imidazol-1-yl)pyridin-3-yl)methylene)hydrazineyl)-7-chloroquinoline hydrochloride (Compound 14)
7-chloro-4-(2-((6-(4,5-dimethyl-1H-imidazol-1-yl)pyridin-3-yl)methylene)hydrazineyl)quinazoline hydrochloride (Compound 15)
7-chloro-4-(2-(1-(6-(4,5-dimethyl-1H-imidazol-1-yl)pyridin-3-yl)ethylidene)hydrazineyl)quinazoline phosphate (Compound 16)
(E)-1-(5-(1-(2-(7-chloroquinazolin-4-yl)hydrazineylidene)ethyl)pyridin-2-yl)-3-methyl-1H-imidazol-3-ium iodide phosphoric acid salt (Compound 17)
(E)-7-chloro-4-(2-(1-(6-(2,4-dimethyl-1H-imidazol-1-yl)pyridin-3-yl)ethylidene)hydrazineyl)quinazoline phosphoric acid salt (Compound 18)
(E)-4-(2-(1-(6-(2,4-dimethyl-1H-imidazol-1-yl)pyridin-3-yl)ethylidene)hydrazineyl)quinazoline (Compound 19)
7-chloro-N-(2-(4-methylpiperazin-1-yl)pyridin-4-yl)quinolin-4-amine (Compound 20)
(E)-4-(2-(1-(6-(4,5-dimethyl-1H-imidazol-1-yl)pyridin-3-yl)ethylidene)hydrazineyl)-7-fluoroquinazoline (Compound 21)
7-chloro-4-(2-(1-(6-(4,5-dimethyl-1H-imidazol-1-yl)pyridin-3-yl)ethylidene)hydrazineyl)quinoline (Compound 22)
1-(5-(1-(2-(7-chloroquinolin-4-yl)hydrazineylidene)ethyl)pyridin-2-yl)-3-methyl-1H-imidazol-3-ium iodide (Compound 23)
(E)-6-chloro-1-(2-(1-(6-(2,4-dimethyl-1H-imidazol-1-yl)pyridin-3-yl)ethylidene)hydrazineyl)phthalazine (Compound 24)
7-chloro-N-(6-(4,5-dimethyl-1H-imidazol-1-yl)pyridin-3-yl)quinazolin-4-amine (Compound 25)
(E)-1-(5-(1-(2-(7-fluoroquinazolin-4-yl)hydrazineylidene)ethyl)pyridin-2-yl)-3-methyl-1H-imidazol-3-ium phosphoric acid salt (Compound 26)
(E)-7-chloro-4-(2-(1-(6-(2,4-dimethyl-1H-imidazol-1-yl)pyridin-3-yl)ethylidene)hydrazineyl)quinazoline hydrochloride (Compound 27)
7-chloro-4-(2-(1-(2-(4,5-dimethyl-1H-imidazol-1-yl)pyridin-4-yl)ethylidene)hydrazineyl)quinazoline (Compound 28)
1-(3-(1-(2-(7-chloroquinazolin-4-yl)hydrazineylidene)ethyl)phenyl)—N,N-dimethylmethanamine (Compound 29)
7-chloro-4-(2-(1-(3-(4-methylpiperazin-1-yl)phenyl)ethylidene)hydrazineyl)quinazoline (Compound 30)
6-chloro-N-(6-(4,5-dimethyl-1H-imidazol-1-yl)-5-methoxypyridin-3-yl)phthalazin-1-amine phosphoric acid salt (Compound 31)
(E)-4-(2-(1-(6-(1H-1,2,4-triazol-1-yl)pyridin-3-yl)ethylidene)hydrazineyl)-7-chloroquinazoline (Compound 32)
7-chloro-N-(6-(4,5-dimethyl-1H-imidazol-1-yl)-5-methoxypyridin-3-yl)isoquinolin-4-amine phosphoric acid salt (Compound 33)
N-(6-(1H-1,2,4-triazol-1-yl)pyridin-3-yl)-7-chloroquinazolin-4-amine phosphoric acid salt (Compound 34)
(E)-7-chloro-4-(2-(3-fluoro-4-(1H-imidazol-1-yl)benzylidene)hydrazineyl)quinoline hydrochloride (Compound 35)
4-(2-(1-(4-(1H-imidazol-1-yl)phenyl)ethylidene)hydrazineyl)-7-chloroquinoline phosphate (Compound 36)
7-chloro-N-(6-(2,4-dimethyl-1H-imidazol-1-yl)pyridin-3-yl)isoquinolin-4-amine phosphoric acid salt (Compound 37)
4-(2-(4-(1H-imidazol-1-yl)benzylidene)hydrazineyl)-7-chloroquinoline dihydrochloride (Compound 38)
N-(4-(4-ethylpiperazin-1-yl)phenyl)benzo[g]quinolin-4-amine (Compound 39)
5-((7-chloroquinazolin-4-yl)amino)-2-(4,5-dimethyl-1H-imidazol-1-yl)pyridin-3-ol phosphoric acid salt (Compound 40)
N-(6-(1H-imidazol-1-yl)pyridin-3-yl)-7-chloroquinazolin-4-amine (Compound 41) 7-chloro-N-(3-((diethylamino)methyl)phenyl)quinolin-4-amine (Compound 42)
1-(5-((7-chloroquinazolin-4-yl)amino)pyridin-2-yl)-3-methyl-1H-imidazol-3-ium iodide phosphoric acid salt (Compound 43) and
N-(6-(1H-1,2,4-triazol-1-yl)pyridin-3-yl)-7-chloroquinolin-4-amine (Compound 44) and
7-chloro-N-(2-((dimethylamino)methyl)pyridin-4-yl)quinolin-4-amine (Compound 45).

5. The pharmaceutical composition according to claim 3, for the treatment or prevention of neurodegenerative diseases, disorders or conditions.

6. The pharmaceutical composition according to claim 5, wherein the neurodegenerative disease, disorder or condition is Parkinson's Disease, Multiple System Atrophy or an alpha-synucleinopathy.

7. The pharmaceutical composition according to claim 5, wherein the neurodegenerative disease, disorder or condition is Amyotrophic Lateral Sclerosis.

8. The pharmaceutical composition according to claim 5, wherein the neurodegenerative disease, disorder or condition is Alzheimer's Disease or a dementia.

9. The pharmaceutical composition according to claim 3, for the treatment or prevention of an amyloid disease, disorder, or condition.

10. The pharmaceutical composition according to claim 9, wherein the amyloid disease is AA amyloidosis.

11. The pharmaceutical composition according to claim 3, wherein the GBAP is beta-amyloid and the amyloid disease, disorder or condition is Alzheimer's Disease or Cerebral Amyloid Angiopathy.

12. The pharmaceutical composition according to claim 3, wherein the GBAP is tau and the amyloid disease, disorder or condition is Alzheimer's Disease, FTD or a tauopathy; wherein the GBAP is alpha-synuclein and the amyloid neurodegenerative disorder is Parkinson's Disease, Multiple System Atrophy, or an alpha-synucleinopathy; and wherein the GBAP is TDP-43 and the amyloid neurodegenerative disorder is Alzheimer's Disease, Frontotemporal Dementia, or ALS.

13. The pharmaceutical composition according to claim 9, wherein the GBAP is SAA and the amyloid disease, disorder or condition is AA amyloidosis.

14. A method for the treatment or prevention of an amyloid disease, disorder or condition associated with GBAP binding to HS-GAGs comprising:
selecting a subject in need of the treatment or prevention of an amyloid disease, disorder or condition associated with GBAP binding to HS-GAGs;
administering to a subject in need thereof a therapeutic amount of a pharmaceutical composition comprising at least one compound of formula I that inhibits binding of a GAG-binding amyloid peptide (GBAP) to a HS-GAG according to claim 3,
wherein the amyloid disease, disorder or condition associated with GBAP binding to HS-GAGs is treated or prevented in the subject.

15. The method of claim 14, wherein the amyloid disease, disorder or condition is Parkinson's Disease, Multiple System Atrophy, an alpha-synucleinopathy, Alzheimer's Disease, Cerebral Amyloid Angiopathy, tauopathy, Frontotemporal Dementia, ALS, or AA amyloidosis.

16. The method of claim 14, wherein the compound is selected from the group consisting of:
(E)-4-((E)-(4-(1H-imidazol-1-yl)-2-methylbenzylidene)hydrazono)-7-chloro-1,4-dihydroquinoline (Compound 1)
(E)-7-chloro-4-((E)-(4-(4,5-dimethyl-1H-imidazol-1-yl)benzylidene)hydrazono)-1,4-dihydroquinoline (Compound 2)
(E)-4-((E)-(4-(1H-pyrazol-1-yl)benzylidene)hydrazono)-7-chloro-1,4-dihydroquinoline (Compound 3)
(Z)-1-(4-((2-(6-chloroquinolin-4-yl)hydrazineylidene)methyl)phenyl)—N,N,N-trimethylmethanaminium iodide (Compound 4)
(E)-4-(2-(4-(1H-imidazol-1-yl)benzylidene)hydrazinyl)-7-fluoroquinoline (Compound 5)
(E)-4-(4-((2-(7-fluoroquinolin-4-yl)hydrazono)methyl)benzyl)morpholine (Compound 6)
(E)-7-chloro-4-(2-(4-((4-methylpiperazin-1-yl)methyl)benzylidene)hydrazinyl)quinolone (Compound 7)
(E)-1-(4-((2-(1,2-dihydroacenaphthylen-5-yl)hydrazineylidene)methyl)phenyl)-1H-imidazole (Compound 8)
(Z)-1-methyl-4-((2-(quinolin-4-yl)hydrazono)methyl)pyridin-1-ium iodide (Compound 9)
(E)-4-(4-((2-(7-fluoroquinolin-4-yl)hydrazono)methyl)benzyl)thiomorpholine 1,1-dioxide (Compound 10)
4-(2-(4-(1H-imidazol-1-yl)-3-methylbenzylidene)hydrazineyl)-6,7-dimethoxyquinoline hydrochloride (Compound 11)
4-(2-(4-(1H-imidazol-1-yl)-3-methylbenzylidene)hydrazineyl)-7-methoxyquinoline (Compound 12)
(E)-4-(2-(4-(1H-imidazol-1-yl)-3-methoxybenzylidene)hydrazineyl)-6,7-dimethoxyquinoline phosphate (Compound 13)
(E)-4-(2-((6-(1H-imidazol-1-yl)pyridin-3-yl)methylene)hydrazineyl)-7-chloroquinoline hydrochloride (Compound 14)
7-chloro-4-(2-((6-(4,5-dimethyl-1H-imidazol-1-yl)pyridin-3-yl)methylene)hydrazineyl)quinazoline hydrochloride (Compound 15)
7-chloro-4-(2-(1-(6-(4,5-dimethyl-1H-imidazol-1-yl)pyridin-3-yl)ethylidene)hydrazineyl)quinazoline phosphate (Compound 16)
(E)-1-(5-(1-(2-(7-chloroquinazolin-4-yl)hydrazineylidene)ethyl)pyridin-2-yl)-3-methyl-1H-imidazol-3-ium iodide phosphoric acid salt (Compound 17)
(E)-7-chloro-4-(2-(1-(6-(2,4-dimethyl-1H-imidazol-1-yl)pyridin-3-yl)ethylidene)hydrazineyl)quinazoline phosphoric acid salt (Compound 18)
(E)-4-(2-(1-(6-(2,4-dimethyl-1H-imidazol-1-yl)pyridin-3-yl)ethylidene)hydrazineyl)quinazoline (Compound 19)
7-chloro-N-(2-(4-methylpiperazin-1-yl)pyridin-4-yl)quinolin-4-amine (Compound 20)
(E)-4-(2-(1-(6-(4,5-dimethyl-1H-imidazol-1-yl)pyridin-3-yl)ethylidene)hydrazineyl)-7-fluoroquinazoline (Compound 21)
7-chloro-4-(2-(1-(6-(4,5-dimethyl-1H-imidazol-1-yl)pyridin-3-yl)ethylidene)hydrazineyl)quinoline (Compound 22)
1-(5-(1-(2-(7-chloroquinolin-4-yl)hydrazineylidene)ethyl)pyridin-2-yl)-3-methyl-1H-imidazol-3-ium iodide (Compound 23)
(E)-6-chloro-1-(2-(1-(6-(2,4-dimethyl-1H-imidazol-1-yl)pyridin-3-yl)ethylidene)hydrazineyl)phthalazine (Compound 24)
7-chloro-N-(6-(4,5-dimethyl-1H-imidazol-1-yl)pyridin-3-yl)quinazolin-4-amine (Compound 25)
(E)-1-(5-(1-(2-(7-fluoroquinazolin-4-yl)hydrazineylidene)ethyl)pyridin-2-yl)-3-methyl-1H-imidazol-3-ium phosphoric acid salt (Compound 26)
(E)-7-chloro-4-(2-(1-(6-(2,4-dimethyl-1H-imidazol-1-yl)pyridin-3-yl)ethylidene)hydrazineyl)quinazoline hydrochloride (Compound 27)
7-chloro-4-(2-(1-(2-(4,5-dimethyl-1H-imidazol-1-yl)pyridin-4-yl)ethylidene)hydrazineyl)quinazoline (Compound 28)
1-(3-(1-(2-(7-chloroquinazolin-4-yl)hydrazineylidene)ethyl)phenyl)—N,N-dimethylmethanamine (Compound 29)
7-chloro-4-(2-(1-(3-(4-methylpiperazin-1-yl)phenyl)ethylidene)hydrazineyl)quinazoline (Compound 30)
6-chloro-N-(6-(4,5-dimethyl-1H-imidazol-1-yl)-5-methoxypyridin-3-yl)phthalazin-1-amine phosphoric acid salt (Compound 31)
(E)-4-(2-(1-(6-(1H-1,2,4-triazol-1-yl)pyridin-3-yl)ethylidene)hydrazineyl)-7-chloroquinazoline (Compound 32)
7-chloro-N-(6-(4,5-dimethyl-1H-imidazol-1-yl)-5-methoxypyridin-3-yl)isoquinolin-4-amine phosphoric acid salt (Compound 33)
N-(6-(1H-1,2,4-triazol-1-yl)pyridin-3-yl)-7-chloroquinazolin-4-amine phosphoric acid salt (Compound 34)
(E)-7-chloro-4-(2-(3-fluoro-4-(1H-imidazol-1-yl)benzylidene)hydrazineyl)quinoline hydrochloride (Compound 35)
4-(2-(1-(4-(1H-imidazol-1-yl)phenyl)ethylidene)hydrazineyl)-7-chloroquinoline phosphate (Compound 36)
7-chloro-N-(6-(2,4-dimethyl-1H-imidazol-1-yl)pyridin-3-yl)isoquinolin-4-amine phosphoric acid salt (Compound 37)
4-(2-(4-(1H-imidazol-1-yl)benzylidene)hydrazineyl)-7-chloroquinoline dihydrochloride (Compound 38)
N-(4-(4-ethylpiperazin-1-yl)phenyl)benzo[g]quinolin-4-amine (Compound 39)
5-((7-chloroquinazolin-4-yl)amino)-2-(4,5-dimethyl-1H-imidazol-1-yl)pyridin-3-ol phosphoric acid salt (Compound 40)
N-(6-(1H-imidazol-1-yl)pyridin-3-yl)-7-chloroquinazolin-4-amine (Compound 41)
7-chloro-N-(3-((diethylamino)methyl)phenyl)quinolin-4-amine (Compound 42)
1-(5-((7-chloroquinazolin-4-yl)amino)pyridin-2-yl)-3-methyl-1H-imidazol-3-ium iodide phosphoric acid salt (Compound 43) and
N-(6-(1H-1,2,4-triazol-1-yl)pyridin-3-yl)-7-chloroquinolin-4-amine (Compound 44) and
7-chloro-N-(2-((dimethylamino)methyl)pyridin-4-yl)quinolin-4-amine (Compound 45).

17. A method for the treatment or prevention of an amyloid disease, disorder or condition associated with an amyloid peptide binding to HS-GAGs, comprising:
selecting a patient in need of the treatment or prevention of an amyloid disease, disorder or condition associated with an amyloid peptide binding to HS-GAGs;
administering to a subject in need thereof a therapeutic amount of a pharmaceutical composition comprising at least one small molecule compound that inhibits binding of a GAG-binding amyloid peptide (GBAP) to a HS-GAG,
wherein the therapeutic amount is effective to inhibit binding of a GBAP to a HS-GAG, further wherein the amyloid disease, disorder or condition associated with an amyloid peptide binding to HS-GAGs is treated or prevented in the patient.

18. The method according to claim 17, whereas the compound that inhibits binding of a GAG-binding amyloid peptide (GBAP) to a HS-GAG is not a peptide or protein.

19. The method of claim 17, wherein the amyloid disease, disorder or condition is Parkinson's Disease, Multiple System Atrophy, an alpha-synucleinopathy, Alzheimer's Disease, Cerebral Amyloid Angiopathy, tauopathy, Frontotemporal Dementia, ALS, or AA amyloidosis.

20. The method of claim 17, wherein the compound is selected from the group consisting of:
(E)-4-((E)-(4-(1H-imidazol-1-yl)-2-methylbenzylidene)hydrazono)-7-chloro-1,4-dihydroquinoline (Compound 1)
(E)-7-chloro-4-((E)-(4-(4,5-dimethyl-1H-imidazol-1-yl)benzylidene)hydrazono)-1,4-dihydroquinoline (Compound 2)
(E)-4-((E)-(4-(1H-pyrazol-1-yl)benzylidene)hydrazono)-7-chloro-1,4-dihydroquinoline (Compound 3)
(Z)-1-(4-((2-(6-chloroquinolin-4-yl)hydrazineylidene)methyl)phenyl)—N,N,N-trimethylmethanaminium iodide (Compound 4)
(E)-4-(2-(4-(1H-imidazol-1-yl)benzylidene)hydrazinyl)-7-fluoroquinoline (Compound 5)
(E)-4-(4-((2-(7-fluoroquinolin-4-yl)hydrazono)methyl)benzyl)morpholine (Compound 6)
(E)-7-chloro-4-(2-(4-((4-methylpiperazin-1-yl)methyl)benzylidene)hydrazinyl)quinolone (Compound 7)
(E)-1-(4-((2-(1,2-dihydroacenaphthylen-5-yl)hydrazineylidene)methyl)phenyl)-1H-imidazole (Compound 8)
(Z)-1-methyl-4-((2-(quinolin-4-yl)hydrazono)methyl)pyridin-1-ium iodide (Compound 9)
(E)-4-(4-((2-(7-fluoroquinolin-4-yl)hydrazono)methyl)benzyl)thiomorpholine 1,1-dioxide (Compound 10)
4-(2-(4-(1H-imidazol-1-yl)-3-methylbenzylidene)hydrazineyl)-6,7-dimethoxyquinoline hydrochloride (Compound 11)
4-(2-(4-(1H-imidazol-1-yl)-3-methylbenzylidene)hydrazineyl)-7-methoxyquinoline (Compound 12)
(E)-4-(2-(4-(1H-imidazol-1-yl)-3-methoxybenzylidene)hydrazineyl)-6,7-dimethoxyquinoline phosphate (Compound 13)
(E)-4-(2-((6-(1H-imidazol-1-yl)pyridin-3-yl)methylene)hydrazineyl)-7-chloroquinoline hydrochloride (Compound 14)
7-chloro-4-(2-((6-(4,5-dimethyl-1H-imidazol-1-yl)pyridin-3-yl)methylene)hydrazineyl)quinazoline hydrochloride (Compound 15)
7-chloro-4-(2-(1-(6-(4,5-dimethyl-1H-imidazol-1-yl)pyridin-3-yl)ethylidene)hydrazineyl)quinazoline phosphate (Compound 16)
(E)-1-(5-(1-(2-(7-chloroquinazolin-4-yl)hydrazineylidene)ethyl)pyridin-2-yl)-3-methyl-1H-imidazol-3-ium iodide phosphoric acid salt (Compound 17)
(E)-7-chloro-4-(2-(1-(6-(2,4-dimethyl-1H-imidazol-1-yl)pyridin-3-yl)ethylidene)hydrazineyl)quinazoline phosphoric acid salt (Compound 18)
(E)-4-(2-(1-(6-(2,4-dimethyl-1H-imidazol-1-yl)pyridin-3-yl)ethylidene)hydrazineyl)quinazoline (Compound 19)
7-chloro-N-(2-(4-methylpiperazin-1-yl)pyridin-4-yl)quinolin-4-amine (Compound 20)
(E)-4-(2-(1-(6-(4,5-dimethyl-1H-imidazol-1-yl)pyridin-3-yl)ethylidene)hydrazineyl)-7-fluoroquinazoline (Compound 21)
7-chloro-4-(2-(1-(6-(4,5-dimethyl-1H-imidazol-1-yl)pyridin-3-yl)ethylidene)hydrazineyl)quinoline (Compound 22)
1-(5-(1-(2-(7-chloroquinolin-4-yl)hydrazineylidene)ethyl)pyridin-2-yl)-3-methyl-1H-imidazol-3-ium iodide (Compound 23)
(E)-6-chloro-1-(2-(1-(6-(2,4-dimethyl-1H-imidazol-1-yl)pyridin-3-yl)ethylidene)hydrazineyl)phthalazine (Compound 24)
7-chloro-N-(6-(4,5-dimethyl-1H-imidazol-1-yl)pyridin-3-yl)quinazolin-4-amine (Compound 25)
(E)-1-(5-(1-(2-(7-fluoroquinazolin-4-yl)hydrazineylidene)ethyl)pyridin-2-yl)-3-methyl-1H-imidazol-3-ium phosphoric acid salt (Compound 26)
(E)-7-chloro-4-(2-(1-(6-(2,4-dimethyl-1H-imidazol-1-yl)pyridin-3-yl)ethylidene)hydrazineyl)quinazoline hydrochloride (Compound 27)
7-chloro-4-(2-(1-(2-(4,5-dimethyl-1H-imidazol-1-yl)pyridin-4-yl)ethylidene)hydrazineyl)quinazoline (Compound 28)
1-(3-(1-(2-(7-chloroquinazolin-4-yl)hydrazineylidene)ethyl)phenyl)—N,N-dimethylmethanamine (Compound 29)
7-chloro-4-(2-(1-(3-(4-methylpiperazin-1-yl)phenyl)ethylidene)hydrazineyl)quinazoline (Compound 30)
6-chloro-N-(6-(4,5-dimethyl-1H-imidazol-1-yl)-5-methoxypyridin-3-yl)phthalazin-1-amine phosphoric acid salt (Compound 31)
(E)-4-(2-(1-(6-(1H-1,2,4-triazol-1-yl)pyridin-3-yl)ethylidene)hydrazineyl)-7-chloroquinazoline (Compound 32)
7-chloro-N-(6-(4,5-dimethyl-1H-imidazol-1-yl)-5-methoxypyridin-3-yl)isoquinolin-4-amine phosphoric acid salt (Compound 33)
N-(6-(1H-1,2,4-triazol-1-yl)pyridin-3-yl)-7-chloroquinazolin-4-amine phosphoric acid salt (Compound 34)
(E)-7-chloro-4-(2-(3-fluoro-4-(1H-imidazol-1-yl)benzylidene)hydrazineyl)quinoline hydrochloride (Compound 35)
4-(2-(1-(4-(1H-imidazol-1-yl)phenyl)ethylidene)hydrazineyl)-7-chloroquinoline phosphate (Compound 36)
7-chloro-N-(6-(2,4-dimethyl-1H-imidazol-1-yl)pyridin-3-yl)isoquinolin-4-amine phosphoric acid salt (Compound 37)
4-(2-(4-(1H-imidazol-1-yl)benzylidene)hydrazineyl)-7-chloroquinoline dihydrochloride (Compound 38)
N-(4-(4-ethylpiperazin-1-yl)phenyl)benzo[g]quinolin-4-amine (Compound 39)
5-((7-chloroquinazolin-4-yl)amino)-2-(4,5-dimethyl-1H-imidazol-1-yl)pyridin-3-ol phosphoric acid salt (Compound 40)
N-(6-(1H-imidazol-1-yl)pyridin-3-yl)-7-chloroquinazolin-4-amine (Compound 41)
7-chloro-N-(3-((diethylamino)methyl)phenyl)quinolin-4-amine (Compound 42)
1-(5-((7-chloroquinazolin-4-yl)amino)pyridin-2-yl)-3-methyl-1H-imidazol-3-ium iodide phosphoric acid salt (Compound 43) and
N-(6-(1H-1,2,4-triazol-1-yl)pyridin-3-yl)-7-chloroquinolin-4-amine (Compound 44) and
7-chloro-N-(2-((dimethylamino)methyl)pyridin-4-yl)quinolin-4-amine (Compound 45).

21. A method for detecting a small molecule compound that directly binds to a glycosaminoglycan (GAG) comprising:
(a) immobilizing a GAG to a surface of a multi-well plate;
(b) contacting immobilized GAG with a known quantity of GBAP in the presence of at least one candidate compound; and
(c) measuring the amount of the GBAP bound to the immobilized GAG,
wherein the small organic molecule compound is effective to inhibit binding of the GBAP to the GAG.

22. A method for detecting a small organic molecule that directly binds to a glycosaminoglycan (GAG) comprising:
(a) immobilizing a GAG to a surface of a multi-well plate
(b) contacting a GAG with at least one candidate small organic compound;
(c) removing unbound small organic compound;
(d) adding a GBAP; and
(e) measuring the amount of the GBAP bound to the immobilized GAG,
wherein the small molecule compound is effective to inhibit binding of the GBAP to the GAG.

23. The method according to claim 21, wherein the GBAP is selected from the group consisting of amyloid-beta, alpha-synuclein, TDP-43, tau, SAA, IAPP, and derivatives and fragments thereof.

24. A method for treatment or prevention of an amyloid neurodegenerative disorder comprising:
selecting a subject in need of the treatment or prevention of an amyloid neurodegenerative disorder;
administering to the subject a therapeutic amount of a compound that is a dual inhibitor of GBAP binding to a HS-GAG, wherein the two GBAPs are selected from the group consisting of Abeta, tau, TDP-43 and alpha-synuclein,
wherein the amyloid neurodegenerative disorder is treated or prevented in the subject.

25. The method of treatment according to claim 24 whereas the two GBAPs are Abeta and tau, wherein the amyloid neurodegenerative disorder is Alzheimer's Disease.

26. The method according to claim 24, wherein the GBAPs are tau and TDP-43 and the amyloid disorder is FTD.

27. The method according to claim 24 wherein the amyloid neurodegenerative disorder is Parkinson's Disease, Multiple System Atrophy, an alpha-synucleinopathy, Alzheimer's Disease, Cerebral Amyloid Angiopathy, tauopathy, Frontotemporal Dementia, ALS, or AA amyloidosis.

28. The method of claim 24, wherein the compound is selected from the group consisting of:
(E)-4-((E)-(4-(1H-imidazol-1-yl)-2-methylbenzylidene)hydrazono)-7-chloro-1,4-dihydroquinoline (Compound 1)
(E)-7-chloro-4-((E)-(4-(4,5-dimethyl-11H-imidazol-1-yl)benzylidene)hydrazono)-1,4-dihydroquinoline (Compound 2)
(E)-4-((E)-(4-(1H-pyrazol-1-yl)benzylidene)hydrazono)-7-chloro-1,4-dihydroquinoline (Compound 3)
(Z)-1-(4-((2-(6-chloroquinolin-4-yl)hydrazineylidene)methyl)phenyl)—N,N,N-trimethylmethanaminium iodide (Compound 4)
(E)-4-(2-(4-(1H-imidazol-1-yl)benzylidene)hydrazinyl)-7-fluoroquinoline (Compound 5)
(E)-4-(4-((2-(7-fluoroquinolin-4-yl)hydrazono)methyl)benzyl)morpholine (Compound 6)
(E)-7-chloro-4-(2-(4-((4-methylpiperazin-1-yl)methyl)benzylidene)hydrazinyl)quinolone (Compound 7)
(E)-1-(4-((2-(1,2-dihydroacenaphthylen-5-yl)hydrazineylidene)methyl)phenyl)-1H-imidazole (Compound 8)
(Z)-1-methyl-4-((2-(quinolin-4-yl)hydrazono)methyl)pyridin-1-ium iodide (Compound 9)
(E)-4-(4-((2-(7-fluoroquinolin-4-yl)hydrazono)methyl)benzyl)thiomorpholine 1,1-dioxide (Compound 10)
4-(2-(4-(1H-imidazol-1-yl)-3-methylbenzylidene)hydrazineyl)-6,7-dimethoxyquinoline hydrochloride (Compound 11)
4-(2-(4-(1H-imidazol-1-yl)-3-methylbenzylidene)hydrazineyl)-7-methoxyquinoline (Compound 12)
(E)-4-(2-(4-(1H-imidazol-1-yl)-3-methoxybenzylidene)hydrazineyl)-6,7-dimethoxyquinoline phosphate (Compound 13)
(E)-4-(2-((6-(1H-imidazol-1-yl)pyridin-3-yl)methylene)hydrazineyl)-7-chloroquinoline hydrochloride (Compound 14)
7-chloro-4-(2-((6-(4,5-dimethyl-1H-imidazol-1-yl)pyridin-3-yl)methylene)hydrazineyl)quinazoline hydrochloride (Compound 15)
7-chloro-4-(2-(1-(6-(4,5-dimethyl-1H-imidazol-1-yl)pyridin-3-yl)ethylidene)hydrazineyl)quinazoline phosphate (Compound 16)
(E)-1-(5-(1-(2-(7-chloroquinazolin-4-yl)hydrazineylidene)ethyl)pyridin-2-yl)-3-methyl-1H-imidazol-3-ium iodide phosphoric acid salt (Compound 17)
(E)-7-chloro-4-(2-(1-(6-(2,4-dimethyl-1H-imidazol-1-yl)pyridin-3-yl)ethylidene)hydrazineyl)quinazoline phosphoric acid salt (Compound 18)
(E)-4-(2-(1-(6-(2,4-dimethyl-1H-imidazol-1-yl)pyridin-3-yl)ethylidene)hydrazineyl)quinazoline (Compound 19) 7-chloro-N-(2-(4-methylpiperazin-1-yl)pyridin-4-yl)quinolin-4-amine (Compound 20)
(E)-4-(2-(1-(6-(4,5-dimethyl-1H-imidazol-1-yl)pyridin-3-yl)ethylidene)hydrazineyl)-7-fluoroquinazoline (Compound 21)
7-chloro-4-(2-(1-(6-(4,5-dimethyl-1H-imidazol-1-yl)pyridin-3-yl)ethylidene)hydrazineyl)quinoline (Compound 22)
1-(5-(1-(2-(7-chloroquinolin-4-yl)hydrazineylidene)ethyl)pyridin-2-yl)-3-methyl-1H-imidazol-3-ium iodide (Compound 23)
(E)-6-chloro-1-(2-(1-(6-(2,4-dimethyl-1H-imidazol-1-yl)pyridin-3-yl)ethylidene)hydrazineyl)phthalazine (Compound 24)
7-chloro-N-(6-(4,5-dimethyl-1H-imidazol-1-yl)pyridin-3-yl)quinazolin-4-amine (Compound 25)
(E)-1-(5-(1-(2-(7-fluoroquinazolin-4-yl)hydrazineylidene)ethyl)pyridin-2-yl)-3-methyl-1H-imidazol-3-ium phosphoric acid salt (Compound 26)
(E)-7-chloro-4-(2-(1-(6-(2,4-dimethyl-1H-imidazol-1-yl)pyridin-3-yl)ethylidene)hydrazineyl)quinazoline hydrochloride (Compound 27)
7-chloro-4-(2-(1-(2-(4,5-dimethyl-1H-imidazol-1-yl)pyridin-4-yl)ethylidene)hydrazineyl)quinazoline (Compound 28)
1-(3-(1-(2-(7-chloroquinazolin-4-yl)hydrazineylidene)ethyl)phenyl)—N,N-dimethylmethanamine (Compound 29)
7-chloro-4-(2-(1-(3-(4-methylpiperazin-1-yl)phenyl)ethylidene)hydrazineyl)quinazoline (Compound 30)
6-chloro-N-(6-(4,5-dimethyl-1H-imidazol-1-yl)-5-methoxypyridin-3-yl)phthalazin-1-amine phosphoric acid salt (Compound 31)
(E)-4-(2-(1-(6-(1H-1,2,4-triazol-1-yl)pyridin-3-yl)ethylidene)hydrazineyl)-7-chloroquinazoline (Compound 32)
7-chloro-N-(6-(4,5-dimethyl-1H-imidazol-1-yl)-5-methoxypyridin-3-yl)isoquinolin-4-amine phosphoric acid salt (Compound 33)
N-(6-(1H-1,2,4-triazol-1-yl)pyridin-3-yl)-7-chloroquinazolin-4-amine phosphoric acid salt (Compound 34)
(E)-7-chloro-4-(2-(3-fluoro-4-(1H-imidazol-1-yl)benzylidene)hydrazineyl)quinoline hydrochloride (Compound 35)
4-(2-(1-(4-(1H-imidazol-1-yl)phenyl)ethylidene)hydrazineyl)-7-chloroquinoline phosphate (Compound 36)
7-chloro-N-(6-(2,4-dimethyl-1H-imidazol-1-yl)pyridin-3-yl)isoquinolin-4-amine phosphoric acid salt (Compound 37)
4-(2-(4-(1H-imidazol-1-yl)benzylidene)hydrazineyl)-7-chloroquinoline dihydrochloride (Compound 38)
N-(4-(4-ethylpiperazin-1-yl)phenyl)benzo[g]quinolin-4-amine (Compound 39) 5-((7-chloroquinazolin-4-yl)amino)-2-(4,5-dimethyl-1H-imidazol-1-yl)pyridin-3-ol phosphoric acid salt (Compound 40)
N-(6-(1H-imidazol-1-yl)pyridin-3-yl)-7-chloroquinazolin-4-amine (Compound 41) 7-chloro-N-(3-((diethylamino)methyl)phenyl)quinolin-4-amine (Compound 42)
1-(5-((7-chloroquinazolin-4-yl)amino)pyridin-2-yl)-3-methyl-1H-imidazol-3-ium iodide phosphoric acid salt (Compound 43) and
N-(6-(1H-1,2,4-triazol-1-yl)pyridin-3-yl)-7-chloroquinolin-4-amine (Compound 44) and
7-chloro-N-(2-((dimethylamino)methyl)pyridin-4-yl)quinolin-4-amine (Compound 45).

29. A method of treating an amyloid disorder comprising:
selecting a subject in need of treatment of an amyloid disorder;
administering to a subject in need thereof a therapeutic amount of a compound that binds to a glycosaminoglycan (GAG), wherein the therapeutic amount is effective to inhibit the GAG-binding amyloid peptide (GBAP) binding to the GAG,
wherein the amyloid disorder is treated in the subject.

30. A method according to claim 29 wherein the GAG is a heparan sulfate GAG (HS-GAG).

31. The method according to claim 29 wherein the amyloid neurodegenerative disorder is Parkinson's Disease, Multiple System Atrophy, an alpha-synucleinopathy, Alzheimer's Disease, Cerebral Amyloid Angiopathy, tauopathy, Frontotemporal Dementia, ALS, or AA amyloidosis.

32. The method of claim 29, wherein the compound is selected from the group consisting of:
(E)-4-((E)-(4-(1H-imidazol-1-yl)-2-methylbenzylidene)hydrazono)-7-chloro-1,4-dihydroquinoline (Compound 1)
(E)-7-chloro-4-((E)-(4-(4,5-dimethyl-1H-imidazol-1-yl)benzylidene)hydrazono)-1,4-dihydroquinoline (Compound 2)
(E)-4-((E)-(4-(1H-pyrazol-1-yl)benzylidene)hydrazono)-7-chloro-1,4-dihydroquinoline (Compound 3)
(Z)-1-(4-((2-(6-chloroquinolin-4-yl)hydrazineylidene)methyl)phenyl)—N,N,N-trimethylmethanaminium iodide (Compound 4)
(E)-4-(2-(4-(1H-imidazol-1-yl)benzylidene)hydrazinyl)-7-fluoroquinoline (Compound 5)
(E)-4-(4-((2-(7-fluoroquinolin-4-yl)hydrazono)methyl)benzyl)morpholine (Compound 6)
(E)-7-chloro-4-(2-(4-((4-methylpiperazin-1-yl)methyl)benzylidene)hydrazinyl)quinolone (Compound 7)
(E)-1-(4-((2-(1,2-dihydroacenaphthylen-5-yl)hydrazineylidene)methyl)phenyl)-1H-imidazole (Compound 8)
(Z)-1-methyl-4-((2-(quinolin-4-yl)hydrazono)methyl)pyridin-1-ium iodide (Compound 9)
(E)-4-(4-((2-(7-fluoroquinolin-4-yl)hydrazono)methyl)benzyl)thiomorpholine 1,1-dioxide (Compound 10)
4-(2-(4-(1H-imidazol-1-yl)-3-methylbenzylidene)hydrazineyl)-6,7-dimethoxyquinoline hydrochloride (Compound 11)
4-(2-(4-(1H-imidazol-1-yl)-3-methylbenzylidene)hydrazineyl)-7-methoxyquinoline (Compound 12)
(E)-4-(2-(4-(1H-imidazol-1-yl)-3-methoxybenzylidene)hydrazineyl)-6,7-dimethoxyquinoline phosphate (Compound 13)
(E)-4-(2-((6-(1H-imidazol-1-yl)pyridin-3-yl)methylene)hydrazineyl)-7-chloroquinoline hydrochloride (Compound 14)
7-chloro-4-(2-((6-(4,5-dimethyl-1H-imidazol-1-yl)pyridin-3-yl)methylene)hydrazineyl)quinazoline hydrochloride (Compound 15)
7-chloro-4-(2-(1-(6-(4,5-dimethyl-1H-imidazol-1-yl)pyridin-3-yl)ethylidene)hydrazineyl)quinazoline phosphate (Compound 16)
(E)-1-(5-(1-(2-(7-chloroquinazolin-4-yl)hydrazineylidene)ethyl)pyridin-2-yl)-3-methyl-1H-imidazol-3-ium iodide phosphoric acid salt (Compound 17)
(E)-7-chloro-4-(2-(1-(6-(2,4-dimethyl-1H-imidazol-1-yl)pyridin-3-yl)ethylidene)hydrazineyl)quinazoline phosphoric acid salt (Compound 18)
(E)-4-(2-(1-(6-(2,4-dimethyl-1H-imidazol-1-yl)pyridin-3-yl)ethylidene)hydrazineyl)quinazoline (Compound 19)
7-chloro-N-(2-(4-methylpiperazin-1-yl)pyridin-4-yl)quinolin-4-amine (Compound 20)
(E)-4-(2-(1-(6-(4,5-dimethyl-1H-imidazol-1-yl)pyridin-3-yl)ethylidene)hydrazineyl)-7-fluoroquinazoline (Compound 21)
7-chloro-4-(2-(1-(6-(4,5-dimethyl-1H-imidazol-1-yl)pyridin-3-yl)ethylidene)hydrazineyl)quinoline (Compound 22)
1-(5-(1-(2-(7-chloroquinolin-4-yl)hydrazineylidene)ethyl)pyridin-2-yl)-3-methyl-1H-imidazol-3-ium iodide (Compound 23)
(E)-6-chloro-1-(2-(1-(6-(2,4-dimethyl-1H-imidazol-1-yl)pyridin-3-yl)ethylidene)hydrazineyl)phthalazine (Compound 24)
7-chloro-N-(6-(4,5-dimethyl-1H-imidazol-1-yl)pyridin-3-yl)quinazolin-4-amine (Compound 25)
(E)-1-(5-(1-(2-(7-fluoroquinazolin-4-yl)hydrazineylidene)ethyl)pyridin-2-yl)-3-methyl-1H-imidazol-3-ium phosphoric acid salt (Compound 26)
(E)-7-chloro-4-(2-(1-(6-(2,4-dimethyl-1H-imidazol-1-yl)pyridin-3-yl)ethylidene)hydrazineyl)quinazoline hydrochloride (Compound 27)
7-chloro-4-(2-(1-(2-(4,5-dimethyl-1H-imidazol-1-yl)pyridin-4-yl)ethylidene)hydrazineyl)quinazoline (Compound 28)
1-(3-(1-(2-(7-chloroquinazolin-4-yl)hydrazineylidene)ethyl)phenyl)—N,N-dimethylmethanamine (Compound 29)
7-chloro-4-(2-(1-(3-(4-methylpiperazin-1-yl)phenyl)ethylidene)hydrazineyl)quinazoline (Compound 30)
6-chloro-N-(6-(4,5-dimethyl-1H-imidazol-1-yl)-5-methoxypyridin-3-yl)phthalazin-1-amine phosphoric acid salt (Compound 31)
(E)-4-(2-(1-(6-(1H-1,2,4-triazol-1-yl)pyridin-3-yl)ethylidene)hydrazineyl)-7-chloroquinazoline (Compound 32)
7-chloro-N-(6-(4,5-dimethyl-1H-imidazol-1-yl)-5-methoxypyridin-3-yl)isoquinolin-4-amine phosphoric acid salt (Compound 33)
N-(6-(1H-1,2,4-triazol-1-yl)pyridin-3-yl)-7-chloroquinazolin-4-amine phosphoric acid salt (Compound 34)
(E)-7-chloro-4-(2-(3-fluoro-4-(1H-imidazol-1-yl)benzylidene)hydrazineyl)quinoline hydrochloride (Compound 35)
4-(2-(1-(4-(11H-imidazol-1-yl)phenyl)ethylidene)hydrazineyl)-7-chloroquinoline phosphate (Compound 36)
7-chloro-N-(6-(2,4-dimethyl-1H-imidazol-1-yl)pyridin-3-yl)isoquinolin-4-amine phosphoric acid salt (Compound 37)
4-(2-(4-(1H-imidazol-1-yl)benzylidene)hydrazineyl)-7-chloroquinoline dihydrochloride (Compound 38)
N-(4-(4-ethylpiperazin-1-yl)phenyl)benzo[g]quinolin-4-amine (Compound 39)
5-((7-chloroquinazolin-4-yl)amino)-2-(4,5-dimethyl-1H-imidazol-1-yl)pyridin-3-ol phosphoric acid salt (Compound 40)
N-(6-(1H-imidazol-1-yl)pyridin-3-yl)-7-chloroquinazolin-4-amine (Compound 41) 7-chloro-N-(3-((diethylamino)methyl)phenyl)quinolin-4-amine (Compound 42)
1-(5-((7-chloroquinazolin-4-yl)amino)pyridin-2-yl)-3-methyl-1H-imidazol-3-ium iodide phosphoric acid salt (Compound 43) and
N-(6-(1H-1,2,4-triazol-1-yl)pyridin-3-yl)-7-chloroquinolin-4-amine (Compound 44) and
7-chloro-N-(2-((dimethylamino)methyl)pyridin-4-yl)quinolin-4-amine (Compound 45).
